(19) United States
(12) Patent Application Publication
(10) Pub. No.: US 2015/0361500 A1
(43) Pub. Date: Dec. 17, 2015

(54) BIOMARKERS FOR DIAGNOSIS AND TREATMENT OF ACNE VULGARIS

(71) Applicant: DermTech International, La Jolla, CA (US)

(72) Inventors: Sherman H. Chang, San Diego, CA (US); Tara J. Palmer, San Diego, CA (US)

(21) Appl. No.: 14/832,964

(22) Filed: Aug. 21, 2015

Related U.S. Application Data
(63) Continuation of application No. 14/261,361, filed on Apr. 24, 2014.
(60) Provisional application No. 61/816,618, filed on Apr. 26, 2013.

(51) Int. Cl. C12Q 1/68

(52) U.S. Cl. CPC C12Q 1/6883; C12Q 2600/16; C12Q 2600/158; C12Q 2600/118

(57) ABSTRACT

Described herein are methods, systems, platforms, and kits for the characterization, assessment, diagnosis and treatment of acne vulgaris. Among the methods provided are methods of identifying a gene expression profile for acne vulgaris and biomarkers for monitoring treatment. Also provided are methods, systems, platforms, and kits for monitoring efficacy of a treatment and methods for selecting a treatment regimen.

### CROSS-REFERENCE TO RELATED APPLICATIONS

This application is a continuation of co-pending U.S. application Ser. No. 14/261,361 filed on Apr. 24, 2014, which claims the benefit of U.S. Provisional Application No. 61/816,618, filed Apr. 26, 2013, which is incorporated by reference herein in its entirety.

### SUMMARY OF THE INVENTION

Described herein, in certain embodiments, are methods for the diagnosis, characterization, assessment, and/or treatment of acne vulgaris.

Described herein, in certain embodiments, are methods for determining the response of a subject to a treatment for acne vulgaris comprising: applying an adhesive tape to an acne lesion of the subject in a manner sufficient to isolate an epidermal sample adhering to the adhesive tape, wherein the epidermal sample, comprising a gene product expressed by one or more genes, or the gene itself, that is listed in Tables 3, 6, 7, 8, or 9, and optionally that is obtained at a time point following administration of a treatment for acne vulgaris; and characterizing the subject as being responsive to the treatment based on the relative amount of the gene product present in the epidermal sample. In some embodiments, the gene is a gene listed in Table 3 or the gene product is expressed by a gene selected from Table 3. In some embodiments, the gene is one that is listed in Table 6 or is a gene product that is expressed by a gene selected from Table 6. In some embodiments, the gene or gene expression product is of a gene selected from or listed within Table 7. In some embodiments, the gene product is expressed by a gene selected from Table 8 or the gene is a gene from Table 8. In some embodiments, the gene is a gene listed on Table 9 or the gene product is expressed by a gene selected from Table 9. In some embodiments, the gene or gene product is one that has increased expression in inflammatory acne lesions compared to normal skin. In some embodiments, the gene or gene product is one that has decreased expression in inflammatory acne lesions compared to normal skin. In some embodiments, the or gene or expression product comprises defensin beta 4 (DEFB4), 5100 calcium binding protein A8 (calgranulin B) (S100A8), Transcribed locus, SH3-domain GRB2-like 3, cysteinyl leukotriene receptor 1 (CYSLTR1), hypothetical protein FLJ10808, programmed cell death 7 (PDCD7), Wilms tumor 1 associated protein (WTAP), aprataxin (APTX), serpin peptidase inhibitor clade B (ovalbumin) member 3 (SERPINB3), solute carrier family 16 member 10 (SLC16A10), interleukin 8 (IL-8), cystatin E/M (CST6), serine peptidase inhibitor Kazal type 5 (SPINK5), TIMP metallopeptidase inhibitor 3 (TIMP3), ICEBERG caspase-1 inhibitor, serpin peptidase inhibitor, clade A (alpha-1 antiproteinase, andtitrypsin) member 3 (SERPINA3), WAP four-disulfide core domain 3 (WFCD3), cysteine rich transmembrane BMP regulator 1 (chordin-like) (CRIM1), or serine peptidase inhibitor Kunitz type 2 (SPINT2), or combinations thereof. In some embodiments, the gene or gene expression product comprises S100 calcium binding protein A8 (calgranulin B) (S100A8), Transcribed locus (GenBank Accession AI741601), SH3-domain GRB2-like 3 (GenBank Accession AK098337), cysteinyl leukotriene receptor 1 (CYSLTR1), ubiquitin-like modifier activating enzyme 6 (UBA6), programmed cell death 7 (PDCD7), Wilms tumor 1 associated protein (WTAP), aprataxin (APTX), serpin peptidase inhibitor clade B (ovalbumin) member 3 (SERPINB3), solute carrier family 16 member 10 (SLC16A10), cystatin E/M (CST6), serine peptidase inhibitor Kazal type 5 (SPINK5), TIMP metallopeptidase inhibitor 3 (TIMP3), caspase recruitment domain family, member 18 (CARD18), serpin peptidase inhibitor, Glade A (alpha-1 antiproteinase, andtitrypsin) member 3 (SERPINA3), WAP four-disulfide core domain 3 (WFCD3), cysteine rich transmembrane BMP regulator 1 (chordin-like) (CRIM1), or serine peptidase inhibitor Kunitz type 2 (SPINT2), or combinations thereof. In some embodiments, the gene or expression product is isolated, purified, or both. In some embodiments, the method futher comprises detecting the relative amount of the gene or the one or more gene products compared to a control. In some embodiments, the gene product is a nucleic acid molecule or a protein. In some embodiments, the nucleic acid molecule is an RNA molecule. In some embodiments, the control is the relative amount of the gene that is present or its expression product that is expressed in an epidermal skin sample obtained from the subject prior to treatment. In some embodiments, the control is the relative amount of the gene that is present or the gene product expressed in an epidermal skin sample obtained from an inflammatory acne lesion. In some embodiments, the control is the relative amount of the gene product expressed in an epidermal skin sample obtained from an inflammatory acne lesion of the subject prior to treatment. In some embodiments, the relative amount of the gene or gene product is decreased compared to the control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the relative amount of the gene or gene product is increased compared to the control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the relative amount of two or more genes, or gene products expressed by one or more genes listed, in Tables 3, 6, 7, 8, or 9 are detected. In some embodiments, the methods further comprise detecting the relative amount of two or more genes, or gene products expressed by one or more genes listed, of any of Tables 3, 6, 7, 8, or 9. In some embodiments, the methods further comprise detecting the relative amount of two, 3 or more, 4 or more, 5 or more, 6 or more, 7 or more, 8 or more, 9 or more, or 10 or more genes listed in any of Tables 3, 6, 7, 8, or 9 or gene products expressed by one or more genes listed in any of Tables 3, 6, 7, 8, or 9. In some embodiments, the methods further comprise detecting the relative amount of two, 3, 4, 5, 6, 7, 8, 9, or 10 genes, or gene products expressed by one or more genes, listed in any of Tables 3, 6, 7, 8, or 9. In some embodiments, the methods further comprise detecting the relative amount of 2, 3, 4, 5, 6, 7, 8, 9, 10 genes, or gene products expressed by one or more genes listed in Table 3. In some embodiments, the methods further comprise detecting the relative amount of 2, 3, 4, 5, 6, 7, 8, 9, 10 genes, or gene products expressed by one or more genes, listed in Table 6. In some embodiments, the methods further comprise detecting the relative amount of 2, 3, 4, 5, 6, 7, 8, 9, 10 genes, or gene products expressed by one or more genes, listed in Table 7. In some embodiments, the methods further comprise detecting the relative amount of 2, 3, 4, 5, 6, 7, 8, 9, 10 or more genes, or gene products expressed by one or more genes, listed in Table 8. In some embodiments, the methods further comprise detecting the relative amount of 2, 3, 4, 5, 6, 7, 8, 9, 10 or more genes, or gene products expressed by one or more genes, listed in Table 9. In some embodiments, the methods further comprise detecting the relative amount of a gene or a gene product expressed by one or more genes listed in Table 9 and a gene or gene product expressed by one or more genes listed in any of Tables 3, 6, 7, 8. In some embodiments, the methods further comprise detecting the relative amount of a gene that is, or an expression product expressed by, DEFB4 and a gene listed or a gene product expressed by one or more genes listed in Table 9. In some embodiments, the methods further comprise detecting the relative amount of a gene that is, or a gene product expressed by, TIMP3 and a gene product expressed by one or more genes listed in Table 9. In some embodiments, the methods further comprise detecting the relative amount of a gene that comprises, or a gene product expressed by, IL8 and a gene that comprises, or its expression product expressed by, one or more genes listed in Table 9. In some embodiments, the gene or expression product is isolated, purified, or both. In some embodiments, the methods further comprise isolating the nucleic acid or in certain cases protein, from the epidermal sample. In some embodiments, the methods further comprise applying the isolated nucleic acid molecule to a microarray. In some embodiments, the methods further comprise amplifying the nucleic acid molecule from the sample prior to detecting. In some embodiments, the methods further comprise applying the amplification product thereof to a microarray. In some embodiments, detecting comprises measuring the amount of the nucleic acid hybridized to the microarray. In some embodiments, detecting comprises quantitative polymerase chain reaction. In some embodiments, 1, 2, 3, 4, 5, 6, 7, 8, 9, or 10 adhesive tapes are applied and removed from the skin In some embodiments, 1, 2, 3, 4, 5, 6, 7, 8, 9, or 10 applications of a tape are applied and removed from the skin. In some embodiments, 1, 2, 3, 4, 5, 6, 7, 8, 9, or 10 adhesive tapes are applied to the same site sequentially. In some embodiments, 1, 2, 3, 4, 5, 6, 7, 8, 9, or 10 adhesive tapes are applied to different sites. In some embodiments, the adhesive tape comprises a rubber adhesive on a polyurethane film. In some embodiments, the methods comprise applying the adhesive tape to the skin of the face, upper back, or upper chest of the subject. In some embodiments, the methods further comprise continuing the treatment if the subject is characterized as being responsive to the treatment. In some embodiments, the methods further comprise ceasing the treatment if the subject is not characterized as being responsive to the treatment. In some embodiments, the methods further comprise increasing the frequency of the treatment. In some embodiments, the methods further comprise increasing the dosage of the treatment. In some embodiments, the methods further comprise administering an additional treatment for acne vulgaris. In some embodiments, the methods further comprise obtaining the epidermal sample at 1 days, 2 days, 3 days, 4 days, 5 days, 6 days, 1 week, 2 weeks, 3 weeks, 4 weeks, 5 weeks, 6 weeks, 7 weeks, 8 weeks, 9 weeks, or 10 weeks following treatment of the subject for acne vulgaris. In some embodiments, the methods further comprise administering the treatment orally or topically to the skin. In some embodiments, the treatment comprises an antibiotic, a retinoid, a hormone, or an aldosterone receptor antagonist. In some embodiments, the treatment comprises benzoyl peroxide, asapalene, azalaic acid, clindamycin, cephalexin, dapsone, dropirenone, doxycycline, erythromycin. ethinyl estradiol, isotretinoin, magnesium hydroxide, minocycline, salicylic acid, sodium sulfacetamide, sulfamethoxazole, spironolactone, tazarotene, tretinoin or trimethoprim. In some embodiments, the methods further comprise obtaining multiple epidermal skin samples at successive time points over the course of treatment. In some embodiments, the methods further comprise monitoring the expression of the one or more gene products at successive time points over the course of treatment.

Described herein in certain embodiments are methods for characterizing tissue comprising: applying an adhesive tape to tissue of a subject in a manner sufficient to isolate an epidermal sample adhering to the adhesive tape, wherein the epidermal sample comprises a gene that is, or a gene product expressed by one or more genes, listed in Tables 3, 6, 7, 8, or 9; and characterizing the subject as having acne vulgaris based on the relative amount of the gene or gene product present in the epidermal sample. In some embodiments, the gene is identified on, or the expression product is expressed by a gene that comprises one listed on, Table 3. In some embodiments, the gene comprises, or the expression product is expressed by a gene that comprises one listed on, Table 6. In some embodiments, the gene comprises a gene identified on Table 7 or an expression product of a gene from Table 7. In some embodiments, the gene or gene product is expressed by a gene selected from Table 8. In some embodiments, the gene or its expression product is a gene from Table 9. In some embodiments, the gene or expression product is one that has increased expression in inflammatory acne lesions compared to normal skin. In some embodiments, the gene or expression product is one that has decreased expression in inflammatory acne lesions compared to normal skin. In some embodiments, the gene is, or an expression product expressed by, a gene selected from the group consisting of defensin beta 4 (DEFB4), S100 calcium binding protein A8 (calgranulin B) (S100A8), Transcribed locus (GenBank Accession AI741601), SH3-domain GRB2-like 3 (GenBank Accession AK098337), cysteinyl leukotriene receptor 1 (CYSLTR1), ubiquitin-like modifier activating enzyme 6 (UBA6), programmed cell death 7 (PDCD7), Wilms tumor 1 associated protein (WTAP), aprataxin (APTX), serpin peptidase inhibitor clade B (ovalbumin) member 3 (SERPINB3), solute carrier family 16 member 10 (SLC16A10), interleukin 8 (IL-8), cystatin E/M (CST6), serine peptidase inhibitor Kazal type 5 (SPINK5), TIMP metallopeptidase inhibitor 3 (TIMP3), caspase recruitment domain family, member 18 (CARD18), serpin peptidase inhibitor, clade A (alpha-1 antiproteinase, andtitrypsin) member 3 (SERPINA3), WAP four-disulfide core domain 3 (WFCD3), cysteine rich transmembrane BMP regulator 1 (chordin-like) (CRIM1), serine peptidase inhibitor Kunitz type 2 (SPINT2), and combinations thereof. In some embodiments, the gene is, or in the case of an expression product, the expression product is expressed by, a gene selected from the group consisting of S100 calcium binding protein A8 (calgranulin B) (S100A8), Transcribed locus (GenBank Accession AI741601), SH3-domain GRB2-like 3 (GenBank Accession AK098337), cysteinyl leukotriene receptor 1 (CYSLTR1), ubiquitin-like modifier activating enzyme 6 (UBA6), programmed cell death 7 (PDCD7), Wilms tumor 1 associated protein (WTAP), aprataxin (APTX), serpin peptidase inhibitor clade B (ovalbumin) member 3 (SERPINB3), solute carrier family 16 member 10 (SLC16A10), cystatin E/M (CST6), serine peptidase inhibitor Kazal type 5 (SPINK5), TIMP metallopeptidase inhibitor 3 (TIMP3), caspase recruitment domain family, member 18 (CARD18), serpin peptidase inhibitor, clade A (alpha-1 antiproteinase, andtitrypsin) member 3 (SERPINA3), WAP four-disulfide core domain 3 (WFCD3), cysteine rich transmembrane BMP regulator 1 (chordin-like) (CRIM1), serine peptidase inhibitor Kunitz type 2 (SPINT2), and combinations thereof. In some embodiments, the gene or expression product is isolated, purified, or both. In some embodiments, the methods further comprise detecting a relative amount of the gene or gene product compared to a control. In some embodiments, the gene or gene expression product is a nucleic acid molecule or, in the case of a gene expression product, a protein. In some embodiments, the nucleic acid molecule is an RNA molecule. In some embodiments, the control is the relative amount of the gene or gene product expressed in a normal epidermal skin sample. In some embodiments, the relative amount of the gene or gene product is decreased compared to the control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the relative amount of the gene or gene product is increased compared to the control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the methods further comprise detecting the relative amount of two or more genes or gene products expressed by one or more genes listed in any of Tables 3, 6, 7, 8, or 9. In some embodiments, the methods further comprise detecting the relative amount of two, 3 or more, 4 or more, 5 or more, 6 or more, 7 or more, 8 or more, 9 or more, or 10 or more genes or gene products expressed by one or more genes listed in any of Tables 3, 6, 7, 8, or 9. In some embodiments, the methods further comprise detecting the relative amount of two, 3, 4, 5, 6, 7, 8, 9, or 10 genes or gene products expressed by one or more genes listed in any of Tables 3, 6, 7, 8, or 9. In some embodiments, the methods further comprise detecting the relative amount of 2, 3, 4, 5, 6, 7, 8, 9, 10 or more genes or gene products expressed by one or more genes listed in Table 3. In some embodiments, the methods further comprise detecting the relative amount of 2, 3, 4, 5, 6, 7, 8, 9, 10 or more gene or gene products expressed by one or more genes listed in Table 6. In some embodiments, the methods further comprise detecting the relative amount of 2, 3, 4, 5, 6, 7, 8, 9, 10 or more genes or gene products expressed by one or more genes listed in Table 7. In some embodiments, the methods further comprise detecting the relative amount of 2, 3, 4, 5, 6, 7, 8, 9, 10 or more genes or gene products expressed by one or more genes listed in Table 8. In some embodiments, the methods further comprise detecting the relative amount of 2, 3, 4, 5, 6, 7, 8, 9, 10 or more genes or gene products expressed by one or more genes listed in Table 9. In some embodiments, the methods further comprise detecting the relative amount of a gene or product expressed by one or more genes listed in Table 9 and a gene or gene product expressed by one or more genes listed in any of Tables 3, 6, 7, 8. In some embodiments, the methods further comprise detecting the relative amount of a gene or gene product expressed by DEFB4 and a gene or gene product expressed by one or more genes listed in Table 9. In some embodiments, the methods further comprise detecting the relative amount of a gene or gene product expressed by TIMP3 and a gene product expressed by one or more genes listed in Table 9. In some embodiments, the methods further comprise detecting the relative amount of a gene or gene product expressed by IL8 and a gene or gene product expressed by one or more genes listed in Table 9. In some embodiments, the methods further comprise isolating the nucleic acid from the epidermal sample. In some embodiments, the methods further comprise applying the isolated nucleic acid molecule to a microarray. In some embodiments, the methods further comprise amplifying the nucleic acid molecule from the sample prior to detecting. In some embodiments, the methods further comprise applying the amplification product thereof to a microarray. In some embodiments, detecting comprises measuring the amount of the nucleic acid hybridized to the microarray. In some embodiments, detecting comprises quantitative polymerase chain reaction. In some embodiments, 1, 2, 3, 4, 5, 6, 7, 8, 9, or 10 adhesive tapes are applied and removed from the skin. In some embodiments, 1, 2, 3, 4, 5, 6, 7, 8, 9, or 10 applications of a tape are applied and removed from the skin. In some embodiments, 1, 2, 3, 4, 5, 6, 7, 8, 9, or 10 adhesive tapes are applied to the same site sequentially. In some embodiments, 1, 2, 3, 4, 5, 6, 7, 8, 9, or 10 adhesive tapes are applied to different sites. In some embodiments, the adhesive tape comprises a rubber adhesive on a polyurethane film. In some embodiments, the method comprises applying the adhesive tape to the skin of the face, upper back, or upper chest of the subject. In some embodiments, the methods further comprise using the characterization to determine a treatment regimen. In some embodiments, the methods further comprise treating the subject for acne vulgaris. In some embodiments, the treatment comprises an antiseptic, an antibiotic, a retinoid, a hormone, an anti-inflammatory agent, an aldosterone receptor antagonist, comedo extraction, surgery, dermabrasion, or phototherapy. In some embodiments, the treatment comprises benzoyl peroxide, asapalene, azalaic acid, clindamycin, cephalexin, dapsone, dropirenone, doxycycline, erythromycin, ethinyl estradiol, isotretinoin, magnesium hydroxide, minocycline, salicylic acid, sodium sulfacetamide, sulfamethoxazole, spironolactone, tazarotene, tretinoin or trimethoprim. In some embodiments, the treatment is administered orally or is applied topically to the skin. In some embodiments, the suspected acne lesion is an inflammatory acne lesion or a non-inflammatory acne lesion. In some embodiments, the subject is a human. In some embodiments, the methods further comprise obtaining multiple epidermal samples over a period of time. In some embodiments, the method comprises monitoring the expression of the gene product over the time period. In some embodiments, the methods further comprise obtaining an epidermal sample from the subject prior to and following administration of a treatment for acne vulgaris. In some embodiments, the methods further comprise determining the difference in expression of the gene product between the epidermal sample obtained prior to treatment and the epidermal sample obtained following treatment. In some embodiments, the methods further comprise modifying the treatment based on the expression of the gene product following treatment. In some embodiments, the methods further comprise modifying the frequency of administration of the treatment. In some embodiments, the methods further comprise modifying the amount of the treatment administered.

Described herein, in certain embodiments, are methods for characterizing sensitivity of a subject to developing acne lesions comprising: applying an adhesive tape to a target area of the skin of the subject in a manner sufficient to isolate an epidermal sample adhering to the adhesive tape, wherein the epidermal sample comprises one or more genes, or gene product expressed by one or more genes, listed in any of Tables 3, 6, 7, 8, or 9; and characterizing the subject as having sensitivity to developing acne lesions based on the gene product present in the epidermal sample. In some embodiments, the gene is listed, or the expression product is expressed by a gene listed, on Table 3. In some embodiments, the gene is listed, or in the case of a gene product, the gene product is expressed by a gene listed, on Table 6. In some embodiments, the gene or gene product is listed on Table 7. In some embodiments, the gene or gene product is from Table 8. In some embodiments, the gene or gene product is listed on Table 9. In some embodiments, the gene or gene product is one that has increased expression in inflammatory acne lesions compared to normal skin. In some embodiments, the gene or gene product is one that has decreased expression in inflammatory acne lesions compared to normal skin. In some embodiments, the gene is, or the gene product is expressed by, a gene selected from the group consisting of defensin beta 4 (DEFB4), S100 calcium binding protein A8 (calgranulin B) (S100A8), Transcribed locus, SH3-domain GRB2-like 3, cysteinyl leukotriene receptor 1 (CYSLTR1), hypothetical protein FLJ10808, programmed cell death 7 (PDCD7), Wilms tumor 1 associated protein (WTAP), aprataxin (APTX), serpin peptidase inhibitor Glade B (ovalbumin) member 3 (SERPINB3), solute carrier family 16 member 10 (SLC16A10), interleukin 8 (IL-8), cystatin E/M (CST6), serine peptidase inhibitor Kazal type 5 (SPINK5), TIMP metallopeptidase inhibitor 3 (TIMP3), ICEBERG caspase-1 inhibitor, serpin peptidase inhibitor, Glade A (alpha-1 antiproteinase, andtitrypsin) member 3 (SERPINA3), WAP four-disulfide core domain 3 (WFCD3), cysteine rich transmembrane BMP regulator 1 (chordin-like) (CRIM1), serine peptidase inhibitor Kunitz type 2 (SPINT2), and combinations thereof. In some embodiments, the gene is, or the gene product is expressed by, a gene selected from the group consisting of S100 calcium binding protein A8 (calgranulin B) (S100A8), Transcribed locus (GenBank Accession AI741601), SH3-domain GRB2-like 3 (GenBank Accession AK098337), cysteinyl leukotriene receptor 1 (CYSLTR1), ubiquitin-like modifier activating enzyme 6 (UBA6), programmed cell death 7 (PDCD7), Wilms tumor 1 associated protein (WTAP), aprataxin (APTX), serpin peptidase inhibitor clade B (ovalbumin) member 3 (SERPINB3), solute carrier family 16 member 10 (SLC16A10), cystatin E/M (CST6), serine peptidase inhibitor Kazal type 5 (SPINK5), TIMP metallopeptidase inhibitor 3 (TIMP3), caspase recruitment domain family, member 18 (CARD18), serpin peptidase inhibitor, clade A (alpha-1 antiproteinase, andtitrypsin) member 3 (SERPINA3), WAP four-disulfide core domain 3 (WFCD3), cysteine rich transmembrane BMP regulator 1 (chordin-like) (CRIM1), serine peptidase inhibitor Kunitz type 2 (SPINT2), and combinations thereof. In some embodiments, the gene or expression product is isolated, purified, or both. In some embodiments, the methods further comprise detecting a relative amount of the gene or gene product compared to a control. In some embodiments, the gene or gene product is a nucleic acid molecule or a protein (optionally in the case of a gene product). In some embodiments, the nucleic acid molecule is an RNA molecule. In some embodiments, the control is the relative amount of the gene or gene product expressed in a normal epidermal skin sample. In some embodiments, the relative amount of the gene or the gene product is decreased compared to the control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the relative amount of the gene or gene product is increased compared to the control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the relative amount of two or more genes or gene products expressed by one or more genes listed in Tables 3, 6, 7, 8, or 9 are detected. In some embodiments, the methods further comprise detecting the relative amount of two or more genes or gene products expressed by one or more genes listed in any of Tables 3, 6, 7, 8, or 9. In some embodiments, the methods further comprise detecting the relative amount of two, 3 or more, 4 or more, 5 or more, 6 or more, 7 or more, 8 or more, 9 or more, or 10 or more genes or gene products expressed by one or more genes listed in any of Tables 3, 6, 7, 8, or 9. In some embodiments, the methods further comprise detecting the relative amount of two, 3, 4, 5, 6, 7, 8, 9, or 10 genes or gene products expressed by one or more genes listed in any of Tables 3, 6, 7, 8, or 9. In some embodiments, the methods further comprise detecting the relative amount of 2, 3, 4, 5, 6, 7, 8, 9, 10 or more genes or gene products expressed by one or more genes listed in Table 3. In some embodiments, the methods further comprise detecting the relative amount of 2, 3, 4, 5, 6, 7, 8, 9, 10 or more genes or gene products expressed by one or more genes listed in Table 6. In some embodiments, the methods further comprise detecting the relative amount of 2, 3, 4, 5, 6, 7, 8, 9, 10 or more genes or gene products expressed by one or more genes listed in Table 7. In some embodiments, the methods further comprise detecting the relative amount of 2, 3, 4, 5, 6, 7, 8, 9, 10 or more genes or gene products expressed by one or more genes listed in Table 8. In some embodiments, the methods further comprise detecting the relative amount of 2, 3, 4, 5, 6, 7, 8, 9, 10 or more genes or gene products expressed by one or more genes listed in Table 9. In some embodiments, the methods further comprise detecting the relative amount of a gene or gene product expressed by one or more genes listed in Table 9 and a gene or gene product expressed by one or more genes listed in any of Tables 3, 6, 7, 8. In some embodiments, the methods further comprise detecting the relative amount of a gene or gene product expressed by DEFB4 and a gene or gene product expressed by one or more genes listed in Table 9. In some embodiments, the methods further comprise detecting the relative amount of a gene or gene product expressed by DEFB4 and a gene or gene product expressed by one or more genes listed in Table 9. In some embodiments, the methods further comprise detecting the relative amount of a gene or gene product expressed by IL8 and a gene or gene product expressed by one or more genes listed in Table 9. In some embodiments, the gene or expression product is isolated, purified, or both. In some embodiments, the methods further comprise isolating the nucleic acid from the epidermal sample. In some embodiments, the methods further comprise applying the isolated nucleic acid molecule to a microarray. In some embodiments, the methods further comprise amplifying the nucleic acid molecule from the sample prior to detecting. In some embodiments, the methods further comprise applying the amplification product thereof to a microarray. In some embodiments, detecting comprises measuring the amount of the nucleic acid hybridized to the microarray. In some embodiments, detecting comprises quantitative polymerase chain reaction. In some embodiments, 1, 2, 3, 4, 5, 6, 7, 8, 9, or 10 adhesive tapes are applied and removed from the skin. In some embodiments, 1, 2, 3, 4, 5, 6, 7, 8, 9, or 10 applications of a tape are applied and removed from the skin. In some embodiments, 1, 2, 3, 4, 5, 6, 7, 8, 9, or 10 adhesive tapes are applied to the same site sequentially. In some embodiments, 1, 2, 3, 4, 5, 6, 7, 8, 9, or 10 adhesive tapes are applied to different sites. In some embodiments, the adhesive tape comprises a rubber adhesive on a polyurethane film. In some embodiments, the methods comprise applying the adhesive tape to the skin of the face, upper back, or upper chest of the subject. In some embodiments, the methods further comprise using the characterization to determine a treatment regimen. In some embodiments, the methods further comprise treating the subject for acne vulgaris. In some embodiments, the treatment comprises an antiseptic, an antibiotic, a retinoid, a hormone, an anti-inflammatory agent, an aldosterone receptor antagonist, comedo extraction, surgery, dermabrasion, or phototherapy. In some embodiments, the treatment comprises benzoyl peroxide, asapalene, azalaic acid, clindamycin, cephalexin, dapsone, dropirenone, doxycycline, erythromycin. ethinyl estradiol, isotretinoin, magnesium hydroxide, minocycline, salicylic acid, sodium sulfacetamide, sulfamethoxazole, spironolactone, tazarotene, tretinoin or trimethoprim. In some embodiments, the treatment is administered orally or is applied topically to the skin. In some embodiments, the subject is a mammal or human. In some embodiments, the methods further comprise obtaining multiple epidermal samples over a period of time. In some embodiments, the methods comprise monitoring the expression of the gene or gene product over the time period. In some embodiments, the methods further comprise obtaining an epidermal sample from the subject prior to and following administration of a treatment for acne vulgaris. In some embodiments, the methods further comprise determining the difference in expression of the gene or gene product between the epidermal sample obtained prior to treatment and the epidermal sample obtained following treatment. In some embodiments, the methods further comprise modifying the treatment based on the expression of the gene or gene product following treatment. In some embodiments, the methods further comprise modifying the frequency of administration of the treatment. In some embodiments, the methods further comprise modifying the amount of the treatment administered.

Described herein, in certain embodiments, are cosmetic formulations containing one or more agents for decreasing or increasing the expression of one or more genes listed in any of Tables 3, 6, 7, 8, or 9. In some embodiments, the cosmetic formulation comprises an emulsion, a cream, a lotion, a solution, an anhydrous base, a paste, a powder, a gel, or an ointment.

Described herein, in certain embodiments, are methods of treating acne vulgaris comprising administering the cosmetic formulation provided herein containing one or more agents for decreasing or increasing the expression of one or more genes listed in any of Tables 3, 6, 7, 8, or 9.

Described herein, in certain embodiments, are kits for determining a response of a subject to treatment for acne vulgaris comprising a skin sample collection device and one or more probes or primers that selectively bind to a gene of, or gene product expressed by, one or more genes listed in any of Tables 3, 6, 7, 8, or 9. In some embodiments, the gene or gene product is a nucleic acid molecule or a protein. In some embodiments, the nucleic acid molecule is an RNA molecule. In some embodiments, the skin sample collection device is an adhesive tape. In some embodiments, the adhesive tape comprises a rubber adhesive on a polyurethane film. In some embodiments, the one or more probes or primers are detectably labeled.

Described herein, in certain embodiments, are microarrays comprising one or more nucleic acids that selectively bind to a gene product expressed by one or more genes listed in any of Tables 3, 6, 7, 8, or 9.

Described herein, in certain embodiments are microarrays comprising one or more polypeptides that selectively bind to a gene or gene product expressed by a gene expressed by one or more genes listed in any of Tables 3, 6, 7, 8, or 9.

Described herein, in certain embodiments, are methods for screening a test compound for treatment of acne vulgaris comprising: contacting a skin cell culture in vitro with the test compound; and detecting the relative amount of the one or more genes or gene products expressed by one or more genes listed in any of Tables 3, 6, 7, 8, or 9 in the skin cell culture compared to a control; and characterizing the test compound as a drug candidate for treatment of acne vulgaris. In some embodiments, the gene is, or gene product is expressed by, a gene selected from Table 3. In some embodiments, the gene is, or the gene product is expressed by, a gene selected from Table 6. In some embodiments, the gene is, or the gene product is expressed by, a gene selected from Table 7. In some embodiments, the gene is, or the gene product is expressed by, a gene selected from Table 8. In some embodiments, the gene is, or the gene product is expressed by, a gene selected from Table 9. In some embodiments, the gene is, or the gene product is expressed by, one that has increased expression in inflammatory acne lesions compared to normal skin. In some embodiments, the gene is, or the gene product is one that has decreased expression in inflammatory acne lesions compared to normal skin. In some embodiments, the gene is, or the gene product is expressed by, a gene selected from the group consisting of defensin beta 4 (DEFB4), S100 calcium binding protein A8 (calgranulin B) (S100A8), Transcribed locus, SH3-domain GRB2-like 3, cysteinyl leukotriene receptor 1 (CYSLTR1), hypothetical protein FLJ10808, programmed cell death 7 (PDCD7), Wilms tumor 1 associated protein (WTAP), aprataxin (APTX), serpin peptidase inhibitor clade B (ovalbumin) member 3 (SERPINB3), solute carrier family 16 member 10 (SLC16A10), interleukin 8 (IL-8), cystatin E/M (CST6), serine peptidase inhibitor Kazal type 5 (SPINK5), TIMP metallopeptidase inhibitor 3 (TIMP3), ICEBERG caspase-1 inhibitor, serpin peptidase inhibitor, clade A (alpha-1 antiproteinase, andtitrypsin) member 3 (SERPINA3), WAP four-disulfide core domain 3 (WFCD3), cysteine rich transmembrane BMP regulator 1 (chordin-like) (CRIM1), serine peptidase inhibitor Kunitz type 2 (SPINT2), and combinations thereof. In some embodiments, the gene is, or the gene product is expressed by, a gene selected from the group consisting of S100 calcium binding protein A8 (calgranulin B) (S100A8), Transcribed locus (GenBank Accession AI741601), SH3-domain GRB2-like 3 (GenBank Accession AK098337), cysteinyl leukotriene receptor 1 (CYSLTR1), ubiquitin-like modifier activating enzyme 6 (UBA6), programmed cell death 7 (PDCD7), Wilms tumor 1 associated protein (WTAP), aprataxin (APTX), serpin peptidase inhibitor clade B (ovalbumin) member 3 (SERPINB3), solute carrier family 16 member 10 (SLC16A10), cystatin E/M (CST6), serine peptidase inhibitor Kazal type 5 (SPINK5), TIMP metallopeptidase inhibitor 3 (TIMP3), caspase recruitment domain family, member 18 (CARD18), serpin peptidase inhibitor, Glade A (alpha-1 antiproteinase, andtitrypsin) member 3 (SERPINA3), WAP four-disulfide core domain 3 (WFCD3), cysteine rich transmembrane BMP regulator 1 (chordin-like) (CRIM1), serine peptidase inhibitor Kunitz type 2 (SPINT2), and combinations thereof. In some embodiments, the gene or expression product is isolated, purified, or both. In some embodiments, the methods further comprise detecting a relative amount of a gene or gene product compared to a control. In some embodiments, the gene product is a nucleic acid molecule or a protein. In some embodiments, the nucleic acid molecule is an RNA molecule. In some embodiments, the control is the relative amount of the gene product expressed in an untreated skin cell culture. In some embodiments, the skin cell culture is a primary skin cell culture or a cell line. In some embodiments, the skin cell culture is a human epidermal skin cell culture.

Described herein, in certain embodiments, are methods for treating acne vulgaris comprising administering the drug candidate identified by the screening methods provided herein. In some embodiments, the relative amount of the gene or gene product is decreased compared to the control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the relative amount of the gene product is increased compared to the control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the relative amount of two or more genes, or gene products expressed by one or more genes, listed in Tables 3, 6, 7, 8, or 9 are detected. In some embodiments, the methods further comprise detecting the relative amount of two or more genes or gene products expressed by one or more genes listed in any of Tables 3, 6, 7, 8, or 9. In some embodiments, the methods further comprise detecting the relative amount of two, 3 or more, 4 or more, 5 or more, 6 or more, 7 or more, 8 or more, 9 or more, or 10 or more genes or gene products expressed by one or more genes listed in any of Tables 3, 6, 7, 8, or 9. In some embodiments, the methods further comprise detecting the relative amount of two, 3, 4, 5, 6, 7, 8, 9, or 10 genes or gene products expressed by one or more genes listed in any of Tables 3, 6, 7, 8, or 9. In some embodiments, the methods further comprise detecting the relative amount of 2, 3, 4, 5, 6, 7, 8, 9, 10 or more genes or gene products expressed by one or more genes listed in Table 3. In some embodiments, the methods further comprise detecting the relative amount of 2, 3, 4, 5, 6, 7, 8, 9, 10 or more genes or gene products expressed by one or more genes listed in Table 6. In some embodiments, the methods further comprise detecting the relative amount of 2, 3, 4, 5, 6, 7, 8, 9, 10 or more genes or gene products expressed by one or more genes listed in Table 7. In some embodiments, the methods further comprise detecting the relative amount of 2, 3, 4, 5, 6, 7, 8, 9, 10 or more genes or gene products expressed by one or more genes listed in Table 8. In some embodiments, the methods further comprise detecting the relative amount of 2, 3, 4, 5, 6, 7, 8, 9, 10 or more genes or gene products expressed by one or more genes listed in Table 9. In some embodiments, the methods further comprise detecting the relative amount of a gene or gene product expressed by one or more genes listed in Table 9 and a gene or gene product expressed by one or more genes listed in any of Tables 3, 6, 7, 8. In some embodiments, the methods further comprise detecting the relative amount of a gene or gene product expressed by DEFB4 and a gene or gene product expressed by one or more genes listed in Table 9. In some embodiments, the methods further comprise detecting the relative amount of a gene or gene product expressed by TIMP3 and a gene or gene product expressed by one or more genes listed in Table 9. In some embodiments, the methods further comprise detecting the relative amount of a gene or gene product expressed by IL8 and a gene product expressed by one or more genes listed in Table 9. In some embodiments, the methods further comprise isolating the nucleic acid from the skin cell culture. In some embodiments, the methods further comprise applying the isolated nucleic acid molecule to a microarray. In some embodiments, the methods further comprise amplifying the nucleic acid molecule from the skin cell culture prior to detecting. In some embodiments, the methods further comprise applying the amplification product thereof to a microarray. In some embodiments, detecting comprises measuring the amount of the nucleic acid hybridized to the microarray. In some embodiments, detecting comprises quantitative polymerase chain reaction.

Described herein, in certain embodiments, are adhesive tapes comprising an epidermal sample of an acne lesion that comprises a gene or gene product expressed by one or more genes in any of Tables 3, 6, 7, 8, or 9, wherein the epidermal sample is of a sufficient quantity to allow determination of the relative amount of a gene or gene product present in the epidermal sample. In some embodiments, the gene or gene product is expressed by a gene is from Table 3. In some embodiments, the gene or gene product is expressed by a gene is from Table 6. In some embodiments, the gene or gene product is expressed by a gene is from Table 7. In some embodiments, the gene or gene product is expressed by a gene is from Table 8. In some embodiments, the gene or gene product is expressed by a gene is from Table 9. In some embodiments, the gene or gene product is one that has increased expression in inflammatory acne lesions compared to normal skin. In some embodiments, the gene or gene product is one that has decreased expression in inflammatory acne lesions compared to normal skin. In some embodiments, the gene or gene product is expressed by a gene selected from the group consisting of defensin beta 4 (DEFB4), S100 calcium binding protein A8 (calgranulin B) (S100A8), Transcribed locus, SH3-domain GRB2-like 3, cysteinyl leukotriene receptor 1 (CYSLTR1), hypothetical protein FLJ10808, programmed cell death 7 (PDCD7), Wilms tumor 1 associated protein (WTAP), aprataxin (APTX), serpin peptidase inhibitor clade B (ovalbumin) member 3 (SERPINB3), solute carrier family 16 member 10 (SLC16A10), interleukin 8 (IL-8), cystatin E/M (CST6), serine peptidase inhibitor Kazal type 5 (SPINK5), TIMP metallopeptidase inhibitor 3 (TIMP3), ICEBERG caspase-1 inhibitor, serpin peptidase inhibitor, clade A (alpha-1 antiproteinase, andtitrypsin) member 3 (SERPINA3), WAP four-disulfide core domain 3 (WFCD3), cysteine rich transmembrane BMP regulator 1 (chordin-like) (CRIM1), serine peptidase inhibitor Kunitz type 2 (SPINT2), and combinations thereof. In some embodiments, the gene or gene product expressed by a gene is from the group consisting of S100 calcium binding protein A8 (calgranulin B) (S100A8), Transcribed locus (GenBank Accession AI741601), SH3-domain GRB2-like 3 (GenBank Accession AK098337), cysteinyl leukotriene receptor 1 (CYSLTR1), ubiquitin-like modifier activating enzyme 6 (UBA6), programmed cell death 7 (PDCD7), Wilms tumor 1 associated protein (WTAP), aprataxin (APTX), serpin peptidase inhibitor clade B (ovalbumin) member 3 (SERPINB3), solute carrier family 16 member 10 (SLC16A10), cystatin E/M (CST6), serine peptidase inhibitor Kazal type 5 (SPINK5), TIMP metallopeptidase inhibitor 3 (TIMP3), caspase recruitment domain family, member 18 (CARD18), serpin peptidase inhibitor, clade A (alpha-1 antiproteinase, andtitrypsin) member 3 (SERPINA3), WAP four-disulfide core domain 3 (WFCD3), cysteine rich transmembrane BMP regulator 1 (chordin-like) (CRIM1), serine peptidase inhibitor Kunitz type 2 (SPINT2), and combinations thereof. In some embodiments, the gene or expression product is isolated, purified, or both. In some embodiments, the gene or gene product is a nucleic acid molecule or a protein. In some embodiments, the nucleic acid molecule is an RNA molecule. In some embodiments, the adhesive tape comprises a rubber adhesive on a polyurethane film. In some embodiments, the epidermal sample is from the skin of the face, upper back, or upper chest of the subject.

### BRIEF DESCRIPTION OF THE DRAWINGS

FIG. 1A and FIG. 1B illustrate the relative expression of Tissue inhibitor of metalloproteinase 3 (TIMP 3) in normal skin (NS), in inflammatory acne lesions (IN) at day 1 prior to Duac treatment, and in inflammatory acne lesions (IN) at weeks 2, week 5 and week eight post-Duac treatment (1.2% clindamycin phosphate, 5% benzoyl peroxide gel). TIMP3 expression is decreased in inflammatory acne lesions compared to normal skin and increases over the course of Duac treatment. Diagram of TIMP3 signaling pathways and effects of TIMP3 inhibition.

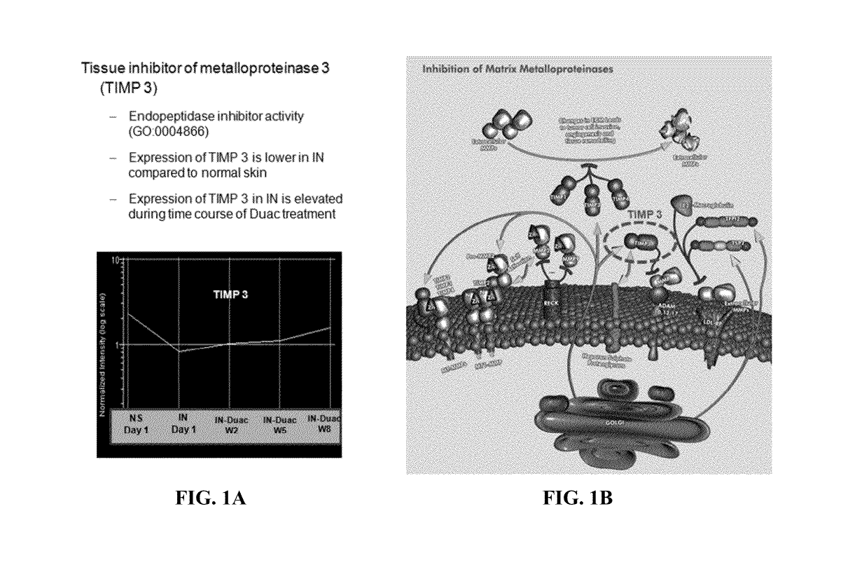

FIG. 2: illustrates expression of Defensin (34 in normal skin (NS), in inflammatory acne lesions (IN) at day 1 prior to Duac treatment, and in inflammatory acne lesions (IN) at weeks 2, week 5 and week eight post-Duac treatment. Defensin (34 expression is increased in inflammatory acne lesions compared to normal skin and decreases over the course of Duac treatment.

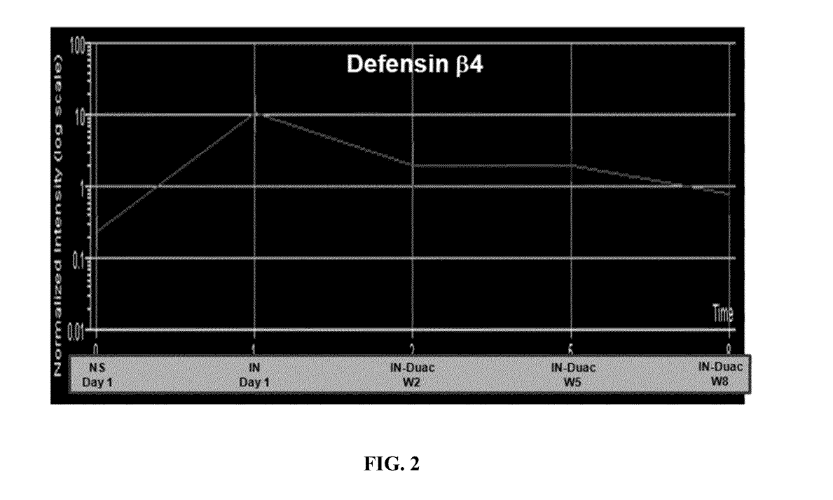

FIG. 3 illustrates the relative expression of gene according to a Self-Organizing Map analysis which groups genes with similar expression profiles into clusters. The graphs depict relative gene expression in normal skin (NS), in inflammatory acne lesions (IN) at day 1 prior to Duac treatment, and in inflammatory acne lesions (IN) at weeks 2, week 5 and week eight post-Duac treatment. 806 genes, which were differentially expressed in acne lesions compared to normal skin (Table 3) were subjected to SOM: 4×3 nodes with 10,000 iterations. 270 genes were over-expressed in inflammatory acne lesions compared to normal skin controls at Day 1, and exhibited decreased expression in inflammatory acne lesions during Duac treatment. 261 genes were under-expressed in inflammatory acne lesions compared to normal skin controls at Day 1, and exhibited increased expression in inflammatory acne lesions during Duac treatment.

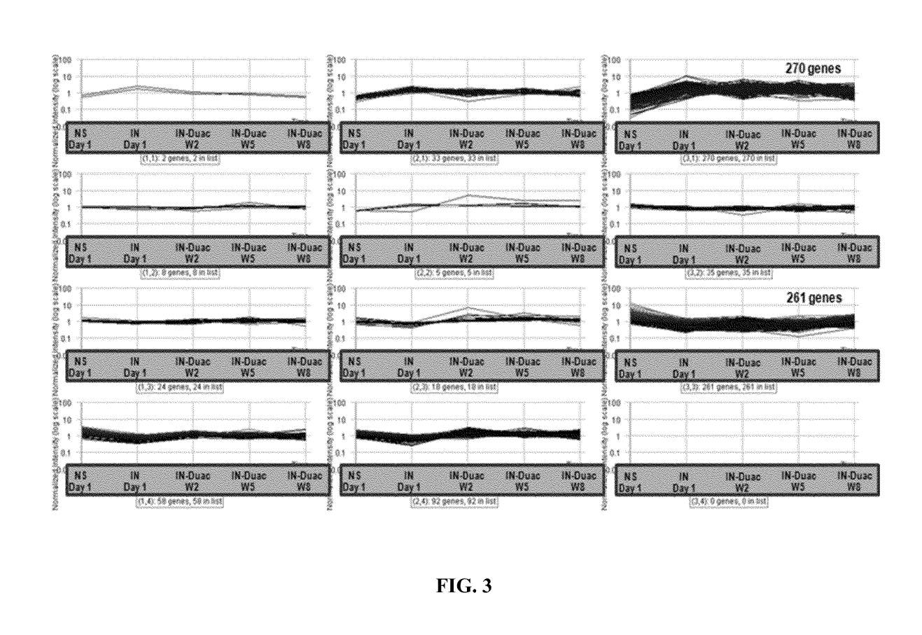

FIG. 4A and FIG. 4B illustrate relative gene expression in normal skin (NS), in inflammatory acne lesions (IN) at day 1 prior to Duac treatment, and in inflammatory acne lesions (IN) at weeks 2, week 5 and week eight post-Duac treatment for 270 genes that were over-expressed in inflammatory acne lesions compared to normal skin controls at Day 1, and exhibited decreased expression in inflammatory acne lesions during Duac treatment and a subset of 126 genes (Table 6) with most differential expression in inflammatory acne lesions between pre- and 8 weeks post-Duac treatment.

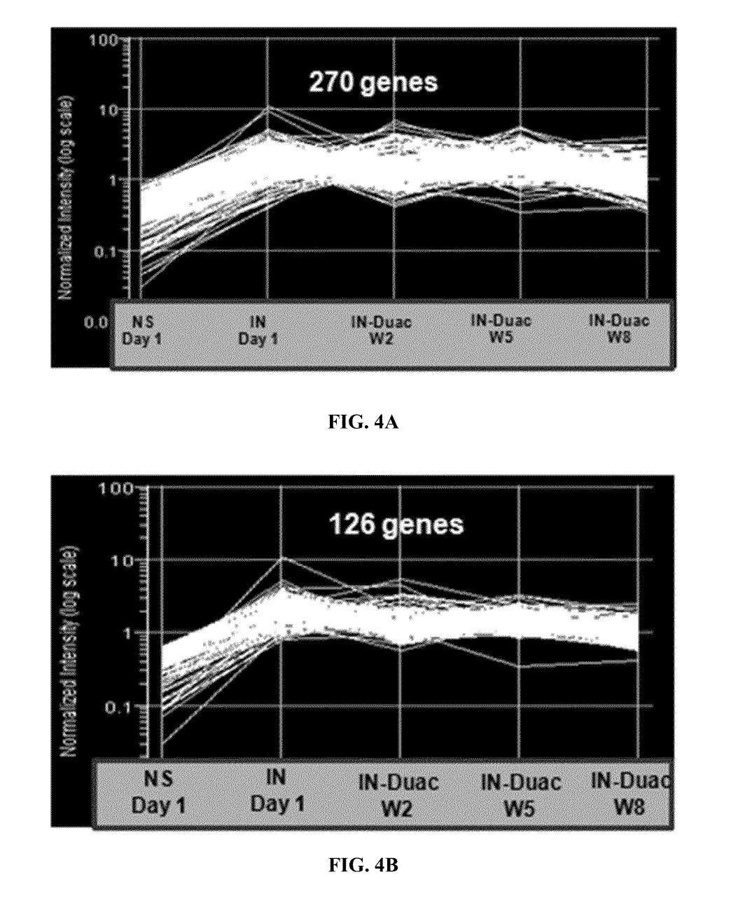

FIG. 5A and FIG. 5B: illustrate relative gene expression in normal skin (NS), in inflammatory acne lesions (IN) at day 1 prior to Duac treatment, and in inflammatory acne lesions (IN) at weeks 2, week 5 and week eight post-Duac treatment for 261 genes that were under-expressed in inflammatory acne lesions compared to normal skin controls at Day 1, and exhibited increased expression in inflammatory acne lesions during Duac treatment and a subset of 119 genes (Table 7) with most differential expression in inflammatory acne lesions between pre- and 8 weeks post-Duac treatment.

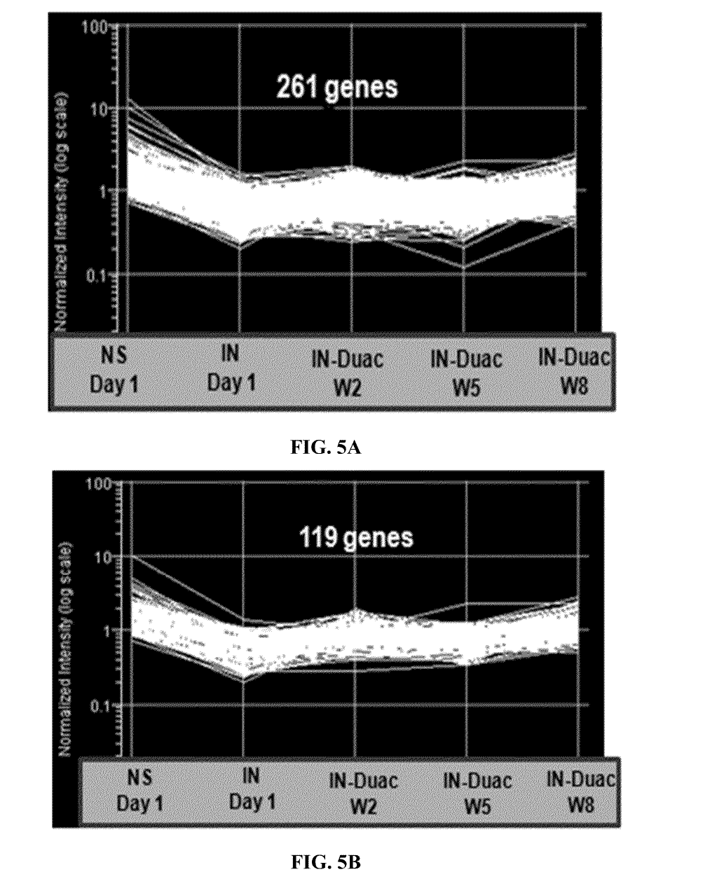

FIG. 6A and FIG. 6B illustrate relative gene expression in normal skin (NS), in inflammatory acne lesions (IN) at day 1 prior to Duac treatment, and in inflammatory acne lesions (IN) at weeks 2, week 5 and week eight post-Duac treatment for a subset of 12 genes (from 126) that were over-expressed in inflammatory acne lesion and expression was decreased during Duac treatment and a subset of 8 genes (from 119) that were under-expressed in inflammatory acne lesions and expression was increased during Duac treatment Table 8.

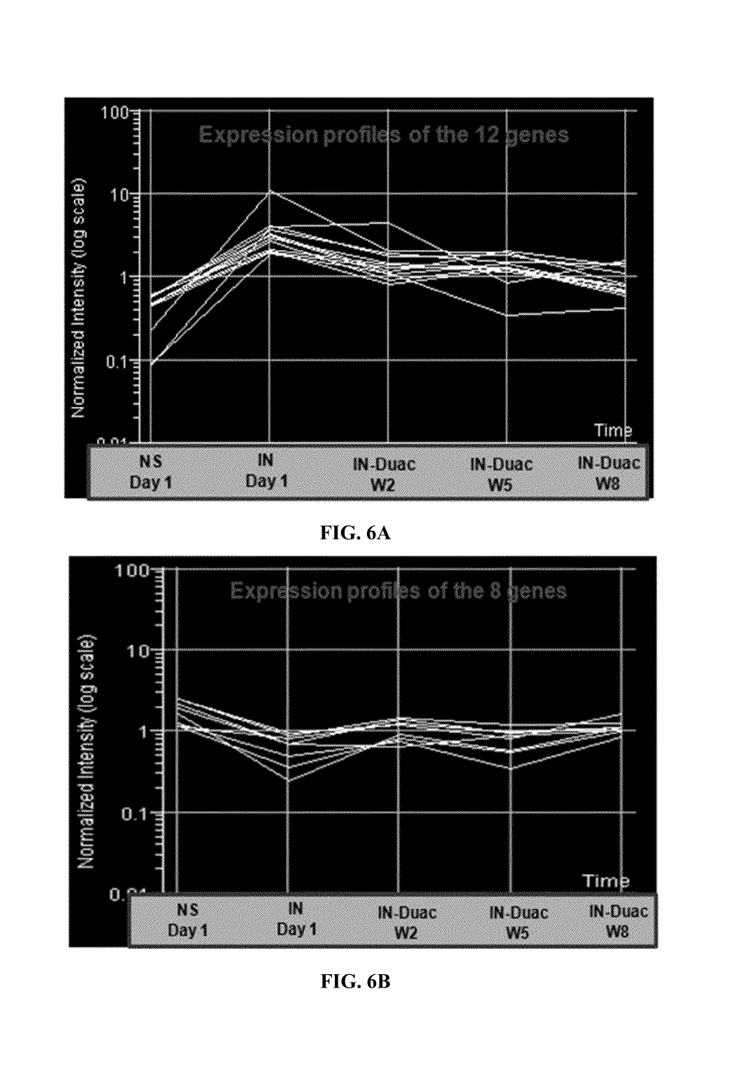

### DETAILED DESCRIPTION OF THE INVENTION

### Certain Terminology

Unless defined otherwise, all technical and scientific terms used herein have the same meaning as is commonly understood by one of skill in the art to which the claimed subject matter belongs. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information. Generally, the procedures for cell culture, cell infection, antibody production and molecular biology methods are methods commonly used in the art. Such standard techniques can be found, for example, in reference manual, such as, for example, Sambrook et al. (2000) and Ausubel et al. (1994).

As used herein, the singular forms “a,” “an” and “the” include plural referents unless the context clearly dictates otherwise. In this application, the use of the singular includes the plural unless specifically stated otherwise. As used herein, the use of “or” means “and/or” unless stated otherwise. Furthermore, use of the term “including” as well as other forms (e.g., “include”, “includes”, and “included”) is not limiting.

As used herein, ranges and amounts can be expressed as “about” a particular value or range. About also includes the exact amount. Hence “about 40 mg,” by way of non-limiting example only, means “about 40 mg” and also “40 mg.”

As used herein, “gene product” means any product expressed by a gene, including nucleic acids or polypeptides. In some embodiments, a gene product is a transcribed nucleic acid, such as an RNA. In some embodiments, the RNA is a coding RNA, e.g. a messenger RNA (mRNA). In some embodiments, the RNA is a non-coding RNA. In some embodiments, the non-coding RNA is a transfer RNA (tRNA), ribosomal RNA (rRNA), snoRNA, microRNA, siRNA, snRNA, exRNA, piRNA and long ncRNA. In some embodiments, the RNA is tRNA. In some embodiments, a gene product is a protein that is translated from and expressed mRNA. In some embodiments, each gene or expression product is present in an isolated form, a purified form, or both.

As used herein, the term “sample” refers to any preparation derived from tissue of a subject. In some embodiments, a sample of cells obtained using the non-invasive method described herein is used to isolate polynucleotides, polypeptides, metabolites, and/or lipids, for the methods provided herein. In some embodiments, samples for the methods provided herein are taken from a skin lesion, that is suspected of being the result of a disease or a pathological state, such as acne vulgaris. In some embodiments, samples are taken of the skin surface of the suspicious lesion using non-invasive skin sampling methods described herein.

As used herein, the term “skin” broadly refers to the outer protective covering of the body, consisting of the corium and the epidermis, and is understood to include sweat and sebaceous glands, as well as hair follicle structures. As used herein, the term “cutaneous” refers generally to attributes of the skin, as appropriate to the context in which they are used. In some embodiments, the skin is mammalian skin. In some embodiments, the skin is human skin.

As used herein, the term “acne vulgaris skin marker” or “acne vulgaris skin biomarker” is a gene whose expression level is different between skin samples at the site of an acne lesion and skin samples of uninvolved skin. Therefore, expression of an acne vulgaris skin marker is related to, or indicative of, acne vulgaris. As discussed herein, all of the acne vulgaris skin markers illustrated herein exhibit differential gene expression in an acne lesion versus non-acne lesion. In some embodiments, the acne vulgaris skin marker exhibits increased expression in an acne lesion compared to non-involved skin. In some embodiments, the acne vulgaris skin marker exhibits decreased expression in an acne lesion compared to non-involved skin. In some embodiments, methods provided herein, for example methods using microarrays to perform gene expression analysis using samples obtained from tape stripped skin, are used to identify additional acne vulgaris markers. The expression of these acne vulgaris makers can increase or decrease in acne lesions.

An “agent” as used herein is used broadly herein to mean any molecule to which skin is exposed. The term “test agent” or “test molecule” is used broadly herein to mean any agent that is being examined for an effect on skin in a method of the invention. For example, the agent can be a biomolecule or a small organic molecule. In illustrative examples, the agent is a peptide, polypeptide, or protein, a peptidomimetic, an oligosaccharide, a lipoprotein, a glycoprotein or glycolipid, a chemical, including, for example, a small organic molecule, which can be formulated as a drug or other pharmaceutical agent, or a nucleic acid, such as a polynucleotide.

As used herein, a “biologic” is a molecule derived from a living organism. Biologics used to treat acne vulgaris typically target precise immune or bacterial responses involved with acne vulgaris.

### Overview

The methods, systems, platforms, and kits provided herein relate to the identification and treatment of acne vulgaris. Acne vulgaris, also referred to as cystic acne or simply acne, is a common human skin disease that affects nearly all adolescents and adults at some time in their lives. In certain instances, acne vulgaris characterized by areas of skin with seborrhea (i.e. scaly red skin), comedones (e.g., blackheads and whiteheads), papules (e.g., pinheads), pustules (e.g., pimples), nodules (e.g., large papules) and possibly scarring. Acne affects mostly skin with the densest population of sebaceous follicles. These areas include the face, the upper part of the chest, and the back. Acne can manifest in inflammatory and noninflammatory forms.

Certain embodiments provided herein are based in part on the finding that samples from the epidermis of the skin, containing gene products, such as nucleic acid molecules, for example RNA, can be obtained from inflammatory acne lesions using a non-invasive tape stripping method in subjects having acne vulgaris. As described herein, the methods provided herein assist in, for example, identifying acne vulgaris in a subject, determining the severity of acne vulgaris, determining the sensitivity of a subject to developing acne vulgaris, determining the likelihood of a subject to respond to a therapy, selecting effective treatments for acne vulgaris, and monitoring of the efficacy of treatments for acne vulgaris. As described herein, the methods provided herein also assist in the screening of test agents for effective treatment of acne vulgaris. In some embodiments, the methods provided herein also assist in the screening of test agents that cause acne vulgaris or increase the sensitivity of a subject to the development of acne vulgaris.

The epidermis of the human skin comprises several distinct layers of skin tissue. The deepest layer is the stratum basalis layer, which consists of columnar cells. The overlying layer is the stratum spinosum, which is composed of polyhedral cells. Cells pushed up from the stratum spinosum are flattened and synthesize keratohyalin granules to form the stratum granulosum layer. As these cells move outward, they lose their nuclei, and the keratohyalin granules fuse and mingle with tonofibrils. This forms a clear layer called the stratum lucidum. The cells of the stratum lucidum are closely packed. As the cells move up from the stratum lucidum, they become compressed into many layers of opaque squamae. These cells are all flattened remnants of cells that have become completely filled with keratin and have lost all other internal structure, including nuclei. These squamae constitute the outer layer of the epidermis, the stratum corneum. At the bottom of the stratum corneum, the cells are closely compacted and adhere to each other strongly, but higher in the stratum they become loosely packed, and eventually flake away at the surface.

In certain embodiments, the skin sample obtained using the tape stripping method described herein includes epidermal cells, including cells comprising adnexal structures (e.g., vellus hair follicles and cells lining sebaceous, eccrine, and sweat ducts). In certain illustrative examples, the sample includes predominantly epidermal cells, or even exclusively epidermal cells. The epidermis consists predominantly of keratinocytes (>90%), which differentiate from the basal layer, moving outward through various layers having decreasing levels of cellular organization, to become the cornified cells of the stratum corneum layer. Renewal of the epidermis occurs every 20-30 days in uninvolved skin. Other cell types present in the epidermis include melanocytes, Langerhans cells, and Merkel cells. In certain embodiments, the tape stripping method described herein is particularly effective at isolating epidermal samples. In certain embodiments, the tape stripping method described herein is effective at isolating epidermal samples from acne lesions. In certain embodiments, the tape stripping method described herein is effective at isolating epidermal samples from acne lesions at any stage. In certain embodiments, the tape stripping method described herein is effective at isolating epidermal samples from acne lesions that are microcomedones (i.e. early stage acne lesion), seborrhea, comedones, papules, pustules, or nodules.

The methods, systems, platforms, and kits provided herein are based on a non-invasive approach for recovering or analyzing genes or gene products, such as nucleic acid molecule (e.g., DNA or RNA) and/or polypeptides, from the surface of skin via a simple tape stripping procedure that permits a direct quantitative and qualitative assessment of pathologic and physiologic biomarkers. Tape-harvested RNA is comparable in quality and utility to RNA recovered by biopsy. The present method causes little or no discomfort to the patient. Therefore, it can be performed routinely in a physician's office, for example, for point of care testing. Accordingly, provided herein are methods and markers for non-invasive isolation and/or detection of gene products, such as nucleic acid molecule and/or polypeptides from epidermal samples using tape stripping. In some embodiments, an epidermal sample is obtained from an acne lesion or a suspected acne lesion. In some embodiments, an epidermal sample is obtained from uninvolved skin. In some embodiments, epidermal sample obtained from uninvolved skin is compared to an epidermal sample obtained from an acne lesion or a suspected acne lesion. In some embodiments, epidermal sample obtained from an acne lesion that is an acne microcomedone, seborrhea, comedone, papule, pustule, or nodule. In some embodiments, epidermal sample obtained from an acne lesion is obtained prior to, during, or following administration of an acne treatment or therapeutic regimen.

In certain embodiments, the methods, systems, platforms, and kits include detecting expression of genes in the skin involves applying an adhesive tape to a target area of the skin in a manner sufficient to isolate an epidermal sample adhering to the adhesive tape, wherein the epidermal sample comprises a gene product. The genes and gene products in the epidermal sample are then detected. In some embodiments, gene or gene products are applied to a microarray to detect the gene or gene products. In some embodiments, the gene or gene product is isolated from the epidermal sample. In some embodiments, the gene or gene product is a nucleic acid molecule, such as an RNA or a DNA molecule. In some embodiments, nucleic acid is amplified. In some embodiments, the gene or gene product is a polypeptide.

Accordingly, non-invasive methods, systems, platforms, and kits are provided for isolating or detecting a gene or gene product, such as nucleic acid molecule from an epidermal sample of an acne lesion of a human subject, including applying an adhesive tape to the acne lesion of the subject in a manner sufficient to isolate an epidermal sample adhering to the adhesive tape. In some embodiments, the epidermal sample includes a nucleic acid molecule or a polypeptide that is then isolated and/or detected.

In some embodiments, the isolated nucleic acid encodes a protein such as a protein expressed by a gene of any of Tables 3, 6, 7, 8, or 9. In some embodiments, expression of these gene products are analyzed in acne lesions. The methods provided herein are useful, for example, for monitoring response to treatment for acne vulgaris; for determining a treatment that is likely most effective, for genetically characterizing acne vulgaris; for diagnosing acne vulgaris; and for identifying and analyzing nucleic acids that are predictive for response to a treatment for acne vulgaris. Changes in expression of genes listed in Tables 3, 6, 7, 8, or 9 is shown in the Examples provided herein to be associated with acne vulgaris. In some embodiments, expression of a gene listed in Table 3 is elevated in inflammatory acne lesions in patients with acne vulgaris. In some embodiments, expression of a gene listed in Table 3 is decreased in inflammatory acne lesions in patients with acne vulgaris. In some embodiments, expression of a gene listed in Table 6 is elevated in inflammatory acne lesions in patients with acne vulgaris. In some embodiments, expression of a gene listed in Table 6 is elevated in inflammatory acne lesions in patients with acne vulgaris, and decreases expression following treatment for acne vulgaris. In some embodiments, expression of a gene listed in Table 7 is decreased in inflammatory acne lesions in patients with acne vulgaris. In some embodiments, expression of a gene listed in Table 7 is decreased in inflammatory acne lesions in patients with acne vulgaris, and increases expression following treatment for acne vulgaris. Accordingly, in certain aspects, expression of genes listed in Tables 3, 6, 7, 8, or 9 is analyzed. In other aspects, expression of a subset of genes selected from any of Tables 3, 6, 7, 8, or 9 is analyzed. In other aspects, expression of the subset of genes listed in Table 3 is analyzed. In other aspects, expression of a subset of genes listed in Table 3 is analyzed. In other aspects, expression of the subset of genes listed in Table 6 is analyzed. In other aspects, expression of a subset of genes listed in Table 6 is analyzed. In other aspects, expression of the subset of genes listed in Table 7 is analyzed. In other aspects, expression of a subset of genes listed in Table 7 is analyzed. In other aspects, expression of the subset of genes listed in Table 8 is analyzed. In other aspects, expression of a subset of genes listed in Table 8 is analyzed. In other aspects, expression of the subset of genes listed in Table 9 is analyzed. In other aspects, expression of a subset of genes listed in Table 9 is analyzed.

Methods, systems, platforms, and kits provided herein which isolate and detect a nucleic acid sample from an epidermal sample of an acne lesion have utility not only in detecting acne vulgaris, but also in diagnosing, and prognosing acne vulgaris as well as monitoring response of a subject to treatment. In some embodiments, these methods are used to identify a predictive skin marker to identify an acne lesion and/or a patient, that will respond to treatment for acne vulgaris.

Biopsy and tape stripping methods are not equivalent sampling methods and do not yield identical gene expression results. Not intended to be limited by theory, tape stripping, also referred to as “tape harvesting,” is restricted to the skin surface and therefore may preferentially recover vellus hair follicles and cells lining sebaceous, eccrine and sweat ducts as well as corneocytes. Tape stripping methods provided herein, which typically utilize 10 or less tape strippings, for example, a single application of 4 individual tapes, do not result in glistening of uninvolved skin and thus do not bare the viable epidermis. Thus, tape stripping methods provided herein, provide an epidermal sample. In contrast, a shave biopsy, in which a scalpel blade is used to slice a thin piece of skin from the surface (and which typically results in bleeding but does not require suturing) or a punch biopsy, in which a circular blade is used to produces a cylindrical core of skin tissue 1 mm to 8 mm in length, are expected to include not only cells of the epidermis (primarily keratinocytes and melanocytes and immune cells) but fibroblasts from the upper dermis. Biopsy methods are invasive procedures that are risky and expensive to the patient, whereas the tape stripping method is non-invasive, safer, and less expensive than biopsy methods. The potential enrichment of surface epidermis conveyed by tape stripping compared to a shave or punch biopsy can be appreciated by considering that the surface area of a tape is 284 mm², while the surface area of a 2×2 mm shave biopsy is 4 mm². Thus, tape-harvested cells represent an enrichment of a sub-population of cells found in a shave or punch biopsy. In some embodiments, the tape stripped samples exhibit a differences in the gene expression profile compared to a biopsy method in acne lesions.

### Methods of Sample Collection

In some embodiments, an epidermal sample is obtained by tape stripping the skin. In some embodiments, tape stripping involves applying an adhesive tape to the skin in a manner sufficient to isolate an epidermal sample adhering to the tape. In some embodiments, the epidermal sample comprises a gene or gene product. In some embodiments, the gene product is a nucleic acid molecules and/or proteins. In some embodiments, the nucleic acid molecules are RNA molecules. In some embodiments, the RNA is mRNA.

Generally, before contacting a skin site with adhesive tape, a skin site to be stripped is cleaned, for example using an antiseptic cleanser such as alcohol. Next, tape is applied to a skin site with pressure. In some embodiments, pressure is applied for a fraction of a second. In some embodiments, pressure is applied for between about 1 second and about 5 minutes, for example, between about 10 seconds and about 45 seconds. In certain illustrative examples, the tape is applied with pressure for about 30 seconds for each tape stripping. It will be understood that the amount of pressure applied to a skin site and the length of time for stripping can be varied to identify ideal pressures and times for a particular application. Generally, pressure is applied by manually pressing down the adhesive tape on the skin. In some embodiments, objects, such as blunt, flat objects are used to assist in applying the tape to the skin, for example, in areas of the skin from which it is more difficult to obtain gene product samples from skin, such as uninvolved skin of a subject afflicted with acne vulgaris.

Virtually any size and/or shape of adhesive tape and target skin site size and shape can be used and analyzed, respectively, by the methods of the present invention. In some embodiments, adhesive tape is fabricated into circular discs of diameter between about 10 millimeters and about 100 millimeters, for example between about 15 millimeters and about 25 millimeters in diameter. In some embodiments, the adhesive tape has a surface area of between about 50 mm² and about 1000 mm², such as between about 100 mm² to about 500 mm², or about 250 mm².

In some embodiments, the tape stripping methods provided herein involve applying an adhesive tape to the skin of a subject and removing the adhesive tape from the skin of the subject one or more times. In certain examples, the adhesive tape is applied to the skin and removed from the skin about one to ten times. In some embodiments, an adhesive tape is applied to and removed from a target site 1, 2, 3, 4, 5, 6, 7, 8, 9, 10 or more times. In one illustrative example, the adhesive tape is applied to the skin and removed from the skin between about one and eight times. In one illustrative example, the adhesive tape is applied to the skin and removed from the skin between about one and five times. In one illustrative example, the adhesive tape is applied to the skin and removed from the skin.

In certain examples, about multiple adhesive tapes are applied to the skin and removed from the skin. In certain examples, about two to about ten adhesive tapes are applied to the skin and removed from the skin. In certain examples, multiple adhesive tapes are combined for further analysis. In some embodiments, 1, 2, 3, 4, 5, 6, 7, 8, 9, 10 or more adhesive tape(s) is/are applied to and removed from the target site. In some embodiments, multiple adhesive tapes are applied to the skin and removed from the skin multiple times.

In some embodiments, for the tape strippings, the same strip of tape is repeatedly applied to, and removed from, a target site, such as an acne lesion or a suspected acne lesion. In some embodiments, two or more fresh pieces of adhesive tape are sequentially applied to the same target site of the skin. In some embodiments, the individual tape strips used to sample a site are combined into one extraction vessel for further processing. In some embodiments, further processing involves isolation of a gene product from the sample. In some embodiments, further processing involves isolation of nucleic acid molecules and/or proteins from the sample.

In some embodiments, the tape stripping method used for obtaining a sample depends on factors such as, but not limited to, the flexibility, softness, and composition of the adhesive tape used, the time the tape is allowed to adhere to the skin before it is removed, the force applied to the tape as it is applied to the skin, the prevalence of a gene product being analyzed, the disease status of the skin, and patient-to-patient variability. In some embodiments, a particular tape stripping method is selected to ensure that sufficient gene products are present in the epidermal sample.

In some embodiments, a tape stripped sample comprises tissues that are restricted to the surface of skin. In some embodiments, a tape stripped sample preferentially recovers vellus hair follicles and cells lining sebaceous, eccrine, and sweat ducts (i.e., the adnexal structures associated with the stratum corneum and epidermis), as well as corneocytes. In some embodiments, tape stripping is stopped before viable epidermis is exposed by ceasing tape stripping before the tissue glistens (i.e., becomes shiny, appears “moistened” or reflective). The tape stripping method is thus generally considered a “noninvasive” method.

In some embodiments, tape stripping sufficient to isolate an epidermal sample is tape stripping that is performed on the skin in a sufficient manner to obtain a gene product sample. In some embodiments, tape stripping sufficient to isolate an epidermal sample is tape stripping that is performed on the skin a sufficient length of time to obtain a gene product sample. In some embodiments, tape stripping sufficient to isolate an epidermal sample is tape stripping that is performed on the skin a sufficient number of times to obtain a gene product sample. In some embodiments, tape stripping sufficient to isolate an epidermal sample is tape stripping that is performed on the skin a sufficient length of time over a sufficient number of times to obtain a gene product sample. In some embodiments, such tape stripping is stopped before the tissue glistens.

In certain embodiments, a conventional method, such as a skin biopsy is performed on the skin to obtain an additional skin sample. In some embodiments, the additional skin sample is obtained from uninvolved skin or involved skin, for example, an acne lesion or a suspected acne lesion. In some embodiments, uninvolved skin is skin that is not an acne lesion or is not suspected of being an acne lesion. In some embodiments, the additional skin sample provides additional information, for example, on expression of a gene product below the stratum corneum. In some embodiments, the additional skin sample is used for comparison against the skin samples obtained using the non-invasive methods provided herein. In some embodiments, the additional skin sample is employed for comparison to a tape stripped sample described herein.

As described herein, tape-harvested cells appear to represent an enrichment of a sub-population of cells found in a conventional skin sample, such as shave biopsy. Accordingly, in certain aspects, in addition to a tape stripping method provided herein, a biopsy can be taken at the site of tape stripping, such as an acne lesion site, or at another skin site. In some embodiments, the gene products from the biopsy are isolated and analyzed. In some embodiments, analysis of the biopsy data is combined with analysis of data from a tape stripping method to provide additional information regarding the acne lesion.

In some embodiments, a skin sample from uninvolved epidermal tissue is obtained. In some embodiments, the uninvolved skin sample is obtained by applying an adhesive tape to skin of the subject in a manner sufficient to isolate an epidermal sample adhering to the adhesive tape, wherein the epidermal sample includes gene products and wherein the skin is unaffected by the disease or condition to be tested. In some embodiments, the gene product is isolated and detected from the epidermal sample of the uninvolved skin. In some embodiments, the gene product is a nucleic acid molecule or a protein.

In some embodiments, the uninvolved skin is from the upper arm or the upper back. In certain embodiments, these sites appear to provide relatively plentiful quantities of nucleic acid molecules using tape strippings. For example, In some embodiments, tape stripping is performed on uninvolved skin over the deltoid or upper back over the scapular spine and the periauricular region. Tape stripping generally involves the skin surface. In some embodiments, tape stripping preferentially recovers vellus hair follicles and cells lining sebaceous, eccrine and sweat ducts (i.e. adnexal structures) as well as corneocytes (not predicted to contain RNA).

In some embodiments, skin samples obtained on adhesive films are frozen before being analyzed using the methods of provided herein. In some embodiments, freezing is performed by snap-freezing a sample using liquid nitrogen or dry ice.

In some embodiments, tape stripping is performed in a clinical setting by a first party that sends the tape strips to a second party for detection of the gene products, such as nucleic acid molecule or polypeptides. In some embodiments, the gene product is isolated from the epidermal sample. In some embodiments, gene product isolation is performed by either the first party or the second party. For example, in some embodiments, tape stripping is performed in a physician's office by a qualified practitioner, who sends the tape strips to a second party, such as an outside company who performs nucleic acid isolation and detection. Alternatively, nucleic acid isolation can be performed in the physician's office, who can send the isolated nucleic acid sample to a second party, such as an outside service provided, to perform nucleic acid detection and expression analysis.

In some embodiments, the subject is one having acne vulgaris or is suspected of having acne vulgaris. In some embodiments, the subject has one or more additional skin diseases or disorders in addition to acne vulgaris. In some embodiments, the subject has psoriasis, dermatitis, or a skin infection, an allergic reaction, hives, seborrhea, irritant contact dermatitis, allergic contact dermatitis, hidradenitis suppurative, allergic purpura. Pityriasis rosea, Dermatitis herpetiformis, erythema nodosum, erythema multiforme, lupus erythematosus, a bruise, actinic keratoses, keloid, lipoma, a sebaceous cyst, a skin tag, xanthelasma, basal cell carcinoma, squamous cell carcinoma, or Kaposi's sarcoma.

In some embodiments, the methods provided herein are used to characterize the outer surface of virtually any animal. In certain aspects, the methods are used to characterize the skin of a mammalian subject. For example, in some embodiments, the methods are used to characterize the skin of human, non-human primates, domesticated animals, such as livestock (e.g., cows, sheep, or pigs), dogs, cats, or rodents, such as mice, rats, or rabbits. In illustrative examples, the methods are used to analyze human skin.

### Exemplary Adhesive Tapes

In some embodiments, the adhesive tape is pliable. In some embodiments, the adhesive tape comprises a non-polar polymer adhesive. In some embodiments, the adhesive tape comprises a rubber-based adhesive.

In certain instances, non-polar, pliable adhesive tapes, including plastic-based adhesive tapes, are effective for obtaining epidermal samples from the skin. In certain instances, non-polar, pliable adhesive tapes, including plastic-based adhesive tapes, are more effective for obtaining epidermal samples from the skin than other types of adhesive tapes. Accordingly, in some embodiments, a non-polar, pliable adhesive tapes are applied in as few as 10 or less tape strippings, such as 9, 8, 7, 6, 5, 4, 3, 2, or 1 tape stripping, to obtain a sample. In some embodiments, the tape strippings method is employed to isolate a gene product from the epidermis of skin for gene expression analysis.

In some embodiments, the rubber based adhesive is a synthetic rubber-based adhesive. In some embodiments, the rubber based adhesive has high peel, high shear, and high tack. For example, in some embodiments, the rubber based adhesive has a peak force tack that is at least 25%, 50%, or 100% greater than the peak force tack of an acrylic-based tape such as D-squame™. D-squame.™ has been found to have a peak force of 2 Newtons. In some embodiments, the peak force of the rubber based adhesive used for methods provided herein is about 4 Newtons or greater. In some embodiments, the rubber based adhesive has adhesion that is greater than 2 times, 5 times, or 10 times that of acrylic based tape. D-squame™ has been found to have adhesion of 0.0006 Newton meters. In some embodiments, the rubber based tape provided herein has an adhesion of about 0.01 Newton meters using a texture analyzer. In some embodiments, the adhesive used in the methods provided herein has higher peel, shear and tack compared to other rubber adhesives, such as those used for medical application and Duct tape.

In some embodiments, the rubber-based adhesive is more hydrophobic than acrylic adhesives. In some embodiments, the rubber based adhesive is inert to biomolecules and to chemicals used to isolate biomolecules, including proteins and nucleic acids, such as DNA and RNA. In some embodiments, the rubber-based adhesive is relatively soft compared to other tapes such as D-squame™.

In some embodiments, the rubber-based adhesive is on a support, such as a film, that makes the tape pliable and flexible. In certain aspects, the tape is soft and pliable. As used herein, “pliable” tape is tape that is easily bent or shaped. As used herein, “soft and pliable” tape is tape that is easily bent or shaped and yields readily to pressure or weight. In some embodiments, the film is made of any of many possible polymers, provided that the tape is pliable and can be used with a rubber adhesive. In some embodiments, the film is a polyurethane film such as skin harvesting tape (Product No. 90068) available from Adhesives Research, Inc (Glen Rock, Pa.). In some embodiments, the thickness is varied provided that the tape remains pliable. For example, in some embodiments, the tape is about 0.5 mm to about 10 mm in thickness, such as about 1.0 to about 5.0 mm in thickness. In one example, the tape contains a rubber adhesive on a 3.0 mm polyurethane film.

### Isolation of Gene Products

In certain embodiments, the gene products are isolated from the epidermal samples. In some embodiments, the cells of the epidermal samples are lysed. In some embodiments, the cells of the epidermal samples are lysed and the gene products are isolated from lysed cells.

In certain embodiments, nucleic acid molecules are isolated from the lysed cells and cellular material by any number of means well known to those skilled in the art. For example, in some embodiments, any of a number of commercial products available for isolating nucleic acid molecules, including, but not limited to, RNeasy™ (Qiagen, Valencia, Calif.) and TriReagent™ (Molecular Research Center, Inc, Cincinnati, Ohio), is used. In some embodiments, the isolated nucleic acid molecules are then tested or assayed for particular nucleic acid sequences. In some embodiments, the isolated nucleic acid molecules are then tested or assayed for a nucleic acid sequence that represents a gene product of any of the genes listed in any of Tables 3, 6, 7, 8, or 9. Methods of detecting a target nucleic acid molecule within a nucleic acid sample are well known in the art. In some embodiments, detecting a target nucleic acid molecule involves a hybridization technique such as a microarray analysis or sequence specific nucleic acid amplification. In some embodiments, detecting a target nucleic acid molecule involves sequencing.

In some embodiments, one or more of the nucleic acid molecules in a sample provided herein, such as a as an epidermal sample, is amplified before or after they are isolated and/or detected. The term “amplified” refers to the process of making multiple copies of the nucleic acid from a single nucleic acid molecule. In some embodiments, the amplification of nucleic acid molecules is carried out in vitro by biochemical processes known to those of skill in the art. In some embodiments, the amplification agent is any compound or system that will function to accomplish the synthesis of primer extension products, including enzymes. It will be recognized that various amplification methodologies can be utilized to increase the copy number of a target nucleic acid in the nucleic acid samples obtained using the methods provided herein, before and after detection. Suitable enzymes for this purpose include, for example, E. coli DNA polymerase I, Taq polymerase, Klenow fragment of E. coli DNA polymerase I, T4 DNA polymerase, other available DNA polymerases, T4 or T7 RNA polymerase, polymerase muteins, reverse transcriptase, ligase, and other enzymes, including heat-stable enzymes (i.e., those enzymes that perform primer extension after being subjected to temperatures sufficiently elevated to cause denaturation or those using an RNA polymerase promoter to make a RNA from a DNA template, i.e. linearly amplified aRNA).

Suitable enzymes will facilitate incorporation of nucleotides in the proper manner to form the primer extension products that are complementary to each nucleotide strand. Generally, the synthesis will be initiated at the 3′-end of each primer and proceed in the 5′-direction along the template strand, until synthesis terminates, producing molecules of different lengths. There can be amplification agents, however, that initiate synthesis at the 5′-end and proceed in the other direction, using the same process as described above. In any event, the method provided herein is not to be limited to the amplification methods described herein since it will be understood that virtually any amplification method can be used.

In some embodiments, polymerase chain reaction (PCR) is employed for nucleic acid amplification (described, e.g., in U.S. Pat. Nos. 4,683,202 and 4,683,195). It will be understood that optimal conditions for a PCR reaction can be identified using known techniques. In one illustrative example, RNA is amplified using the MessageAmp™ aRNA kit (as disclosed in the Examples herein).

In some embodiments, the primers for use in amplifying the polynucleotides of the invention are prepared using any suitable method, such as conventional phosphotriester and phosphodiester methods or automated embodiments thereof so long as the primers are capable of hybridizing to the polynucleotides of interest. One method for synthesizing oligonucleotides on a modified solid support is described in U.S. Pat. No. 4,458,066. The exact length of primer will depend on many factors, including temperature, buffer, and nucleotide composition. The primer must prime the synthesis of extension products in the presence of the inducing agent for amplification.

Primers used according to the method of the invention are complementary to each strand of nucleotide sequence to be amplified. The term “complementary” means that the primers must hybridize with their respective strands under conditions, which allow the agent for polymerization to function. In other words, the primers that are complementary to the flanking sequences hybridize with the flanking sequences and permit amplification of the nucleotide sequence. The 3′ terminus of the primer that is extended can have perfect base paired complementarity with the complementary flanking strand, or can hybridize to the flanking sequences under high stringency conditions.

In some embodiments, upon isolation and optional amplification, expression of one or more genes is analyzed. Analyzing expression includes any qualitative or quantitative method for detecting expression of a gene, many of which are known in the art. Non-limiting methods for analyzing polynucleotides and polypeptides are discussed below. The methods of analyzing expression of the present invention can utilize a biochip, or other miniature high-throughput technology, for detecting expression of two or more genes.

In some embodiments, the methods provided involve isolation of RNA, including messenger RNA (mRNA), from a skin sample. In some embodiments, RNA is single stranded or double stranded. In some embodiments, enzymes and conditions optimal for reverse transcribing the template to DNA well known in the art are used. In some embodiments, the RNA is amplified to form amplified RNA. In some embodiments, the RNA is subjected to RNAse protection assays. In some embodiments, a DNA-RNA hybrid that contains one strand of each is used. In some embodiments, a mixture of polynucleotides is employed, or the polynucleotides produced in a previous amplification reaction, using the same or different primers are used. In certain examples, a nucleic acid to be analyzed is amplified after it is isolated. It is not necessary that the sequence to be amplified be present initially in a pure form; it may be a minor fraction of a complex mixture.

### Detection Methods

In some embodiments, a microarray is employed for detection of an expressed gene product. The manufacture and use of biochips such as those involving microarrays, also known as bioarrays, are known in the art. (For reviews of Biochips and microarrays see, e.g., Kallioniemi O. P., “Biochip technologies in cancer research,” Ann Med, March; 33(2):142 7 (2001); and Rudert F., “Genomics and proteomics tools for the clinic,” Curr Opin. Mol. Ther., December; 2(6):633 42 (2000)) Furthermore, a number of biochips for expression analysis are commercially available (See e.g., microarrays available from Sigma-Genosys (The Woodlands, Tex.); Affymetrix (Santa Clara, Calif.), and Full Moon Biosystems (Sunnyvale, Calif.)). In some embodiments, such microarrays are analyzed using blotting techniques similar to those discussed below for conventional techniques of detecting polynucleotides and polypeptides. In some embodiments, detailed protocols for hybridization conditions are available through manufacturers of microarrays. In some embodiments, a microarray provide for the detection and analysis of at least 10, 20, 25, 50, 100, 200, 250, 500, 750, 1000, 2500, 5000, 7500, 10,000, 12,500, 25,000, 50,0000, or 100,000 genes.

In some embodiments, for microarray expression analysis, approximately 0.1 to 1 milligram, typically 1 to 10 nanograms of RNA are isolated from an epidermal sample, for example, an epidermal sample obtained using a tape stripping method disclosed herein. In some embodiments, isolated RNA is then amplified. In some embodiments, the amplified RNA is then used for hybridization to sequence specific nucleic acid probes on a biochip. In some embodiments, amplification typically results in a total of at least 1 microgram, and more typically at least 20 micrograms of amplified nucleic acid. In some embodiments, amplification is performed using a commercially available kit, such as MessageAMp™ RNA kit (Ambion Inc.). In some embodiments, isolated RNA is labeled before contacting the biochip such that binding to the target array can be detected using streptavidin. In some embodiments, isolated RNA is labeled with a detectable moiety, such as, but not limited to, a fluorescent moiety, a dye, or a ligand, such as biotin. In some embodiments, the nucleic acid probes of the microarray bind specifically to one or more of the gene products of the genes listed in any of Tables 3, 6, 7, 8, or 9, or a complement thereof.

In some embodiments, hybridization of amplified nucleic acids to probes on a microarray is typically performed under stringent hybridization conditions. Conditions for hybridization reactions are well known in the art and are available from microarray suppliers. For example, in some embodiments, hybridization of a nucleic acid molecule with probes found on a microarray is performed under moderately stringent or highly stringent physiological conditions, as are known in the art. For example, in some embodiments, hybridization on a microarray is performed according to manufacturer's (Affymetrix) instructions. For example, in some embodiments, hybridization is performed for 16 hours at 45° C. in a hybridization buffer, such as 100 mM MES, 1 M [Na⁺], 20 mM EDTA, 0.01% Tween 20. In some embodiments, washes are performed in a low stringency buffer ((6×SSPE, 0.01% Tween 20) at 25° C. followed by a high stringency buffer (100 mM MES, 0.1M [Na⁺], 0.01% Tween 20) at 5° C. In some embodiments, washes are performed using progressively higher stringency conditions: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). In some embodiments, washing is carried out using only one of these conditions, for example, high stringency conditions. In some embodiments, washing is carried out using each of the conditions. In some embodiments, washing is carried out using each of the conditions, for 10 to 15 minutes each, in the order listed above, optionally repeating any or all of the steps listed.

In some embodiments, other microfluidic devices and methods for analyzing gene expression, including those in which more than one gene can be analyzed simultaneously and those involving high-throughput technologies, are used for the methods provided herein.

Quantitative measurement of expression levels using bioarrays is also known in the art, and typically involves a modified version of a traditional method for measuring expression as described herein. For example, such quantitation can be performed by measuring a phosphor image of a radioactive-labeled probe binding to a spot of a microarray, using a phospohor imager and imaging software.

Many statistical techniques are known in the art, which can be used to determine whether a statistically significant difference in expression is observed at a 90% or preferably a 95% confidence level.

In some embodiments, RNAse protection assays is used where RNA is the polynucleotide to be detected in the method. In this procedure, a labeled antisense RNA probe is hybridized to the complementary polynucleotide in the sample. The remaining unhybridized single-stranded probe is degraded by ribonuclease treatment. The hybridized, double stranded probe is protected from RNAse digestion. After an appropriate time, the products of the digestion reaction are collected and analyzed on a gel (see for example Ausubel et al., CURRENT PROTOCOLS IN MOLECULAR BIOLOGY, section 4.7.1 (1987)). As used herein, “RNA probe” refers to a ribonucleotide capable of hybridizing to RNA in a sample of interest. Those skilled in the art will be able to identify and modify the RNAse protection assay specific to the polynucleotide to be measured, for example, probe specificity can be altered, hybridization temperatures, quantity of nucleic acid etc. Additionally, a number of commercial kits are available, for example, RiboQuant™ Multi-Probe RNAse Protection Assay System (Pharmingen, Inc., San Diego, Calif.).

In another embodiment, a nucleic acid in the sample is analyzed by a blotting procedure, typically a Northern blot procedure. For blotting procedures polynucleotides are separated on a gel and then probed with a complementary polynucleotide to the sequence of interest. For example, RNA is separated on a gel transferred to nitrocellulose and probed with complementary DNA to one of the genes disclosed herein. In some embodiments, complementary probe is labeled such as radioactively or chemically.

In some embodiments, detection of a nucleic acid includes size fractionating the nucleic acid. Methods of size fractionating nucleic acids are well known to those of skill in the art, such as by gel electrophoresis, including polyacrylamide gel electrophoresis (PAGE). For example, in some embodiments, the gel is a denaturing 7 M or 8 M urea-polyacrylamide-formamide gel. In some embodiments, size fractionating the nucleic acid is accomplished by chromatographic methods known to those of skill in the art.

In some embodiments, the detection of nucleic acids is performed by using radioactively labeled probes. In some embodiments, any radioactive label is employed which provides an adequate signal. Other labels include ligands, colored dyes, and fluorescent molecules, which, in some embodiments, serve as a specific binding pair member for a labeled ligand, and the like. The labeled preparations are used to probe for a nucleic acid by the Southern or Northern hybridization techniques, for example. Nucleotides obtained from samples are transferred to filters that bind polynucleotides. After exposure to the labeled polynucleotide probe, which will hybridize to nucleotide fragments containing target nucleic acid sequences, the binding of the radioactive probe to target nucleic acid fragments is identified by autoradiography (see Genetic Engineering, 1 ed. Robert Williamson, Academic Press (1981), pp. 72 81). The particular hybridization technique is not essential to the performance of the method provided. Hybridization techniques are well known or easily ascertained by one of ordinary skill in the art. As improvements are made in hybridization techniques, they can readily be applied in the method of the invention.

In some embodiments, probes according for use in the methods provided selectively hybridize to a target gene. In some embodiments, the probes are spotted on a bioarray using methods known in the art. As used herein, the term “selective hybridization” or “selectively hybridize,” refers to hybridization under moderately stringent or highly stringent conditions such that a nucleotide sequence preferentially associates with a selected nucleotide sequence over unrelated nucleotide sequences to a large enough extent to be useful in detecting expression of a skin marker. It will be recognized that some amount of non-specific hybridization is unavoidable, but is acceptable provide that hybridization to a target nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar (i.e., homologous) nucleic acid molecule other than the target nucleic acid molecule.

In some embodiments, conditions that allow for selective hybridization are determined empirically, or estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual (Cold Spring Harbor Laboratory Press 1989)). An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42EC (moderate stringency conditions); and 0.1×SSC at about 68EC (high stringency conditions). In some embodiments, washing is carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

In some embodiments, a method for detecting one or more genes employs the detection of a polypeptide product of one of these genes. For example, in some embodiments, polypeptide products of one of the genes disclosed herein as associated with psoriasis or irritated skin, is analyzed. The levels of such gene products are indicative of acne vulgaris when compared to a normal or standard polypeptide profiles in a similar tissue. In this regard, the sample, as described herein, is used as a source to isolate polypeptides. For example, in some embodiments, following skin stripping, using the methods described above, cells isolated from the stratum corneum are lysed by any number of means, and polypeptides obtained from the cells. In some embodiments, these polypeptides are quantified using methods known to those of skill in the art, for example by protein microarrays, or ELISA analysis.

In another embodiment, provided are methods for obtaining gene expression data from amplified nucleic acids that compensates for variability in amplification reactions. In this method, relative expression of a target nucleic acid molecule and a control nucleic acid molecule is compared to obtain relevant expression data. Accordingly, in certain embodiments, a ΔCt value is determined in order to identify gene expression changes. In some embodiments, this value and method is used to identify differential gene expression in any tissue, including the tape stripped skin samples provided herein. Such method is especially useful, where it is relatively difficult to obtain sufficient RNA from a control sample.

The Cₜ value is the experimentally determined number of amplification (e.g. PCR) cycles required to achieve a threshold signal level (statistically significant increase in signal level (e.g. fluorescence) over background) for mRNAₓ and a control mRNA (Gibson, Heid et al. 1996; Heid, Stevens et al. 1996). The Ct values are typically determined using a target nucleic acid (e.g. mRNAx) primer and probe set, and a control mRNA primer and probe set. A ΔCₜ value is calculated by calculating a difference in the number of amplification cycles required to reach a threshold signal level between the target nucleic acid molecule and the control nucleic acid molecule. A difference in the ΔCₜ value at a target area versus another area of a subject's skin, such as a normal area, or an unaffected area, is indicative of a change in gene expression of the target nucleic acid molecule at the target area. Using this value, altered expression is detected by comparing expression of the target nucleic acid molecule with expression of a control nucleic acid molecule. The ΔCₜ value is useful for characterizing the physiologic state of the epidermis without reference to a calibration site. Such methods provide the advantage that it is not necessary to obtain a nucleic acid sample from a control site, where it may be difficult to obtain sufficient nucleic acid molecules.

Accordingly, provided herein is a method for detecting a change in gene expression, including: applying a first adhesive tape to a target area of skin and a second adhesive tape to an unaffected area of the skin, in a manner sufficient to isolate an epidermal sample adhering to the first adhesive tape and the second adhesive tape, wherein the epidermal samples comprise nucleic acid molecules; and for each of the target area sample and the normal area sample, amplifying a target nucleic acid molecule and a control nucleic acid molecule. For each of the target area sample and the normal area sample, a target nucleic acid molecule and a control nucleic acid molecule are amplified and identifying, and ΔCₜ value by calculating a difference in the number of amplification cycles required to reach a threshold signal level between the target nucleic acid molecule and a control nucleic acid molecule, wherein a difference in the ΔCₜ value at the target area versus the normal area is indicative of a change in gene expression of the target nucleic acid molecule at the target area. The Ct values are typically determined in the same amplification experiment (e.g. using separate reaction wells on the same multi-well reaction plate) using similar reaction conditions to other reactions.

In some embodiments, the method for detecting a change in gene expression is used along with the other embodiments provided herein to identify changes in gene expression. For example, In some embodiments, the method is used to diagnose acne vulgaris. In certain aspects, the method is used to detect a change in expression for any of the genes listed in Tables 3, 6, 7, 8, or 9, to assist in a characterization of a skin area as involving acne vulgaris.

### Application of the Methods

In some embodiments, provided herein are non-invasive methods for diagnosing acne vulgaris in a subject, including: applying an adhesive tape to a lesion suspected of being an acne lesion on the skin of the subject in a manner sufficient to isolate an epidermal sample adhering to the adhesive tape, wherein the epidermal sample includes a target gene product. The target gene product is then detected, wherein an altered expression of the target gene product as compared with expression in an epidermal sample from a sample not having acne vulgaris is indicative of acne vulgaris. In some embodiments, two or more target gene products are detected. In some embodiments, a target gene product is a nucleic acid molecule. In some embodiments, a target gene product is a polypeptide. In some embodiments, the target gene product is selected from among a gene product of any of Tables 3, 6, 7 or 8. In some embodiments, the target gene product is selected from among a gene product of Table 8. In some embodiments, the target gene product is selected from among a gene product of Table 9.

In some embodiments, provided herein are non-invasive methods for identifying a predictive skin marker for response to treatment for acne vulgaris, including: applying an adhesive tape to the skin of a subject afflicted with acne vulgaris at a first time point, in a manner sufficient to isolate an epidermal sample including gene products and treating the subject for acne vulgaris. In some embodiments, it is then determined whether the subject has responded to the treatment, and if so, whether expression of a gene product in the epidermal sample is predictive of response to treatment. In some embodiments, a target gene product is a nucleic acid molecule. In some embodiments, a target gene product is a polypeptide.

In certain embodiments, expression of a gene product in the epidermal sample is predictive of response to treatment if expression of the gene product at the first time point is different in subjects that respond to treatment compared to subjects that do not respond to treatment. It will be understood that a variety of statistical analysis can be performed to identify a statistically significant association between expression of the gene product and response of the subject to the treatment. In some embodiment, the expression of the gene product, in certain examples, is elevated in subjects that will not respond to treatment. Furthermore, expression of the gene product can predict a level of response to treatment, for example partial or temporary response to treatment versus a full response. In some embodiments, a target gene product is a nucleic acid molecule. In some embodiments, a target gene product is a polypeptide.

In some embodiments, provided herein is a non-invasive method for predicting response to treatment for acne vulgaris, including applying an adhesive tape to the skin of a subject afflicted with acne vulgaris in a manner sufficient to isolate an epidermal sample that includes a gene product. In some embodiments, a target gene product is detected in the epidermal sample, whose expression is indicative of a response to treatment, thereby predicting response to treatment for acne vulgaris. In some embodiments, a target gene product is a nucleic acid molecule. In some embodiments, a target gene product is a polypeptide.

In some embodiments, methods are provided herein for identifying a predictive skin biomarker for acne vulgaris, or predicting response to treatment by detecting a predictive skin biomarker in a subject having acne vulgaris. In some embodiments, the predictive skin biomarker is a target gene product detected using the methods provided herein. In some embodiments, a target gene product is a nucleic acid molecule. In some embodiments, a target gene product is a polypeptide. In some embodiments, the predictive skin biomarker is a biomarker for acne vulgaris. In some embodiments, the treatment for acne vulgaris is a topical treatment, phototherapy, a systemic medication, or a biologic.

Certain embodiments provided herein, are based in part on the discovery that the expression of certain genes can be used to monitor response to therapy. Accordingly, in another embodiment, provided herein is a method for monitoring a response of a human subject to treatment for acne vulgaris, including applying an adhesive tape to the skin of the subject being treated for the disease or condition at a first time point and at least a second time point, in a manner sufficient to isolate an epidermal sample adhering to the adhesive tape at the first time point and at the second time point. In some embodiments, the epidermal sample includes a gene product, wherein a change in expression of the gene product between the first time point and the second time point is indicative of a change in severity or level of acne vulgaris. In some embodiments, a target gene product is a nucleic acid molecule. In some embodiments, a target gene product is a polypeptide.

In some embodiments, provided herein is a method for detecting a response of a subject to treatment for acne vulgaris or monitoring the response of a subject to treatment for acne vulgaris over a period of time, comprising: treating the subject for a skin disease or condition state; applying an adhesive tape to the skin of the subject in a manner sufficient to isolate an epidermal sample, wherein the epidermal sample includes a gene product; and detecting a target gene product in the sample. Expression of the target gene product is informative regarding pathogenesis of acne vulgaris. Therefore, the method identifies a response of the subject to treatment for acne vulgaris. In some embodiments, a target gene product is a nucleic acid molecule. In some embodiments, a target gene product is a polypeptide.

In some embodiments, the treatment for acne vulgaris is selected from among an antibiotic, a retinoid, a hormone, or an aldosterone receptor antagonist. In some embodiments, the treatment for acne vulgaris is selected from among benzoyl peroxide, asapalene, azalaic acid, clindamycin, cephalexin, dapsone, dropirenone, doxycycline, erythromycin. ethinyl estradiol, isotretinoin, magnesium hydroxide, minocycline, salicylic acid, sodium sulfacetamide, sulfamethoxazole, spironolactone, tazarotene, tretinoin or trimethoprim. In some embodiments, the treatment is administered topically. In some embodiments, the treatment is administered orally. In some embodiments, the treatment is a combination of two or more agents for the treatment of acne vulgaris. In some embodiments, the treatment comprises benzoyl peroxide and an additional treatment for acne vulgaris. In some embodiments, the treatment comprises benzoyl peroxide and an antibiotic, a retinoid, a hormone, or an aldosterone receptor antagonist. In some embodiments, the treatment comprises benzoyl peroxide and asapalene, azalaic acid, clindamycin, cephalexin, dapsone, dropirenone, doxycycline, erythromycin. ethinyl estradiol, isotretinoin, magnesium hydroxide, minocycline, salicylic acid, sodium sulfacetamide, sulfamethoxazole, spironolactone, tazarotene, tretinoin or trimethoprim. In some embodiments, the treatment comprises benzoyl peroxide and adapalene. In some embodiments, the treatment comprises Epiduo® Gel (Galderma Laboratories, Ft. Worth, Tex.) (adapalene (0.1%) and benzoyl peroxide (2.5%) in a gel vehicle). In some embodiments, the treatment comprises benzoyl peroxide and clindamycin. In some embodiments, the treatment comprises Clindoxyl® Gel (Duac gel in U.S.) (1% clindamycin phosphate and 5% benzoyl peroxide in a gel vehicle).

In some embodiments, the detection of the gene product is a qualitative detection of whether the target gene product is expressed. In some embodiments, the detection of the target gene product is quantitative assessment of the expression level of the target gene product. In some embodiments, the method is performed both prior to treatment and after treatment. In some embodiments, the method is performed after treatment, but before a change in severity or level of acne vulgaris is observed visually. In some embodiments, the method is performed at multiple time point during treatment.

Time points for the monitoring and response-to-treatment methods provided herein, include any interval of time. In some embodiments, the time points are 1 day, 2 days, 3 days, 4 days, 5 days 6 days, 1 week, 2 weeks, 3, weeks, 4 weeks, 1 month, 2 months, 3 months, 4 months, 5 months, 6 months, 7 months, 8 months, 9 months, 10 months, 11 months, 1 year, 2 years or longer apart.

In some embodiments, skin samples are obtained at any number of time points, including 2, 3, 4, 5, 6, 7, 8, 9, 10, 11, 12, or more time points.

In some embodiments, comparison of expression analysis data from different time points is performed using any of the known statistical methods for comparing data points to assess differences in the data, including time-based statistical methods such as control charting. In some embodiments, the identity, severity or level of acne vulgaris is identified in the time series, for example, by comparing expression levels to a cut-off value, or by comparing changes in expression levels to determine whether they exceed a cut-off change value, such as a percent change cut-off value. In certain aspects, the first time point is prior to treatment, for example, prior to administration of a therapeutic agent, and the second time point is after treatment.

In some embodiments, the change in expression levels of at least one gene product is an increase or decrease in expression. Depending on the target gene product, an increase or decrease indicates a response to treatment, or a lack of response. For example, in some embodiments, the gene product is a nucleic acid that encodes a protein such as a protein expressed by a gene of any of Tables 3, 6, or 8, and a decrease in expression at the second time point as compared to the first time point is indicative of positive response to treatment for acne vulgaris. As another example, in some embodiments, the gene product detected is a polypeptide that is expressed by a gene of any of Tables 3, 6, or 8 and a decrease in expression at the second time point as compared to the first time point is indicative of positive response to treatment for acne vulgaris. In some embodiments, the gene product is a nucleic acid that encodes a protein such as a protein expressed by a gene of any of Tables 3, 7, or 8, and an increase in expression at the second time point as compared to the first time point is indicative of positive response to treatment for acne vulgaris. As another example, in some embodiments, the gene product detected is a polypeptide that is expressed by a gene of any of Tables 3, 7, or 8 and an increase in expression at the second time point as compared to the first time point is indicative of positive response to treatment for acne vulgaris.

In some embodiments, more than one target gene product is detected. In some embodiments, a population of target gene products are detected. In some embodiments, the method for detecting a population of target gene products is performed using a microarray.

In some embodiments, provided herein are methods for characterizing skin of a subject, including applying an adhesive tape to a target area of skin in a manner sufficient to isolate an epidermal sample adhering to the adhesive tape, wherein the epidermal sample includes a gene product. In some embodiments, a gene product whose expression is informative of a skin disease or pathological skin state is then detected in the epidermal sample. For example, in some embodiments, the expression of a gene product of a gene listed in any of Tables 3, 6, 7 or 8 is detected in the epidermal sample to characterize the subject as having acne vulgaris. In some embodiments, the level expression of a gene product of a gene listed in any of Tables 3, 6, 7 or 8 is detected in the epidermal sample to characterize the subject as having a particular level of severity of acne vulgaris. In some embodiments, the level expression of a gene product of a gene listed in any of Tables 3, 6, 7 or 8 is detected in the epidermal sample to characterize the subject as sensitive to or having an increased risk of developing acne vulgaris. In some embodiments, the level expression of a gene product of a gene listed in any of Tables 3, 6, 7 or 8 is detected in the epidermal sample to characterize the subject as sensitive to or having an increased risk of developing acne vulgaris. In some embodiments, the level expression of a gene product of a gene listed in any of Tables 3, 6, 7 or 8 is detected in the epidermal sample to characterize the subject as a candidate for a particular treatment for acne vulgaris. In some embodiments, the level expression of a gene product of a gene listed in any of Tables 3, 6, 7 or 8 is detected in the epidermal sample to characterize the subject as sensitive to a particular treatment for acne vulgaris.

In a certain embodiments, the effects of an agent, such as a test agent, on the skin are determined. In some embodiments, cells of the skin, such as epidermal cells, including keratinocytes and melanocytes, or dermal cells, such as fibroblasts, are contacted with a test agent. The expression of biomarkers for acne vulgaris is then detected. In some embodiments, the methods comprise: contacting a target area of the skin with the agent and applying an adhesive tape to the target area of the skin in a manner sufficient to isolate an epidermal sample adhering to the adhesive tape, wherein the epidermal sample includes a gene product. In some embodiments, the gene product is isolated from the epidermal sample to determine an expression profile for the target site of the skin. In some embodiments, the expression profile is indicative of a state of the skin, thereby providing a determination of the effect of the agent on the skin. The expression profile can be obtained using a microarray, as discussed in more detail herein. In some embodiments, the gene product is an nucleic acid molecule or a polypeptide.

In some embodiments, provided herein are methods for screening test agents for the treatment of acne vulgaris. In a certain embodiments, provided herein are methods for screening agents or identifying agents that cause acne vulgaris or that increase the risk of developing acne vulgaris.

In some embodiments, the agent is applied until or before any visual effects of application of the agent become evident. In some embodiments, the agent is applied for between 1 second to 12 hours to a skin site, such as between about 0.5 and 2 hours before it is removed and tape stripping is performed on the skin site contacted with the agent. The conditions under which contact is made are variable and will depend upon the type of agent, the type and amount of cells in the skin to be tested, the concentration of the agent in the sample to be tested, as well as the time of exposure to the agent. It will be understood that routine experimentation can be used to optimize conditions for contacting skin with the agent.

As illustrated in the examples, expression of about 806 genes was altered in inflammatory acne lesions versus uninvolved skin. In some embodiments, changes of skin state from normal to an inflammatory acne lesion, are accompanied by changes in at least or about 806 genes. In some embodiments, methods provided herein characterize skin by analyzing expression of 2 or more, 5 or more, 10 or more, 25 or more, 50 or more, 100 or more, 500 or more, or all of the genes listed in Tables 3. In certain examples, expression is detected for a gene listed in Tables 3, 6, 7 or 8, which lists genes identified in the studies disclosed herein with the most dramatic expression changes in inflammatory acne lesions. In some embodiments, a detected gene product is an expression product of a gene listed in Tables 3, 6, 7 or 8. In some embodiments, a detected gene product is an expression product of a gene listed in Tables 3, 7 or 8, wherein a down-regulation of the nucleic acid in a tape stripped skin is indicative of an inflammatory acne lesion. In some embodiments, a detected gene product is an expression product of a gene listed in Tables 3, 6 or 8, wherein an upregulation of the nucleic acid in a tape stripped skin is indicative of an inflammatory acne lesion.

In some embodiments where expression of more than 1 gene is analyzed, the detection is performed using a microarray. In some examples, the microarray includes an array of sequence specific nucleic acid probes. In some embodiments, the microarray includes an array of sequence specific nucleic acid probes directed to 2 or more, 10 or more, 25 or more, 50 or more, 100 or more, 500 or more, 1000 or more, or all of the genes listed in Table 3, or the subset of genes listed in Table 6, or the subset of genes listed in Table 7, or the subset of genes listed in Table 8, or the subset of genes listed in Table 9.

In some embodiments, provided herein is a microarray that includes an array of probes. In some embodiments, the microarray includes an array of probes directed to 2 or more, 10 or more, 25 or more, 50 or more, 100 or more, 500 or more, 1000 or more, or all of the genes listed in Table 3, or the subset of genes listed in Table 6, or the subset of genes listed in Table 7, or the subset of genes listed in Table 8, or the subset of genes listed in Table 9.

In some embodiments, a method for identifying an expression profile indicative of acne vulgaris in a subject comprises applying an adhesive tape to an area of skin suspect of being an acne lesion in a manner sufficient to isolate an epidermal sample adhering to the adhesive tape, wherein the epidermal sample includes gene products, and applying the gene products to a microarray. In some embodiments, the gene products are nucleic acid molecules or polypeptides. In some embodiments, the gene products are isolated from the epidermal sample before being applied to the microarray. For example, in some embodiments, the nucleic acid molecules or polypeptides are isolated from the epidermal sample before being applied to the microarray. In some embodiments, relative expression levels of at least 10 genes is then determined using the microarray; wherein an altered relative expression level for at least 2, 3, 4, 5, 6, 7, 8, 9, or each of the at least 10 genes as compared with expression in an epidermal sample from a normal or uninvolved skin sample identifies the subject as having acne vulgaris, thereby identifying the expression profile indicative of acne vulgaris. In some embodiments, the nucleic acid molecules are RNA molecules.

In some embodiments, the relative amount of the gene product is increased in an epidermal skin sample from an acne lesion or an epidermal skin sample from a suspected acne lesion compared to a control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the relative amount of the gene product is decreased in an epidermal skin sample from an acne lesion or an epidermal skin sample from a suspected acne lesion compared to a control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the control is a normal skin sample. In some embodiments, the control is a value obtained from a database of relative expression values. In some embodiments, the control is a value obtained from a known relative expression values.

In some embodiments, a greater than 2-fold, 3-fold, 4-fold, 5-fold, 6-fold, 7-fold, 8-fold, 9-fold, 10-fold increase or decrease in expression of a gene product is used as a cut-off value for identifying an acne vulgaris skin marker. In some embodiments, a greater than about 4-fold increase or decrease in expression of a gene product is used as a cut-off value for identifying an acne vulgaris skin marker. The Examples provided herein illustrate the identification of acne vulgaris skin markers. In certain examples, there is at least a 2-fold, 3-fold, 4-fold, 5-fold, 6-fold, 7-fold, 8-fold, 9-fold, 10-fold difference in levels between a skin sample from an acne lesion and non-lesional skin. In certain examples, there is at least 4-fold difference in levels between a skin sample from an acne lesion and non-lesional skin. In certain examples, there is at least a 2-fold, 3-fold, 4-fold, 5-fold, 6-fold, 7-fold, 8-fold, 9-fold, 10-fold difference in levels between a skin sample from an acne lesion and a skin sample from an acne lesion following administration of a treatment for acne vulgaris. In certain examples, there is at least 4-fold difference in levels between a skin sample from an acne lesion and a skin sample from an acne lesion following administration of a treatment for acne vulgaris. In some embodiments, the skin sample from an acne lesion following administration of a treatment for acne vulgaris is obtained 1, 2, 3, 4, 5, 6, 7, 8, 9, 10, 11, 12 weeks or longer following treatment. In some embodiments, the skin sample from an acne lesion following administration of a treatment for acne vulgaris is obtained 2 weeks following treatment. In some embodiments, the skin sample from an acne lesion following administration of a treatment for acne vulgaris is obtained 4 weeks following treatment. In some embodiments, the skin sample from an acne lesion following administration of a treatment for acne vulgaris is obtained 8 weeks following treatment. Exemplary acne vulgaris skin markers identified herein include a gene of Table 8. Exemplary acne vulgaris skin markers identified herein include a gene of Table 9.

In some embodiments, expression of a target gene believed to be involved in acne vulgaris is detected in an acne lesion using a tape stripping method provided herein. In some embodiments, if expression or elevated expression is detected, a treatment is administered to the subject that blocks a function of the target gene. Accordingly, in some embodiments, the methods provided herein are used to determine whether the subject is likely to respond to treatment with a biologic that targets a particular gene that exhibits elevated expression in an acne lesion.

As illustrated herein, acne lesions express increased levels of genes listed in Table 6. Accordingly, in some embodiments, methods herein to characterize an acne lesion are used to confirm that acne lesions are expressing a gene listed in Table 6 before a subject is treated for acne vulgaris. As illustrated herein, acne lesions express decreased levels of genes listed in Table 7. Accordingly, in some embodiments, methods herein to characterize an acne lesion are used to confirm that acne lesions are expressing a decrease level of a gene listed in Table 7 before a subject is treated for acne vulgaris.

In another embodiment, provided herein is a method wherein tape stripping is used to tape harvest skin sites in need of classification. In some embodiments, epidermal samples are mailed to a laboratory of a service provider for development of an RNA profile which would indicate a classification (e.g. diagnosis of acne vulgaris) with greater than 95% confidence. In some embodiments, the RNA profile from the sample available over an intranet or internet for viewing by a customer of the service provider. In certain embodiments, a database is provided, of RNA profiles generated from epidermal samples.

In another embodiment, provided herein are kits are include one or more reagents or devices for the performance of the methods disclosed herein. In some embodiments, provided is a kit for isolation and detection of a nucleic acid from an epidermal sample, such as an epidermal sample from an acne lesion or a target area of skin suspected of being an acne lesion.

In some embodiments, the kit includes an adhesive tape for performing methods provided herein. In some embodiments, the kit includes an adhesive tape for tape stripping skin, such as rubber-based, pliable adhesive tape. Accordingly, in some embodiments, provided herein is a kit, including a pliable adhesive tape made up at least in part, of a non-polar polymer. In certain aspects, the tape includes a rubber adhesive. In an illustrative example, the tape can be skin harvesting tape available (Product No. 90068) from Adhesives Research, Inc (Glen Rock, Pa.). In some embodiments, the kit includes instructions for performing tape strippings or for analyzing gene expression.

In some embodiments, the kit includes nucleic acid or polypeptide isolation reagents.

In some embodiments, the kit includes one or more detection reagents, for example probes and/or primers for amplification of, or hybridization to, a target nucleic acid sequence whose expression is related to acne vulgaris. In some embodiments, the kit includes primers and probes for control genes, such as housekeeping genes. In some embodiments, the primers and probes for control genes are used, for example, in ΔCₜ calculations. In some embodiments, the probes or primers are labeled with an enzymatic, florescent, or radionuclide label. In some embodiments, the probe binds to a target nucleic acid molecule encoding a protein. In some embodiments, the probe is an antibody or ligand that binds the encoded protein. In some embodiments, probes are spotted on a microarray. In some embodiments, the microarray is provided in the kit.

The term “detectably labeled deoxyribonucleotide” refers to a deoxyribonucleotide that is associated with a detectable label for detecting the deoxyribonucleotide. For example, the detectable label may be a radiolabeled nucleotide or a small molecule covalently bound to the nucleotide where the small molecule is recognized by a well-characterized large molecule. Examples of these small molecules are biotin, which is bound by avidin, and thyroxin, which is bound by anti-thyroxin antibody. Other labels are known to those of ordinary skill in the art, including enzymatic, fluorescent compounds, chemiluminescent compounds, phosphorescent compounds, and bioluminescent compounds.

In some embodiments, the kit includes one or more primer pairs, including a forward primer that selectively binds upstream of a gene whose expression is associated with psoriasis or irritant dermatitis, for example, on one strand, and a reverse primer, that selectively binds upstream of a gene involved in psoriasis or irritant dermatitis on a complementary strand. Primer pairs according to this aspect of the invention are typically useful for amplifying a polynucleotide that corresponds to a skin marker gene associated with acne vulgaris using amplification methods described herein.

In some embodiments, a kit provided herein includes a carrier means being compartmentalized to receive in close confinement one or more containers such as vials, tubes, and the like, each of the containers comprising one of the separate elements to be used in a method provided herein. In some embodiments, a second container includes, for example, a lysis buffer. In some embodiments, the kit includes a computer-type chip on which the lysis of the cell will be achieved by means of an electric current.

### EXAMPLES

These examples are provided for illustrative purposes only and not to limit the scope of the claims provided herein.

### Example 1

### Objectives

A feasibility study EGIR-01 previously demonstrated that it non-invasive cell harvesting technique, tape stripping, can be used to collect skin cells of the stratum corneum overlaying acne lesions and assess gene expression profiles within these cells. The current study EGIR-02 was designed to assess gene expression profiles in acne lesions before, during and after treatment with Clindoxyl and Epiduo.

The primary objectives were to assess if the change in gene expression profiles over time during treatment were predictive of clinical outcome with respect to efficacy and safety and also to assess at which time the earliest prediction of clinical outcome can be made. Secondary/exploratory goals were to understand the acne pathology and mechanism of actions of treatment by following the gene expression profiles of both inflammatory and non-inflammatory lesions over treatment duration.

Study Design and Duration

The study was single-blinded, randomized, comparative and split-face study in two clinical sites. Patients were tape-stripped in inflammatory acne lesions (IN), non-inflammatory lesions (NIN) and normal heath skin at the following time points: baseline, week 1, 2, 5 and 8.

Inclusion Criteria

In order to be considered for study enrollment, the subject were required to fulfill all of the following conditions or characteristics:

1. Capable of understanding and willing to provide signed and dated written voluntary informed consent (and any local or national authorization requirements) before any protocol specific procedures are performed.

2. Male or female subjects who are at least 16 years of age at time of consent.

3. Have mild-to-moderate acne vulgaris

4. Able to complete the study and to comply with study instructions.

Exclusion Criteria

The subjects with any of the following conditions or characteristics were excluded from study enrollment:

1. Has other generalized skin disorders not related to acne vulgaris, such as psoriasis, photosensitivity disorders, or eczema.

2. History of known or suspected intolerance to any of the ingredients of the EGIR tape or latex rubber.

3. Has used a topical product within 24 hours of study entry.

4. Subject with other abnormal clinical findings which the investigator feels may put the Subject at undue risk or may interfere with the study results.

5. Employees of investigator or Stiefel Laboratories, or an immediate family member (partner, offspring, parents, siblings or sibling's offspring) of an employee.

Test Product, Dose and Mode of Administration

All enrolled subjects were tape-stripped on 6 separate sites; 3 on each side of the face, i.e. on each half receiving a separate 2 facial inflammatory acne lesions, one on each side of the face; 2 comedonal facial lesions, including normal appearing peri-comedonal skin, one on each side of the face; and 2 non-lesional facial control site, one on each side of the face.

The tape strip sample collection was performed by the principal investigator, or trained individuals delegated by the principal investigator, to obtain the superficial skin cells (stratum corneum). Tape stripping was performed at baseline, week 1, 2, 5 and week 8 visits, after the principal investigator confirmed eligibility and the Informed Consent Form was signed.

TABLE 1
STUDY FLOWCHART

| | | | | | Week 8 |
|---|---|---|---|---|---|
| | | | | | or Early |
| | Baseline | Week 1 | Week 2 | Week 5 | Withdrawal |
| | Day 1 | +/−3 days | +/−3 days | +/−3 days | +/−3 days |
| | Visit 1 | Visit 2 | Visit 3 | Visit 4 | Visit 5 |
| Informed Consent | ✓ | | | | |
| Inclusion Exclusion Criteria | ✓ | | | | |
| Demographics and Medical History Review | ✓ | | | | |
| of Systems | | | | | |
| UPT (Females of Child Bearing Potential) | ✓ | | ✓ | | ✓ |
| Investigator Static Global Assessment (ISGA) | ✓ | ✓ | ✓ | | ✓ |
| Lesions Counting | ✓ | ✓ | ✓ | | ✓ |
| Tolerability Assessments (Investigator) | | ✓ | ✓ | | ✓ |
| (erythema, dryness, peeling, irritant allergic | | | | | |
| contact dermatitis) | | | | | |
| Subjects' Global Change Assessment | | ✓ | ✓ | | ✓ |
| Facial Photography | ✓ | ✓ | ✓ | | ✓ |
| (front view, 2x lateral views) | | | | | |
| Concomitant Medication | ✓ | ✓ | ✓ | | ✓ |
| Adverse Events | | ✓ | ✓ | | ✓ |
| Dispense Study Product | ✓ | ✓ | ✓ | ✓ | |
| | Clindoxyl | Clindoxyl | Clindoxyl | Clindoxyl | |
| | Epiduo | Epiduo | only | only | |
| Return Study Product | | ✓ | ✓ | | ✓ |
| Diary Card Dispensed | ✓ | ✓ | | | |
| Diary Card Collection | | ✓ | ✓ | | |
| SKINDEX-29 (QcLI) | ✓ | | ✓ | | ✓ |
| Product Acceptability and Preference | | ✓ | ✓ | | ✓ |

Patients and Clinical Protocols

The study protocols were reviewed and approved by the Institutional Review Board and all subjects signed informed consent. Study subjects gave written informed consent prior to participation and the study was conducted according to the Declaration of Helsinki principles. All study subjects were at least 18 years of age, in good general health with documented diagnosis of facial acne vulgaris. Study exclusion criteria included application of topical medications to the lesion or use of systemic steroids within 30 days of tape stripping; presence of a generalized skin disorder, such as psoriasis, a photosensitivity disorder, or eczema; known allergy to tape or latex; use of sunscreen or topical moisturizer within 24 hours of tape stripping; and lesions with clinically overt bleeding, ulceration, or serous exudation. After informed consent, the suspicious pigmented lesion was taped stripped. As a control, each subject's normal appearing skin was also sampled by tape stripping.

Materials and Reagents

The EGIR tape kit contains 4 small circular adhesive discs, each 17 mm in diameter, with a polyurethane backing. The tape was purchased from Adhesives Research (Glen Rock, Pa.) and fabricated into discs with a polyurethane backing by Diagnostic Laminations Engineering (Oceanside, Calif.). Universal human reference RNA was purchased from Stratagene (San Diego, Calif.). Reverse transcriptase, TaqMan Universal Master Mix, which included all buffers and enzymes necessary for the amplification and fluorescent detection of beta-actin cDNA, were purchased from Applied Biosystems (Foster City, Calif.). MELT total nucleic acid isolation system was purchased from Ambion (Austin, Tex.). GeneChip® human genome U133 plus 2.0 arrays were purchased from Affymetrix (Santa Clara, Calif.). The GeneChip® human genome U133 plus 2.0 array comprises all of the probes if the GeneChip® Human Genome U133A 2.0 Array, which is a single array representing 14,500 well-characterized human genes that can be used to explore human biology and disease processes. The GeneChip® Human Genome U133A 2.0 Array represents 18,400 transcripts and variants, including 14,500 well-characterized human genes and is comprised of more than 22,000 probe sets and 500,000 distinct oligonucleotide features. The sequences from which these probe sets were derived were selected from GenBank®, dbEST, and RefSeq. The sequence clusters were created from the UniGene database (Build 133, Apr. 20, 2001) and then refined by analysis and comparison with a number of other publicly available databases, including the Washington University EST trace repository and the University of California, Santa Cruz Golden-Path human genome database (April 2001 release). The GeneChip® human genome U133 plus 2.0 array additionally contains 9,921 new probe sets representing approximately 6,500 new genes. These gene sequences were selected from GenBank, dbEST, and RefSeq. Sequence clusters were created from the UniGene database (Build 159, Jan. 25, 2003) and refined by analysis and comparison with a number of other publicly available databases, including the Washington University EST trace repository and the NCBI human genome assembly (Build 31).

RNA Isolation and Quantification

All tape strips were processed in the laboratory at DermTech International (La Jolla, Calif.). The RNA was extracted from tapes by means of MELT and quantified by TaqMan qPCR for beta-actin mRNA expression level, as per Wong et al (2004). RNA quality was assessed by microfluidic electrophoretic analysis using an Experion Automated Electrophoresis Station (BioRad, Inc., Hercules, Calif.).

RNA Amplification and Array Hybridization

RNA harvested from the EGIR tape strips was amplified using the Ovation FFPE RNA Amplification System (NuGEN Technologies, Inc., San Carlos, Calif.) and hybridized with Affymetrix human genome U133 plus 2.0 GeneChip, according to standard manufacturer protocols.

Gene Expression Analysis

The image files from scanning the Affymetrix GeneChips with the Affymetrix series 3000 scanner were converted to CEL-format files using the Affymetrix GeneChip Operating Software version 1.4 (GCOS v1.4). Normalization of GeneChip CEL files was carried out using the GCRMA software from Bioconductor (www.bioconductor.org). After filtering out for background and low expressed genes (level <100 for a gene target across all samples), data were imported into GeneSpring (Agilent, Santa Clara, Calif.). A supervised analysis was performed to identify genes differentially expressed between acne lesions and normal skin controls at Day 1 of visit. This was performed by ANOVA with multiple testing correction using the Westfall and Young permutation method (p<0.001, false discovery rate q<0.05). Cluster analysis was performed according to Eisen et al. Data were first log 2 transformed and then median centered for genes and samples. The resulting normalized data were further analyzed by the self organizing map algorithm and then clustered with Spearman rank correlation similarity metrics.

Gene ontology was performed with FuncAssociate 2.0 algorithm (llama.mshri.on.ca/funcassociate) and pathway analysis was analyzed by Ingenuity Pathways Analysis (IPA) system software version 8.5 (Ingenuity Systems, Inc., Redwood City, Calif.). Genes, with their corresponding identifiers and fold change values were uploaded for interrogation. After analysis, significance of the biological functions and the canonical pathways were tested by the Fisher's Exact test p-value to determine the probability that each biological/canonical pathways assigned to the data set is due to chance alone.

TABLE 2
Synopsis of EGIR-02 Study

| | |
|---|---|
| TITLE | EGIR-02 |
| | A Proof-of-Concept Study Towards Assessing Early Biomarkers in Acne |
| | Vulgaris |
| STUDY RATIONALE | The feasibility study EGIR-01 demonstrated that it is possible to use a non- |
| | invasive cell harvesting technique: tape stripping, to collect superficial cells |
| | (stratum corneum) overlaying acne lesions and assess gene expression profiles |
| | within these cells. |
| | This study is designed to assess gene expression profiles in acne lesions before, |
| | during and after treatment with Clindoxyl and Epiduo. The ultimate goal is to |
| | determine if the early change in expression of biomarkers for acne can be |
| | predictive of clinical safety and efficacy. Secondary goals are to understand |
| | mechanism of action of treatments and the pathophysiology of acne. |
| CLINICAL PHASE | 4 |
| INDICATION | Acne Vulgaris |
| OBJECTIVE(S) | Primary |
| | The primary objective of this amendment is to assess if the change in gene |
| | expression profiles over time during treatment is predictive of clinical outcome |
| | with respect to efficacy and safety, and to assess at which time the earliest |
| | prediction of clinical outcome can be made. |
| | Secondary/exploratory |
| | Increase understanding of acne pathology and mechanism of action of treatment |
| | by following the gene expression profiles of both inflammatory and non- |
| | inflammatory lesions over treatment duration. |
| STUDY DESIGN | A single-center, single-blind, randomized, comparative, split-face study. |
| STUDY DURATION | Visits will occur at the following time points: baseline, week 1, 2, 5 and week 8. |
| | At these visits skin samples will be harvested (tape stripped). |
| APPROXIMATE NUMBER OF | 45 |
| SUBJECTS | |
| NUMBER OF STUDY | 1 study site |
| CENTERS | |
| INCLUSION CRITERIA | The subject must fulfill all of the following conditions or characteristics in order |
| | to be considered for study enrollment: |
| | 1. Capable of understanding and willing to provide signed and dated written |
| | voluntary informed consent (and any local or national authorization |
| | requirements) before any protocol specific procedures are performed. |
| | 2. Male or female subjects who are at least 16 years of age at time of consent. |
| | 3. Have mild-to-moderate acne vulgaris. |
| | 4. Able to complete the study and to comply with study instructions. |
| EXCLUSION CRITERIA | The subjects with any of the following conditions or characteristics will be |
| | excluded from study enrollment: |
| | 1. Has other generalized skin disorders not related to acne vulgaris, such as |
| | psoriasis, photosensitivity disorders, or eczema. |
| | 2. History of known or suspected intolerance to any of the ingredients of the |
| | EGIR tape or latex rubber. |
| | 3. Has used a topical product within 24 hours of study entry. |
| | 4. Subject with other abnormal clinical findings which the investigator feels |
| | may put the Subject at undue risk or may interfere with the study results. |
| | 5. Employees of investigator or Stiefel Laboratories, or an immediate family |
| | member (partner, offspring, parents, siblings or sibling's offspring) of an |
| | employee. |
| CONCOMITANT | As C0000-404 |
| TREATMENT | |
| TEST PRODUCT, DOSE, AND | All enrolled subjects will be tape-stripped on 6 separate sites; 3 on each side of |
| MODE OF | the face, ie on each half receiving a separate 2 facial inflammatory acne lesions, |
| ADMINISTRATION | one on each side of the face; 2 comedonal facial lesion including normal |
| | appearing peri-comedonal skin, one on each side of the face; and 2 non-lesional |
| | facial control site, one on each side of the face. |
| | The tape strip sample collection will be performed by the principal investigator, |
| | or trained individuals delegated by the principal investigator, to obtain the |
| | superficial skin cells (stratum corneum). Tape stripping will be performed at |
| | baseline, week 1, 2, 5 and week 8 visits, after the principal investigator has |
| | confirmed eligibility and Informed Consent Form has been signed. |
| | The tape strip samples will be sent to DermTech International (DTI) for total |
| | RNA purification, and Affymetrix gene array analysis. |
| EFFICACY EVALUATION | The quality and purity of the extracted RNA will be assessed. |
| | Total RNA of selected subjects (post un-blinding), collected at baseline and |
| | week 8, will be used for compete gene expression profiling using Affymetrix |
| | gene chips. This will allow the identification of 10 of the most predictive |
| | biomarkers with respect to efficacy. The total RNA of the remaining samples |
| | will be analyzed using quantitative PCR for these 10 specific biomarkers. |
| PHARMACOKINETICS/ | The changes of gene expression will be monitored over the duration of |
| PHARMACODYNAMICS | treatment. To reduce cost, only selected samples (obtained at baseline vs. week |
| EVALUATION | 8) will be used for full gene chip analysis after un-blinding, to identify the 10 |
| | most appropriate/predictive biomarkers. Once the most compelling biomarkers |
| | are identified, the rest of the tape strips will be analyzed using PCR and a subset |
| | of selected biomarkers. |
| SAFETY EVALUATION | The quality and purity of the extracted RNA will be assessed. |
| | Total RNA of selected subjects (post un-blinding), collected at baseline and |
| | week 8, will be used for compete gene expression profiling using Affymetrix |
| | gene chips. This will allow the identification of 10 of the most predictive |
| | biomarkers with respect to safety. The total RNA of the remaining samples will |
| | be analyzed using quantitative PCR for these 10 specific biomarkers. |
| | For EGIR-02 part of the study: At every scheduled visit, post tape stripping, the |
| | subject will be assessed and interviewed by the principal investigator to inquire |
| | if any adverse events (AEs) or serious adverse events (SAEs) have occurred in |
| | relation to the tape stripping. If AEs have occurred they will be documented, |
| | assessed, and followed up on. If a subject has a related AE/SAE, the principal |
| | investigator will follow up on a weekly basis, until resolution or stabilization of |
| | the event. |
| STATISTICAL ANALYSIS | Primary Efficacy |
| | Total RNA will be used for gene expression profiling using Affymetrix gene |
| | chips. Changes in gene expression profiles of various lesions will be compared |
| | to baseline expression profiles and correlated to treatment arm. Finally, the most |
| | predictive biomarkers for clinical efficacy will be determined. Microarray data |
| | will be processed by GCRMA and quality of microarray data will be evaluated |
| | using simpleaffy script from Bioconductor. Differentially expressed genes |
| | between baseline and treatment profiles will be identified using an unpaired t- |
| | test with multiple testing correction ((p < 0.05, FDR < 0.05). The most predictive |
| | biomarkers for clinical efficacy will be identified using class prediction |
| | algorithms either “Prediction Analysis for Microarrays” (PAM), Random Forest |
| | or support vector machine (SVM). |
| | Secondary Efficacy |
| | Total RNA will be used for gene expression profiling using Affymetrix gene |
| | chips. Changes in gene expression profiles over time will be compared and |
| | correlated to the site of harvest or lesion type (inflammatory vs. non- |
| | inflammatory vs. non-lesional). Finally, the most predictive biomarkers for |
| | clinical efficacy will be determined. Microarray data will be processed by |
| | GCRMA and quality of microarray data will be evaluated using simpleaffy |
| | script from Bioconductor. Differentially expressed genes among inflammatory, |
| | non-inflammatory profiles and non-lesional skin profiles will be identified by |
| | statistical analysis of variance (ANOVA) with multiple testing correction ((p < |
| | 0.05, FDR < 0.05). The most predictive biomarkers for clinical efficacy will be |
| | identified using class prediction algorithms either “Prediction Analysis for |
| | Microarrays” (PAM), Random Forest or support vector machine (SVM). |
| ETHICAL CONSIDERATIONS | This study will be conducted in accordance with applicable laws and regulations |
| | and according to the Declaration of Helsinki (1996). |

The topical treatments administered in this study included Epiduo′ Gel (Galderma Laboratories, Ft. Worth, Tex.) and Clindoxyl® Gel (Stiefel). Epiduo® Gel contains adapalene (0.1%) and benzoyl peroxide (2.5%) in a gel vehicle. Clindoxyl® Gel (Duac gel in U.S.) is a combination of 1% clindamycin phosphate and 5% benzoyl peroxide in a gel vehicle.

Benzoyl peroxide for acne treatment is typically applied to the affected areas in gel or cream form, in concentrations of 2.5% increasing through the usually effective 5% to up to 10%. Research suggests that 5 and 10% concentrations are not significantly more effective than 2.5% and 2.5% is usually better tolerated. It commonly causes initial dryness and sometimes irritation, although the skin develops tolerance after a week or so. A small percentage of people are much more sensitive to it and liable to suffer burning, itching, peeling and possibly swelling. It is sensible to apply the lowest concentration and build up as appropriate. Once tolerance is achieved, increasing the quantity or concentration a second time and gaining tolerance at a higher level usually gives better subsequent acne clearance. Benzoyl peroxide works as a peeling agent, increasing skin turnover and clearing pores, thus reducing the bacterial count there as well as directly as an antimicrobial.

Results

The results of the gene expression microarray data are presented in Tables 3-9. The microarray data is presented as normalized relative fluorescence units (RFU). Gene symbols and additional information relating to the probesets contained on the GeneChip® human genome U133 plus 2.0 can be obtained from www.affymetrix.com. Gene symbols and Genebank accession numbers associated with the various probesets of the GeneChip® human genome U133 plus 2.0 microarray also are available at www.ncbi.nlm.nih.gov and genecards.weizmann.ac.il.

806 genes were found to be differentially expressed between inflammatory acne lesions and normal skin controls at day 1 among 17 patients enrolled in the study (Table 3; p<0.05, FDR <0.05). Table 4 lists the general gene ontology attributes of the differentially expressed genes between inflammatory acne lesions and normal skin controls at day 1 as determined by FuncAssociate 2.0 algorithm. A similar gene ontology profile was observed for genes that were differentially express in IN versus Duac treatment. Table 5 lists the number of genes whose expression profile correlated with Duac treatment in over-represented GO attributes. The similarity in gene ontology profiles suggests that Duac treatment functioned to restore acne lesions back to normal physiology. For example, it was found that tissue inhibitor of metalloproteinase 3 (TIMP-3) (Gene ontology group 0004866, endopeptidase inhibitor activity) exhibited lower expression in inflammatory lesions compared to normal skin. Expression of TIMP-3 in inflammatory lesions is elevated during the time course of Duac treatment. Thus, the expression profile of TIMP-3 correlates with Duac treatment of inflammatory acne lesions. In another example, Defensin (34 which is known to be over-expressed in inflammatory acne lesions compared to normal skin, exhibited decreased expression over time during Duac treatment. Thus, Defensin 134 is a candidate biomarker for monitoring the progress of acne lesions with Duac treatment.

A self-organizing map (SOM) analysis was performed to provide a non-hierarchical unsupervised and iterative approach to grouping genes with similar expression profiled. The 806 differentially expressed genes were subjected to SOM at 4×3 nodes with 10,000 iterations. 270 of the 806 genes were found to be overexpressed in inflammatory acne lesions compared to normal skin at day 1 and that decreased during Duac treatment. 126 genes of these 270 were selected based on genes that were most differentially expressed in acne lesions before and after 8 weeks of Duac treatment (Table 6). The top biological functions of these 126 genes were dermatological diseases and conditions (12 genes), inflammatory response/disease (7 genes), cell death (32 genes), cell cycles (16 genes) and cellular growth and proliferation (30 genes). Table 6 lists that average expression data for these 126 gene between inflammatory acne lesions and normal skin controls at day 1 and Duac treatment at week 2, week 5 and week 8 time points.

261 of the 806 genes were found to be underexpressed in inflammatory acne lesions compared to normal skin at day 1 and that increased during Duac treatment. 119 genes of these 261 were selected based on genes that were most differentially expressed in acne lesions before and after 8 weeks of Duac treatment (Table 7).

From the selected, 20 genes were further selected from the above described groups as biomarkers for response to Duac treatment based on both biological function and expression profiles (Table 8). Table 9 represents a subset of the genes listed in Table 8.

TABLE 3
806 differentially expressed genes between inflammatory acne lesions and normal skin controls at day 1

| Acne-I N-NS- | Acne inflammatory | | Normal skin | | | |
|---|---|---|---|---|---|---|
| 806 genelist | lesions (IN) | | controls (NS) | | IN/NS | |
| probeset ID | Mean | StdErr | Mean | StdErr | fold | Description |
| 211970_x_at | 6161.06 | 815.67 | 8689.76 | 445.48 | 0.71 | actin, gamma 1 |
| 33322_i_at | 11767.77 | 1276.16 | 17080.71 | 425.24 | 0.69 | stratifin |
| 210715_s_at | 732.82 | 176.28 | 1089.12 | 148.06 | 0.67 | serine peptidase inhibitor, Kunitz type, 2 |
| 216319_at | 371.41 | 43.96 | 232.35 | 22.34 | 1.60 | |
| 211565_at | 1213.00 | 226.17 | 459.65 | 60.17 | 2.64 | SH3-domain GRB2-like 3 |
| 212363_x_at | 1274.88 | 194.30 | 1811.41 | 125.51 | 0.70 | actin, gamma 1 |
| 211940_x_at | 2347.53 | 526.97 | 3638.29 | 411.10 | 0.65 | H3 histone, family 3A; H3 histone, family 3A |
| | | | | | | pseudogene |
| 200775_s_at | 758.29 | 83.34 | 575.41 | 115.40 | 1.32 | heterogeneous nuclear ribonucleoprotein K |
| 201550_x_at | 3094.35 | 475.22 | 4616.71 | 287.76 | 0.67 | actin, gamma 1 |
| 208755_x_at | 2247.41 | 391.72 | 3203.53 | 298.13 | 0.70 | H3 histone, family 3A |
| 1565666_s_at | 3310.76 | 502.13 | 1181.47 | 193.12 | 2.80 | mucin 6, gastric |
| 236243_at | 383.35 | 61.49 | 212.35 | 36.80 | 1.81 | Zinc finger, CCHC domain containing 6 |
| 200906_s_at | 726.47 | 291.26 | 1425.53 | 296.02 | 0.51 | palladin |
| 213828_x_at | 2691.18 | 514.37 | 4020.59 | 397.27 | 0.67 | H3 histone, family 3A; H3 histone, family 3A |
| | | | | | | pseudogene |
| 217491_x_at | 1603.59 | 441.37 | 2722.35 | 410.62 | 0.59 | cytochrome c oxidase subunit Vllc |
| 218392_x_at | 324.76 | 42.13 | 184.65 | 16.28 | 1.76 | sideroflexin 1 |
| 212082_s_at | 5065.94 | 1032.67 | 7876.82 | 715.24 | 0.64 | myosin, light polypeptide 6, alkali, smooth muscle |
| | | | | | | and non-muscle |
| 212988_x_at | 6051.24 | 862.12 | 8731.76 | 521.84 | 0.69 | actin, gamma 1 |
| 217557_s_at | 1158.12 | 167.04 | 400.82 | 67.61 | 2.89 | |
| 33323_r_at | 10049.82 | 1091.82 | 15052.35 | 432.11 | 0.67 | stratifin |
| 1557135_at | 523.88 | 64.36 | 293.71 | 27.85 | 1.78 | |
| 214549_x_at | 7476.94 | 1509.39 | 12567.77 | 1194.56 | 0.59 | small proline-rich protein 1A |
| 210378_s_at | 129.53 | 15.84 | 93.35 | 2.43 | 1.39 | Sjogren's syndrome nuclear autoantigen 1 |
| 200801_x_at | 10728.00 | 1055.98 | 14468.59 | 585.40 | 0.74 | actin, beta |
| 209492_x_at | 1685.35 | 473.43 | 4013.59 | 613.09 | 0.42 | ATP synthase, H+ transporting, mitochondrial FO |
| | | | | | | complex, subunit e |
| 217579_x_at | 723.53 | 91.78 | 1805.76 | 342.01 | 0.40 | ADP-ribosylation factor-like 6 interacting protein 2 |
| 217719_at | 729.71 | 232.31 | 1354.59 | 171.99 | 0.54 | eukaryotic translation initiation factor 3, subunit 6 |
| | | | | | | interacting protein |
| 206453_s_at | 3863.12 | 814.44 | 6398.65 | 735.17 | 0.60 | NDRG family member 2 |
| 204254_s_at | 3507.94 | 971.02 | 5630.29 | 713.92 | 0.62 | vitamin D (1,25-dihydroxyvitamin D3) receptor |
| 212089_at | 1658.47 | 408.00 | 2730.65 | 389.03 | 0.61 | lamin A/C |
| 1558154_at | 434.71 | 122.38 | 918.94 | 189.68 | 0.47 | Lethal giant larvae homolog 2 (Drosophila) |
| 211050_x_at | 2264.12 | 249.88 | 1140.06 | 69.18 | 1.99 | Similar to general transcription factor II, i isoform 1; |
| | | | | | | BTK-associated protein, 135 kD; Williams-Beuren |
| | | | | | | syndrome chromosome region 6; Bruton tyrosine |
| | | | | | | kinase-associated protein 135; Similar to general |
| | | | | | | transcription factor II, i isoform 1; BTK-associated |
| | | | | | | protein, 135 kD; Williams-Beuren syndrome |
| | | | | | | chromosome region 6; Bruton tyrosine kinase- |
| | | | | | | associated protein 135; Similar to hypothetical |
| | | | | | | protein LOC284701; Similar to hypothetical protein |
| | | | | | | LOC284701 |
| 231809_x_at | 919.94 | 438.80 | 171.29 | 41.80 | 5.37 | programmed cell death 7 |
| 211074_at | 11948.77 | 1669.11 | 17063.88 | 896.25 | 0.70 | folate receptor 1 (adult); folate receptor 1 (adult) |
| 216438_s_at | 4675.41 | 1097.26 | 6173.18 | 612.50 | 0.76 | thymosin, beta 4, X-linked; thymosin-like 3 |
| 208676_s_at | 466.00 | 75.17 | 791.82 | 96.07 | 0.59 | proliferation-associated 2G4, 38 kDa |
| 236259_at | 1249.12 | 131.21 | 809.94 | 96.49 | 1.54 | serine/threonine kinase 4 |
| 208904_s_at | 1324.41 | 299.84 | 2958.00 | 398.70 | 0.45 | ribosomal protein S28 |
| 208687_x_at | 2037.41 | 486.08 | 3195.76 | 416.14 | 0.64 | heat shock 70 kDa protein 8 |
| 201001_s_at | 1928.24 | 514.85 | 1383.71 | 332.71 | 1.39 | ubiquitin-conjugating enzyme E2 variant 1; |
| | | | | | | ubiquitin-conjugating enzyme E2 variant 1 |
| 200741_s_at | 991.24 | 159.12 | 766.94 | 136.57 | 1.29 | ribosomal protein S27 (metallopanstimulin 1) |
| 213826_s_at | 2132.76 | 424.79 | 3100.35 | 346.10 | 0.69 | |
| 200926_at | 4809.12 | 850.09 | 6545.88 | 613.92 | 0.73 | ribosomal protein S23 |
| 200833_s_at | 1105.71 | 192.77 | 768.71 | 136.93 | 1.44 | RAP1B, member of RAS oncogene family |
| 212242_at | 1052.18 | 298.21 | 1625.41 | 274.26 | 0.65 | tubulin, alpha 1 (testis specific) |
| 200077_s_at | 812.88 | 181.75 | 1210.65 | 149.80 | 0.67 | ornithine decarboxylase antizyme 1; ornithine |
| | | | | | | decarboxylase antizyme 1 |
| 1566145_s_at | 2189.24 | 418.24 | 806.35 | 145.32 | 2.71 | SH3-domain GRB2-like 3 |
| 201437_s_at | 668.41 | 70.02 | 509.12 | 78.09 | 1.31 | eukaryotic translation initiation factor 4E |
| 200673_at | 1247.65 | 275.47 | 2091.24 | 259.23 | 0.60 | lysosomal-associated protein transmembrane 4 alpha |
| 235514_at | 3876.29 | 835.30 | 5320.06 | 499.46 | 0.73 | Skin ASpartic Protease |
| 211296_x_at | 2734.88 | 551.49 | 4343.41 | 368.21 | 0.63 | ubiquitin C |
| 222444_at | 310.59 | 43.25 | 751.24 | 113.15 | 0.41 | armadillo repeat containing, X-linked 3 |
| 209126_x_at | 2667.53 | 830.45 | 4242.59 | 562.57 | 0.63 | keratin 6B |
| 209118_s_at | 4060.35 | 596.55 | 6386.47 | 402.88 | 0.64 | tubulin, alpha 3 |
| 208980_s_at | 4096.77 | 775.20 | 6678.47 | 577.39 | 0.61 | ubiquitin C |
| 229630_s_at | 1539.12 | 220.57 | 720.82 | 127.55 | 2.14 | Wilms tumor 1 associated protein |
| 216428_x_at | 416.41 | 70.70 | 193.35 | 34.10 | 2.15 | hypothetical gene FLJ00060 |
| 236346_at | 208.18 | 45.27 | 99.18 | 9.00 | 2.10 | Zinc finger protein 83 (HPF1) |
| 213796_at | 9849.35 | 1780.47 | 16667.53 | 1081.58 | 0.59 | small proline-rich protein 1A |
| 232220_at | 1333.24 | 693.64 | 210.59 | 67.65 | 6.33 | |
| 213032_at | 1390.65 | 377.90 | 3984.53 | 539.16 | 0.35 | Nuclear factor I/B |
| 203725_at | 1901.12 | 530.05 | 226.24 | 58.64 | 8.40 | growth arrest and DNA-damage-inducible, alpha |
| 208539_x_at | 4753.12 | 820.95 | 1017.59 | 199.23 | 4.67 | small proline-rich protein 2B |
| 238967_at | 126.65 | 10.29 | 493.00 | 152.15 | 0.26 | Claudin 1 |
| 226006_at | 3036.00 | 854.76 | 5022.41 | 799.82 | 0.60 | |
| 224841_x_at | 894.76 | 412.47 | 2052.53 | 660.13 | 0.44 | growth arrest-specific 5 |
| 39248_at | 8999.88 | 1554.18 | 14515.59 | 523.15 | 0.62 | aquaporin 3 |
| 201829_at | 1045.29 | 244.16 | 1912.47 | 319.40 | 0.55 | neuroepithelial cell transforming gene 1 |
| 218050_at | 816.24 | 114.47 | 459.41 | 74.68 | 1.78 | ubiquitin-fold modifier 1 |
| 211995_x_at | 744.76 | 211.56 | 1222.59 | 156.41 | 0.61 | actin, gamma 1 |
| 214143_x_at | 3188.59 | 878.03 | 3890.88 | 486.23 | 0.82 | ribosomal protein L24; solute carrier family 36 |
| | | | | | | (proton/amino acid symporter), member 2 |
| 218045_x_at | 555.41 | 338.26 | 1200.00 | 234.44 | 0.46 | parathymosin |
| 208949_s_at | 3910.35 | 789.40 | 6335.18 | 740.30 | 0.62 | lectin, galactoside-binding, soluble, 3 (galectin 3); |
| | | | | | | galectin-3 internal gene |
| 233993_at | 1371.47 | 250.19 | 629.71 | 118.40 | 2.18 | SIB 297 intestinal mucin (MUC3) |
| 201631_s_at | 6333.35 | 1195.43 | 11354.35 | 731.06 | 0.56 | immediate early response 3 |
| 228477_at | 991.82 | 256.89 | 1837.12 | 346.06 | 0.54 | Hypothetical protein FLJ10154 |
| 227404_s_at | 3793.35 | 840.20 | 6696.00 | 927.38 | 0.57 | Early growth response 1 |
| 202464_s_at | 3694.12 | 679.30 | 1245.88 | 246.89 | 2.97 | 6-phosphofructo-2-kinase/fructose-2,6-biphosphatase 3 |
| 212236_x_at | 4576.71 | 1516.08 | 10220.71 | 1599.54 | 0.45 | keratin 17 |
| 200081_s_at | 1600.88 | 387.83 | 2401.24 | 348.75 | 0.67 | ribosomal protein S6; ribosomal protein S6 |
| 228993_s_at | 3799.24 | 884.32 | 6934.53 | 787.25 | 0.55 | hypothetical protein LOC92482 |
| 218182_s_at | 1502.76 | 496.87 | 2812.24 | 492.08 | 0.53 | claudin 1 |
| 227621_at | 1741.24 | 271.30 | 969.53 | 222.64 | 1.80 | Wilms tumor 1 associated protein |
| 214580_x_at | 2056.18 | 439.29 | 3292.71 | 337.39 | 0.62 | keratin 6A; keratin 6B; keratin 6C; keratin 6E |
| 211617_at | 836.41 | 165.56 | 377.35 | 186.38 | 2.22 | aldolase A, fructose-bisphosphate pseudogene 2; |
| | | | | | | aldolase A, fructose-bisphosphate pseudogene 2 |
| 210453_x_at | 1635.94 | 433.74 | 2253.35 | 270.07 | 0.73 | ATP synthase, H+ transporting, mitochondrial F0 |
| | | | | | | complex, subunit g |
| 226465_s_at | 2843.53 | 628.60 | 5926.06 | 728.61 | 0.48 | SON DNA binding protein |
| 201065_s_at | 1311.94 | 264.33 | 1992.53 | 186.82 | 0.66 | general transcription factor II, i; general transcription |
| | | | | | | factor II, pseudogene 1 |
| 214257_s_at | 729.94 | 245.17 | 1129.71 | 174.17 | 0.65 | SEC22 vesicle trafficking protein-like 1 (S. cerevisiae) |
| 214374_s_at | 2995.41 | 862.20 | 4556.06 | 526.80 | 0.66 | PTPRF interacting protein, binding protein 1 (liprin |
| | | | | | | beta 1) |
| 208692_at | 3819.53 | 1047.39 | 5573.00 | 806.53 | 0.69 | ribosomal protein S3 |
| 218200_s_at | 921.24 | 297.96 | 1535.29 | 270.97 | 0.60 | NADH dehydrogenase (ubiquinone) 1 beta |
| | | | | | | subcomplex, 2, 8 kDa |
| 205157_s_at | 4596.94 | 1779.93 | 9482.94 | 1650.07 | 0.48 | keratin 17 |
| 205185_at | 5749.00 | 1160.42 | 11126.24 | 860.33 | 0.52 | serine peptidase inhibitor, Kazal type 5 |
| 212317_at | 462.41 | 101.83 | 669.88 | 95.57 | 0.69 | transportin 3 |
| 222474_s_at | 544.71 | 117.54 | 763.59 | 134.12 | 0.71 | translocase of outer mitochondrial membrane 22 |
| | | | | | | homolog (yeast) |
| 200815_s_at | 1083.65 | 233.05 | 2617.35 | 224.98 | 0.41 | platelet-activating factor acetylhydrolase, isoform lb, |
| | | | | | | alpha subunit 45 kDa |
| 229606_at | 892.29 | 131.59 | 1654.53 | 233.09 | 0.54 | Protein phosphatase 3 (formerly 2B), catalytic |
| | | | | | | subunit, alpha isoform (calcineurin A alpha) |
| 211927_x_at | 2250.35 | 504.12 | 3324.35 | 448.54 | 0.68 | eukaryotic translation elongation factor 1 gamma |
| 201694_s_at | 1556.94 | 358.06 | 2551.00 | 376.07 | 0.61 | early growth response 1 |
| 208655_at | 549.41 | 151.71 | 1228.35 | 243.17 | 0.45 | Cyclin I |
| 224602_at | 1429.88 | 390.53 | 2964.35 | 420.86 | 0.48 | HCV F-transactivated protein 1 |
| 211345_x_at | 2575.82 | 575.50 | 3756.59 | 496.57 | 0.69 | eukaryotic translation elongation factor 1gamma |
| 202935_s_at | 1514.29 | 402.68 | 1999.00 | 249.70 | 0.76 | SRY (sex determining region Y)-box 9 (campomelic |
| | | | | | | dysplasia, autosomal sex-reversal) |
| 1558924_s_at | 1514.12 | 401.42 | 2620.94 | 337.38 | 0.58 | restin (Reed-Steinberg cell-expressed intermediate |
| | | | | | | filament-associated protein) |
| 206654_s_at | 608.06 | 171.62 | 300.88 | 76.36 | 2.02 | polymerase (RNA) III (DNA directed) polypeptide G |
| | | | | | | (32 kD) |
| 211986_at | 1004.12 | 288.71 | 1728.00 | 290.53 | 0.58 | AHNAK nucleoprotein (desmoyokin) |
| 1566146_x_at | 1012.53 | 155.18 | 319.82 | 59.54 | 3.17 | SH3-domain GRB2-like 3 |
| 224239_at | 423.41 | 50.34 | 204.76 | 20.81 | 2.07 | defensin, beta 103A |
| 224741_x_at | 987.82 | 448.33 | 2073.82 | 635.96 | 0.48 | growth arrest-specific 5 |
| 206642_at | 1226.71 | 311.36 | 2148.71 | 271.27 | 0.57 | desmoglein 1 |
| 201131_s_at | 1146.71 | 267.77 | 2167.29 | 206.10 | 0.53 | cadherin 1, type 1, E-cadherin (epithelial) |
| 200641_s_at | 933.12 | 269.27 | 1246.06 | 204.82 | 0.75 | tyrosine 3-monooxygenase/tryptophan 5- |
| | | | | | | monooxygenase activation protein, zeta polypeptide |
| 1566144_at | 1035.06 | 167.62 | 360.59 | 83.07 | 2.87 | SH3-domain GRB2-like 3 |
| 202712_s_at | 1783.59 | 538.88 | 2680.94 | 447.13 | 0.67 | creatine kinase, mitochondrial 1B; creatine kinase, |
| | | | | | | mitochondrial 1A |
| 208746_x_at | 1687.88 | 422.90 | 2555.47 | 314.78 | 0.66 | ATP synthase, H+ transporting, mitochondrial F0 |
| | | | | | | complex, subunit g |
| 217769_s_at | 1767.47 | 425.42 | 3039.18 | 396.28 | 0.58 | chromosome 13 open reading frame 12 |
| 200762_at | 1448.76 | 340.69 | 2023.35 | 258.31 | 0.72 | dihydropyrimidinase-like 2 |
| 219762_s_at | 425.53 | 136.79 | 614.59 | 120.90 | 0.69 | ribosomal protein L36 |
| 211962_s_at | 638.94 | 182.39 | 1172.53 | 179.55 | 0.54 | zinc finger protein 36, C3H type-like 1 |
| 213680_at | 6532.88 | 1244.90 | 11383.94 | 806.72 | 0.57 | keratin 6B |
| 212593_s_at | 3165.53 | 695.46 | 6193.71 | 693.60 | 0.51 | programmed cell death 4 (neoplastic transformation |
| | | | | | | inhibitor) |
| 225667_s_at | 368.65 | 71.13 | 854.29 | 139.26 | 0.43 | family with sequence similarity 84, member A; |
| | | | | | | hypothetical LOC400944 |
| 200689_x_at | 2642.35 | 600.55 | 3835.41 | 546.08 | 0.69 | eukaryotic translation elongation factor 1 gamma |
| 200057_s_at | 630.76 | 259.68 | 904.06 | 183.30 | 0.70 | non-POU domain containing, octamer-binding; non- |
| | | | | | | POU domain containing, octamer-binding |
| 224889_at | 569.88 | 235.41 | 1196.94 | 221.88 | 0.48 | forkhead box O3A |
| 235281_x_at | 1793.06 | 399.83 | 3133.29 | 343.42 | 0.57 | AHNAK nucleoprotein (desmoyokin) |
| 219380_x_at | 210.41 | 39.34 | 96.71 | 7.66 | 2.18 | Polymerase (DNA directed), eta |
| 201592_at | 465.35 | 156.95 | 1045.71 | 228.63 | 0.45 | eukaryotic translation initiation factor 3, subunit 3 |
| | | | | | | gamma, 40 kDa |
| 227052_at | 606.82 | 159.57 | 1243.94 | 252.17 | 0.49 | Hypothetical protein LOC201895 |
| 215832_x_at | 102.47 | 23.17 | 65.53 | 3.08 | 1.56 | phosphatidylinositol binding clathrin assembly |
| | | | | | | protein |
| 200655_s_at | 1945.94 | 505.16 | 2919.82 | 376.69 | 0.67 | calmodulin 1 (phosphorylase kinase, delta) |
| 207335_x_at | 289.76 | 95.56 | 548.18 | 86.68 | 0.53 | ATP synthase, H+ transporting, mitochondrial F0 |
| | | | | | | complex, subunit e |
| 1555743_s_at | 269.41 | 33.83 | 144.65 | 17.20 | 1.86 | NADH dehydrogenase (ubiquinone) 1 beta |
| 201226_at | 277.00 | 115.52 | 518.47 | 109.07 | 0.53 | subcomplex, 8, 19 kDa |
| 239377_at | 1459.12 | 361.17 | 3120.76 | 416.34 | 0.47 | hypothetical protein MGC11102 |
| 1553602_at | 3601.82 | 942.85 | 10080.88 | 885.37 | 0.36 | small breast epithelial mucin |
| 231548_at | 573.88 | 226.60 | 1094.47 | 196.00 | 0.52 | Forkhead box O3A |
| 224559_at | 1654.82 | 423.65 | 2792.82 | 236.43 | 0.59 | metastasis associated lung adenocarcinoma transcript |
| | | | | | | 1 (non-coding RNA) |
| 204427_s_at | 1504.71 | 582.00 | 1878.35 | 273.05 | 0.80 | transmembrane emp24 domain trafficking protein 2 |
| 229353_s_at | 1482.47 | 546.34 | 2778.88 | 562.30 | 0.53 | nuclear casein kinase and cyclin-dependent kinase |
| | | | | | | substrate 1 |
| 209691_s_at | 226.94 | 71.69 | 480.29 | 118.65 | 0.47 | docking protein 4 |
| 1563560_at | 1117.94 | 291.96 | 1935.24 | 212.92 | 0.58 | AHNAK nucleoprotein (desmoyokin) |
| 231733_at | 1326.94 | 393.41 | 3635.76 | 557.95 | 0.36 | ICEBERG caspase-1 inhibitor |
| 203962_s_at | 1203.71 | 367.13 | 2430.47 | 381.28 | 0.50 | nebulette |
| 214939_x_at | 291.18 | 57.08 | 739.41 | 158.75 | 0.39 | myeloid/lymphoid or mixed-lineage leukemia |
| | | | | | | (trithorax homolog, Drosophila); translocated to, 4 |
| 205209_at | 216.35 | 55.75 | 494.12 | 61.51 | 0.44 | activin A receptor, type IB |
| 205812_s_at | 1016.35 | 275.90 | 1619.47 | 222.30 | 0.63 | transmembrane emp24 protein transport domain |
| | | | | | | containing 9 |
| 212515_s_at | 137.00 | 20.83 | 215.88 | 28.09 | 0.63 | DEAD (Asp-Glu-Ala-Asp) box polypeptide 3, X- |
| | | | | | | linked |
| 210589_s_at | 404.12 | 102.86 | 112.82 | 33.95 | 3.58 | glucosidase, beta; acid (includes glucosylceramidase); |
| | | | | | | glucosidase, beta; acid, pseudogene |
| 202777_at | 624.29 | 140.83 | 1450.88 | 356.15 | 0.43 | soc-2 suppressor of clear homolog (C. elegans) |
| 206665_s_at | 913.41 | 356.18 | 2024.65 | 534.54 | 0.45 | BCL2-like 1 |
| 207573_x_at | 701.47 | 181.98 | 951.06 | 111.58 | 0.74 | ATP synthase, H+ transporting, mitochondrial F0 |
| | | | | | | complex, subunit g |
| 236119_s_at | 4203.82 | 730.91 | 603.65 | 207.85 | 6.96 | small proline-rich protein 2G |
| 201134_x_at | 1289.18 | 379.79 | 2073.18 | 338.66 | 0.62 | cytochrome c oxidase subunit VIIc |
| 200907_s_at | 1117.29 | 264.48 | 2909.00 | 553.25 | 0.38 | palladin |
| 200965_s_at | 2919.71 | 687.14 | 5332.88 | 786.15 | 0.55 | actin binding LIM protein 1 |
| 202163_s_at | 339.71 | 116.45 | 724.00 | 151.26 | 0.47 | CCR4-NOT transcription complex, subunit 8 |
| 227964_at | 571.53 | 144.50 | 1000.29 | 165.44 | 0.57 | FKSG44 gene |
| 211662_s_at | 1165.24 | 462.59 | 1438.18 | 253.98 | 0.81 | voltage-dependent anion channel 2; voltage- |
| | | | | | | dependent anion channel 2 |
| 201296_s_at | 218.00 | 69.60 | 497.06 | 138.16 | 0.44 | WD repeat and SOCS box-containing 1 |
| 209800_at | 1499.35 | 635.37 | 3047.82 | 639.97 | 0.49 | keratin 16 (focal non-epidermolytic palmoplantar |
| | | | | | | keratoderma) |
| 213260_at | 769.53 | 222.19 | 1267.41 | 225.93 | 0.61 | Forkhead box C1 |
| 227492_at | 1136.41 | 347.23 | 2082.65 | 357.41 | 0.55 | Transcribed locus, moderately similar to |
| | | | | | | NP_689672.2 hypothetical protein MGC45438 |
| | | | | | | [Homo sapiens] |
| 213911_s_at | 406.47 | 166.07 | 759.53 | 199.92 | 0.54 | H2A histone family, member Z |
| 218330_s_at | 258.59 | 60.43 | 661.53 | 154.68 | 0.39 | neuron navigator 2 |
| 223000_s_at | 2131.65 | 626.38 | 3614.06 | 498.58 | 0.59 | F11 receptor |
| 206595_at | 4382.53 | 1244.50 | 12790.77 | 1244.78 | 0.34 | cystatin E/M |
| 213726_x_at | 2715.65 | 763.18 | 4101.41 | 614.52 | 0.66 | tubulin, beta, 2 |
| 220431_at | 380.76 | 118.67 | 1116.88 | 366.51 | 0.34 | transmembrane protease, serine 11E |
| 223338_s_at | 480.71 | 122.49 | 894.76 | 144.54 | 0.54 | ATPase inhibitory factor 1 |
| 208693_s_at | 471.59 | 85.34 | 317.53 | 71.34 | 1.49 | glycyl-tRNA synthetase |
| 204517_at | 818.59 | 237.07 | 1176.53 | 216.43 | 0.70 | peptidylprolyl isomerase C (cyclophilin C) |
| 1552620_at | 3340.47 | 906.54 | 8518.41 | 680.18 | 0.39 | small proline rich protein 4 |
| 201005_at | 1648.29 | 485.73 | 3006.53 | 380.43 | 0.55 | CD9 antigen (p24) |
| 218067_s_at | 530.24 | 173.53 | 1001.41 | 209.35 | 0.53 | hypothetical protein FLJ10154 |
| 219410_at | 3884.82 | 819.55 | 6826.12 | 551.26 | 0.57 | transmembrane protein 45A |
| 206605_at | 1079.59 | 334.27 | 2703.41 | 410.65 | 0.40 | 26 serine protease |
| 229027_at | 477.35 | 93.24 | 213.82 | 55.11 | 2.23 | Protein phosphatase 1A (formerly 2C), magnesium- |
| | | | | | | dependent, alpha isoform |
| 203744_at | 235.06 | 34.41 | 110.53 | 15.52 | 2.13 | high-mobility group box 3 |
| 209283_at | 1800.06 | 456.70 | 3023.71 | 416.19 | 0.60 | crystallin, alpha B |
| 224867_at | 796.00 | 299.28 | 1271.76 | 216.86 | 0.63 | chromosome 1 open reading frame 151 |
| 1554678_s_at | 859.59 | 309.23 | 1672.82 | 261.36 | 0.51 | heterogeneous nuclear ribonucleoprotein D-like |
| 208901_s_at | 503.88 | 107.48 | 221.12 | 42.89 | 2.28 | topoisomerase (DNA) I |
| 1552664_at | 152.12 | 36.57 | 71.18 | 9.43 | 2.14 | folliculin |
| 203123_s_at | 1268.88 | 341.99 | 1947.24 | 347.70 | 0.65 | solute carrier family 11 (proton-coupled divalent |
| | | | | | | metal ion transporters), member 2 |
| 210074_at | 6861.94 | 1542.41 | 12024.59 | 1167.21 | 0.57 | cathepsin L2 |
| 207114_at | 1430.88 | 403.81 | 2513.88 | 416.96 | 0.57 | lymphocyte antigen 6 complex, locus G6C |
| 220167_s_at | 1376.24 | 339.73 | 628.82 | 177.94 | 2.19 | TP53TG3 protein |
| 203430_at | 1139.94 | 344.12 | 2021.71 | 277.75 | 0.56 | heme binding protein 2 |
| 200709_at | 636.88 | 161.20 | 1156.94 | 200.17 | 0.55 | FK506 binding protein 1A, 12 kDa |
| 200606_at | 982.00 | 259.47 | 1705.47 | 220.55 | 0.58 | desmoplakin |
| 237120_at | 3503.35 | 1064.11 | 5350.47 | 660.63 | 0.65 | keratin 1B |
| 203798_s_at | 832.59 | 370.40 | 2534.41 | 437.81 | 0.33 | visinin-like 1 |
| 214370_at | 6947.35 | 1213.08 | 977.71 | 308.71 | 7.11 | S100 calcium binding protein AS (calgranulin A) |
| 205178_s_at | 1144.53 | 265.80 | 577.06 | 154.17 | 1.98 | retinoblastoma binding protein 6 |
| 224571_at | 684.24 | 264.41 | 2478.41 | 471.74 | 0.28 | interferon regulatory factor 2 binding protein 2 |
| 202053_s_at | 881.47 | 235.37 | 3964.59 | 605.40 | 0.22 | aldehyde dehydrogenase 3 family, member A2 |
| 214119_s_at | 285.18 | 106.08 | 840.59 | 136.92 | 0.34 | FK506 binding protein 1A, 12 kDa |
| 201160_s_at | 2036.59 | 594.49 | 2760.24 | 449.14 | 0.74 | cold shock domain protein A |
| 225345_s_at | 264.47 | 82.01 | 1180.12 | 287.01 | 0.22 | F-box protein 32 |
| 225629_s_at | 382.41 | 100.54 | 2078.00 | 398.38 | 0.18 | zinc finger and BTB domain containing 4 |
| 218739_at | 1197.53 | 300.81 | 1714.71 | 311.13 | 0.70 | abhydrolase domain containing 5 |
| 233330_s_at | 950.71 | 246.47 | 377.41 | 170.18 | 2.52 | Immunoglobulin kappa chain variable region, (V1-Vk |
| | | | | | | gene), from multiple myeloma: MM-8k |
| 201525_at | 2040.82 | 818.88 | 4838.76 | 958.06 | 0.42 | apolipoprotein D |
| 212566_at | 997.59 | 276.98 | 2318.00 | 501.27 | 0.43 | microtubule-associated protein 4 |
| 224570_s_at | 475.65 | 209.93 | 1139.18 | 209.07 | 0.42 | interferon regulatory factor 2 binding protein 2 |
| 232082_x_at | 1203.06 | 187.59 | 174.18 | 44.89 | 6.91 | small proline-rich protein 3 |
| 205249_at | 658.24 | 178.12 | 1409.71 | 247.02 | 0.47 | early growth response 2 (Krox-20 homolog, |
| | | | | | | Drosophila) |
| 205778_at | 1772.24 | 562.37 | 3274.59 | 655.25 | 0.54 | kallikrein 7 (chymotryptic, stratum corneum) |
| 214198_s_at | 308.76 | 95.40 | 692.41 | 149.53 | 0.45 | DiGeorge syndrome critical region gene 2 |
| 207564_x_at | 407.24 | 172.26 | 794.65 | 281.09 | 0.51 | O-linked N-acetylglucosamine (GlcNAc) transferase |
| | | | | | | (UDP-N-acetylglucosamine: polypeptide-N- |
| | | | | | | acetylglucosaminyl transferase) |
| 201667_at | 3988.12 | 836.39 | 6007.12 | 681.00 | 0.66 | gap junction protein, alpha 1, 43 kDa (connexin 43) |
| 201662_s_at | 535.65 | 135.45 | 257.06 | 55.19 | 2.08 | acyl-CoA synthetase long-chain family member 3 |
| 1558378_a_at | 3328.06 | 1113.57 | 5608.00 | 607.00 | 0.59 | chromosome 14 open reading frame 78 |
| 201260_s_at | 977.18 | 244.05 | 1448.24 | 232.05 | 0.67 | synaptophysin-like 1 |
| 224328_s_at | 5539.82 | 859.60 | 1006.47 | 218.75 | 5.50 | late cornified envelope 3D; late cornified envelope |
| | | | | | | 3D |
| 240331_at | 1051.82 | 153.23 | 529.18 | 158.30 | 1.99 | Bone morphogenetic protein receptor, type 1B |
| 222549_at | 785.41 | 211.07 | 1977.76 | 407.12 | 0.40 | claudin 1 |
| 202917_s_at | 6670.24 | 1265.67 | 929.76 | 342.63 | 7.17 | S100 calcium binding protein A8 (calgranulin A) |
| 1555961_a_at | 1124.94 | 357.17 | 1752.59 | 246.55 | 0.64 | histidine triad nucleotide binding protein 1 |
| 207358_x_at | 353.76 | 92.87 | 617.76 | 114.93 | 0.57 | microtubule-actin crosslinking factor 1 |
| 201615_x_at | 307.88 | 114.27 | 935.76 | 197.72 | 0.33 | caldesmon 1 |
| 201161_s_at | 1398.59 | 394.67 | 2427.00 | 448.77 | 0.58 | cold shock domain protein A |
| 212322_at | 1155.12 | 369.22 | 1501.59 | 280.58 | 0.77 | sphingosine-1-phosphate lyase 1 |
| 206032_at | 1878.65 | 496.24 | 3678.18 | 420.90 | 0.51 | desmocollin 3 |
| 1554671_a_at | 411.35 | 153.49 | 968.06 | 259.28 | 0.42 | serine/arginine repetitive matrix 2 |
| 201223_s_at | 444.24 | 118.95 | 1383.47 | 266.74 | 0.32 | RAD23 homolog B (S. cerevisiae) |
| 205807_s_at | 936.18 | 276.28 | 1553.94 | 254.18 | 0.60 | tuftelin 1 |
| 200964_at | 287.47 | 113.95 | 524.18 | 151.56 | 0.55 | ubiquitin-activating enzyme El (A1S9T and BN75 |
| | | | | | | temperature sensitivity complementing) |
| 217918_at | 558.24 | 187.77 | 927.94 | 160.78 | 0.60 | dynein, cytoplasmic, light polypeptide 2A |
| 200090_at | 229.18 | 71.18 | 518.59 | 100.68 | 0.44 | farnesyltransferase, CAAX box, alpha; |
| | | | | | | farnesyltransferase, CAAX box, alpha |
| 200897_s_at | 617.12 | 195.74 | 1543.71 | 331.87 | 0.40 | palladin |
| 225615_at | 463.47 | 150.81 | 1038.29 | 208.05 | 0.45 | hypothetical protein LOC126917 |
| 230296_at | 523.59 | 132.14 | 150.65 | 33.05 | 3.48 | Chromosome 16 open reading frame 52 |
| 204426_at | 1291.12 | 415.41 | 1765.29 | 426.58 | 0.73 | transmembrane emp24 domain trafficking protein 2 |
| 226545_at | 487.82 | 180.20 | 1106.35 | 214.04 | 0.44 | CD109 antigen (Gov platelet alloantigens) |
| 208892_s_at | 383.06 | 122.34 | 800.47 | 135.57 | 0.48 | dual specificity phosphatase 6 |
| 217717_s_at | 181.53 | 64.41 | 623.94 | 146.39 | 0.29 | tyrosine 3-monooxygenase/tryptophan 5- |
| | | | | | | monooxygenase activation protein, beta polypeptide |
| 206116_s_at | 1097.47 | 604.00 | 3447.29 | 798.75 | 0.32 | tropomyosin 1 (alpha) |
| 209845_at | 493.12 | 98.33 | 320.53 | 96.87 | 1.54 | makorin, ring finger protein, 1 |
| 241199_x_at | 388.12 | 81.93 | 81.00 | 17.17 | 4.79 | developmental pluripotency associated 4 |
| 225864_at | 582.12 | 183.97 | 914.18 | 174.90 | 0.64 | family with sequence similarity 84, member B |
| 217744_s_at | 993.94 | 254.57 | 1772.29 | 248.92 | 0.56 | PERP, TP53 apoptosis effector |
| 203395_s_at | 68.76 | 14.01 | 186.41 | 53.33 | 0.37 | hairy and enhancer of split 1, (Drosophila) |
| 241683_at | 1111.82 | 274.21 | 331.24 | 87.16 | 3.36 | HECT domain containing 1 |
| 212826_s_at | 483.41 | 210.13 | 931.65 | 240.44 | 0.52 | solute carrier family 25 (mitochondrial carrier; |
| | | | | | | adenine nucleotide translocator), member 6 |
| 206295_at | 904.06 | 256.02 | 1821.82 | 312.99 | 0.50 | interleukin 18 (interferon-gamma-inducing factor) |
| 217963_s_at | 848.59 | 314.82 | 1447.53 | 257.80 | 0.59 | nerve growth factor receptor (TNFRSF16) associated |
| | | | | | | protein 1 |
| 231291_at | 172.53 | 74.19 | 55.00 | 9.75 | 3.14 | Transcribed locus |
| 220413_at | 1328.35 | 386.88 | 2314.71 | 454.21 | 0.57 | solute carrier family 39 (zinc transporter), member 2 |
| 1569940_at | 586.59 | 121.25 | 180.88 | 55.80 | 3.24 | CDNA clone IMAGE: 5268379 |
| 200847_s_at | 844.12 | 235.16 | 1313.35 | 218.47 | 0.64 | transmembrane protein 66 |
| 237690_at | 382.71 | 136.34 | 844.29 | 181.76 | 0.45 | G protein-coupled receptor 115 |
| 239082_at | 645.65 | 100.04 | 277.88 | 67.09 | 2.32 | CDNA clone IMAGE: 5311370 |
| 215096_s_at | 381.24 | 105.80 | 165.41 | 68.52 | 2.30 | esterase D/formylglutathione hydrolase |
| 1554921_a_at | 1302.35 | 427.18 | 1251.71 | 129.99 | 1.04 | sciellin |
| 209326_at | 231.59 | 73.03 | 550.88 | 151.27 | 0.42 | solute carrier family 35 (UDP-galactose transporter), |
| | | | | | | member A2 |
| 225078_at | 1321.18 | 473.59 | 1651.53 | 339.75 | 0.80 | epithelial membrane protein 2 |
| 202559_x_at | 614.18 | 248.47 | 983.29 | 190.58 | 0.62 | chromosome 1 open reading frame 77 |
| 212062_at | 97.82 | 33.17 | 312.12 | 56.80 | 0.31 | ATPase, Class II, type 9A |
| 202439_s_at | 452.59 | 150.06 | 748.65 | 134.35 | 0.60 | iduronate 2-sulfatase (Hunter syndrome) |
| 214683_s_at | 292.53 | 72.11 | 93.35 | 18.96 | 3.13 | CDC-like kinase 1 |
| 209442_x_at | 235.47 | 95.80 | 579.24 | 152.03 | 0.41 | ankyrin 3, node of Ranvier (ankyrin G) |
| 214097_at | 287.00 | 76.81 | 153.71 | 71.40 | 1.87 | ribosomal protein S21 |
| 223092_at | 698.06 | 222.88 | 1293.59 | 279.87 | 0.54 | ankylosis, progressive homolog (mouse) |
| 202341_s_at | 173.82 | 68.15 | 546.82 | 129.36 | 0.32 | tripartite motif-containing 2 |
| 224573_at | 492.65 | 149.59 | 902.53 | 251.00 | 0.55 | similar to DNA segment, Chr 11, Brigham & |
| | | | | | | Women's Genetics 0434 expressed |
| 213998_s_at | 601.41 | 326.39 | 1027.41 | 328.99 | 0.59 | DEAD (Asp-Glu-Ala-Asp) box polypeptide 17 |
| 235547_at | 238.06 | 78.04 | 610.47 | 154.42 | 0.39 | Hypothetical gene CG012 |
| 226213_at | 993.94 | 342.08 | 1964.47 | 289.24 | 0.51 | v-erb-b2 erythroblastic leukemia viral oncogene |
| | | | | | | homolog 3 (avian) |
| 225912_at | 455.47 | 152.33 | 698.65 | 153.86 | 0.65 | tumor protein p53 inducible nuclear protein 1 |
| 220723_s_at | 347.41 | 151.34 | 455.29 | 131.18 | 0.76 | hypothetical protein FLJ21511 |
| 230291_s_at | 322.24 | 111.64 | 892.94 | 217.17 | 0.36 | Nuclear factor I/B |
| 204975_at | 887.65 | 307.19 | 926.47 | 166.38 | 0.96 | epithelial membrane protein 2 |
| 240715_at | 186.59 | 33.23 | 88.65 | 25.28 | 2.10 | T-box 5 |
| 212594_at | 627.76 | 190.32 | 1476.71 | 281.82 | 0.43 | programmed cell death 4 (neoplastic transformation |
| | | | | | | inhibitor) |
| 200696_s_at | 423.71 | 124.56 | 940.29 | 251.86 | 0.45 | gelsolin (amyloidosis, Finnish type) |
| 203691_at | 2097.24 | 642.51 | 185.24 | 51.46 | 11.32 | peptidase inhibitor 3, skin-derived (SKALP); |
| | | | | | | peptidase inhibitor 3, skin-derived (SKALP) |
| 201010_s_at | 519.82 | 179.41 | 1615.76 | 303.55 | 0.32 | thioredoxin interacting protein |
| 232217_at | 278.53 | 107.25 | 1439.53 | 262.00 | 0.19 | Chromosome 6 open reading frame 188 |
| 1554868_s_at | 139.76 | 19.80 | 94.47 | 22.05 | 1.48 | PEST-containing nuclear protein |
| 209260_at | 698.35 | 316.69 | 730.41 | 141.45 | 0.96 | stratifin |
| 213151_s_at | 212.47 | 59.25 | 575.53 | 134.51 | 0.37 | septin 7 |
| 241904_at | 91.18 | 14.98 | 54.76 | 5.44 | 1.66 | Lipase, hormone-sensitive |
| 236534_at | 448.35 | 172.08 | 635.59 | 121.54 | 0.71 | BCL2/adenovirus E1B 19 kD interacting protein like |
| 212530_at | 349.47 | 80.11 | 194.88 | 51.02 | 1.79 | NIMA (never in mitosis gene a)-related kinase 7 |
| 233768_at | 952.53 | 169.13 | 416.94 | 122.87 | 2.28 | ATG5 autophagy related 5 homolog (S. cerevisiae) |
| 204589_at | 135.00 | 43.04 | 513.76 | 178.41 | 0.26 | NUAK family, SNF1-like kinase, 1 |
| 208891_at | 311.41 | 105.55 | 650.94 | 120.21 | 0.48 | dual specificity phosphatase 6 |
| 217807_s_at | 373.76 | 104.80 | 1953.53 | 425.79 | 0.19 | glioma tumor suppressor candidate region gene 2 |
| 212099_at | 518.35 | 169.00 | 1379.12 | 221.37 | 0.38 | ras homolog gene family, member B |
| 208634_s_at | 850.29 | 274.14 | 1513.06 | 274.93 | 0.56 | microtubule-actin crosslinking factor 1 |
| 217907_at | 908.18 | 439.70 | 702.94 | 131.03 | 1.29 | mitochondrial ribosomal protein L18 |
| 204351_at | 861.47 | 308.03 | 287.24 | 113.77 | 3.00 | S100 calcium binding protein P |
| 201024_x_at | 634.47 | 259.70 | 226.29 | 59.75 | 2.80 | eukaryotic translation initiation factor 5B |
| 232602_at | 711.29 | 341.22 | 2088.12 | 443.19 | 0.34 | WAP four-disulfide core domain 3 |
| 208691_at | 475.24 | 248.35 | 972.24 | 252.25 | 0.49 | transferrin receptor (p90, CD71); transferrin receptor |
| | | | | | | (p90, CD71) |
| 230791_at | 229.53 | 79.88 | 438.06 | 142.25 | 0.52 | Nuclear factor I/B |
| 217028_at | 2962.06 | 1218.27 | 1139.29 | 497.25 | 2.60 | chemokine (C-X-C motif) receptor 4 |
| 201008_s_at | 382.24 | 259.56 | 1276.82 | 278.04 | 0.30 | thioredoxin interacting protein |
| 207065_at | 742.47 | 248.16 | 1269.00 | 250.38 | 0.59 | cytokeratin type II |
| 217234_s_at | 623.24 | 257.61 | 825.76 | 176.89 | 0.75 | villin 2 (ezrin) |
| 223892_s_at | 359.88 | 148.52 | 634.82 | 150.83 | 0.57 | transmembrane BAX inhibitor motif containing 4 |
| 202575_at | 960.18 | 348.57 | 2012.35 | 426.09 | 0.48 | cellular retinoic acid binding protein 2 |
| 226651_at | 343.29 | 81.39 | 193.41 | 69.77 | 1.77 | homer homolog 1 (Drosophila) |
| 201493_s_at | 237.65 | 77.10 | 491.47 | 115.44 | 0.48 | pumilio homolog 2 (Drosophila) |
| 209351_at | 1426.88 | 873.32 | 3630.82 | 884.25 | 0.39 | keratin 14 (epidermolysis bullosa simplex, Dowling- |
| | | | | | | Meara, Koebner) |
| 237732_at | 573.00 | 266.33 | 646.18 | 133.11 | 0.89 | Transcribed locus, moderately similar to |
| | | | | | | NP_780633.1 hypothetical protein LOC109314 [Mus |
| | | | | | | musculus] |
| 209234_at | 384.47 | 172.19 | 850.29 | 149.65 | 0.45 | kinesin family member 1B |
| 225769_at | 191.24 | 77.19 | 111.71 | 47.79 | 1.71 | component of oligomeric golgi complex 6 |
| 238021_s_at | 170.06 | 49.49 | 660.94 | 144.23 | 0.26 | hypothetical gene supported by AF275804 |
| 206318_at | 195.47 | 44.79 | 81.00 | 11.76 | 2.41 | serine peptidase inhibitor-like, with Kunitz and WAP |
| | | | | | | domains 1 (eppin) |
| 223218_s_at | 323.00 | 87.74 | 164.65 | 51.72 | 1.96 | nuclear factor of kappa light polypeptide gene |
| | | | | | | enhancer in B-cells inhibitor, zeta |
| 201300_s_at | 330.47 | 113.58 | 907.53 | 148.39 | 0.36 | prion protein (p27-30) (Creutzfeld-Jakob disease, |
| | | | | | | Gerstmann-Strausler-Scheinker syndrome, fatal |
| | | | | | | familial insomnia) |
| 202258_s_at | 188.29 | 57.47 | 375.24 | 82.60 | 0.50 | phosphonoformate immuno-associated protein 5 |
| 217917_s_at | 369.35 | 118.13 | 560.76 | 86.39 | 0.66 | dynein, cytoplasmic, light polypeptide 2A |
| 209290_s_at | 135.65 | 38.61 | 769.47 | 162.46 | 0.18 | Nuclear factor I/B |
| 207731_at | 473.82 | 130.66 | 312.12 | 120.11 | 1.52 | |
| 1557158_s_at | 269.12 | 131.42 | 401.47 | 82.48 | 0.67 | myeloid/lymphoid or mixed-lineage leukemia 3 |
| 204268_at | 1071.47 | 756.15 | 1252.29 | 444.30 | 0.86 | S100 calcium binding protein A2 |
| 204734_at | 752.88 | 346.50 | 2514.18 | 520.00 | 0.30 | keratin 15 |
| 209925_at | 384.41 | 161.80 | 1047.29 | 303.69 | 0.37 | occludin |
| 209242_at | 339.88 | 90.88 | 77.94 | 21.54 | 4.36 | paternally expressed 3 |
| 201944_at | 1266.29 | 338.54 | 655.65 | 240.20 | 1.93 | hexosaminidase B (beta polypeptide) |
| 227299_at | 223.29 | 94.27 | 309.59 | 75.46 | 0.72 | Cyclin I |
| 224880_at | 198.88 | 87.74 | 422.53 | 84.73 | 0.47 | v-ral simian leukemia viral oncogene homolog A (ras |
| | | | | | | related) |
| 243933_at | 297.65 | 97.56 | 628.12 | 188.01 | 0.47 | Nuclear factor I/B |
| 228575_at | 551.24 | 173.23 | 1770.71 | 344.63 | 0.31 | fibronectin type III domain containing 6 |
| 201227_s_at | 698.00 | 274.90 | 968.65 | 253.63 | 0.72 | NADH dehydrogenase (ubiquinone) 1 beta |
| | | | | | | subcomplex, 8, 19 kDa |
| 224367_at | 439.53 | 166.79 | 914.12 | 201.07 | 0.48 | brain expressed X-linked 2; brain expressed X-linked 2 |
| 211002_s_at | 857.59 | 389.84 | 1520.47 | 274.73 | 0.56 | tripartite motif-containing 29 |
| 201147_s_at | 502.76 | 180.03 | 940.94 | 249.77 | 0.53 | TIMP metallopeptidase inhibitor 3 (Sorsby fundus |
| | | | | | | dystrophy, pseudoinflammatory) |
| 201699_at | 1186.41 | 424.27 | 376.59 | 114.72 | 3.15 | proteasome (prosome, macropain) 26S subunit, |
| | | | | | | ATPase, 6 |
| 212115_at | 288.88 | 101.77 | 641.12 | 106.92 | 0.45 | chromosome 16 open reading frame 34 |
| 202147_s_at | 677.06 | 151.38 | 135.82 | 40.65 | 4.98 | interferon-related developmental regulator 1 |
| 56256_at | 629.00 | 206.35 | 1530.71 | 333.21 | 0.41 | SID1 transmembrane family, member 2 |
| 206385_s_at | 621.71 | 299.63 | 1172.47 | 316.37 | 0.53 | ankyrin 3, node of Ranvier (ankyrin G) |
| 213033_s_at | 183.59 | 61.74 | 515.94 | 130.14 | 0.36 | Nuclear factor I/B |
| 209595_at | 282.94 | 90.54 | 183.12 | 66.47 | 1.55 | general transcription factor 11F, polypeptide 2 30 kDa |
| 234335_s_at | 252.71 | 73.58 | 574.65 | 139.90 | 0.44 | Family with sequence similarity 84, member A |
| 217339_s_at | 852.00 | 323.60 | 117.06 | 33.28 | 7.28 | pre-B-cell colony enhancing factor 1 |
| 1561737_at | 158.00 | 32.22 | 63.35 | 8.44 | 2.49 | |
| 238320_at | 653.29 | 329.62 | 73.12 | 14.43 | 8.93 | trophoblast-derived noncoding RNA |
| 221215_s_at | 474.29 | 220.10 | 1032.71 | 249.22 | 0.46 | Receptor-interacting serine-threonine kinase 4 |
| 208810_at | 925.29 | 208.01 | 150.18 | 59.93 | 6.16 | DnaJ (Hsp40) homolog, subfamily B, member 6 |
| 202593_s_at | 644.71 | 305.38 | 1081.53 | 247.41 | 0.60 | membrane interacting protein of RGS16 |
| 1567214_a_at | 1238.88 | 442.85 | 355.12 | 141.07 | 3.49 | pinnin, desmosome associated protein |
| 202804_at | 180.00 | 75.19 | 260.00 | 41.09 | 0.69 | ATP-binding cassette, sub-family C (CFTR/MRP), |
| | | | | | | member 1 |
| 229483_at | 141.00 | 47.72 | 334.29 | 106.23 | 0.42 | Ubiquitin-conjugating enzyme E2H (UBC8 homolog, |
| | | | | | | yeast) |
| 221768_at | 109.82 | 36.15 | 475.53 | 190.62 | 0.23 | Splicing factor proline/glutamine-rich |
| | | | | | | (polypyrimidine tract binding protein associated) |
| 1557895_at | 258.59 | 56.42 | 83.53 | 37.37 | 3.10 | FLJ35934 protein |
| 224329_s_at | 284.71 | 83.75 | 95.59 | 36.85 | 2.98 | cornifelin; cornifelin |
| 202054_s_at | 467.53 | 280.22 | 1021.88 | 189.91 | 0.46 | aldehyde dehydrogenase 3 family, member A2 |
| 203124_s_at | 304.12 | 136.31 | 844.65 | 199.07 | 0.36 | solute carrier family 11 (proton-coupled divalent |
| | | | | | | metal ion transporters), member 2 |
| 204094_s_at | 342.18 | 89.96 | 118.94 | 34.17 | 2.88 | TSC22 domain family, member 2 |
| 232307_at | 199.94 | 49.69 | 76.59 | 21.33 | 2.61 | Thyroid hormone receptor associated protein 2 |
| 242611_at | 520.41 | 112.05 | 296.65 | 129.80 | 1.75 | Dedicator of cytokinesis 7 |
| 224862_at | 242.35 | 52.23 | 72.53 | 19.50 | 3.34 | Guanine nucleotide binding protein (G protein), q |
| | | | | | | polypeptide |
| 218422_s_at | 123.88 | 20.74 | 87.24 | 20.98 | 1.42 | Chromosome 13 open reading frame 10 |
| 200814_at | 286.41 | 127.44 | 428.47 | 112.33 | 0.67 | proteasome (prosome, macropain) activator subunit 1 |
| | | | | | | (PA28 alpha) |
| 1552685_a_at | 191.35 | 77.88 | 300.18 | 73.21 | 0.64 | grainyhead-like 1 (Drosophila) |
| 215904_at | 83.00 | 21.19 | 694.82 | 363.76 | 0.12 | myeloid/lymphoid or mixed-lineage leukemia |
| | | | | | | (trithorax homolog, Drosophila); translocated to, 4 |
| 203126_at | 219.59 | 79.18 | 461.82 | 118.35 | 0.48 | inositol(myo)-1(or 4)-monophosphatase 2 |
| 204546_at | 301.41 | 128.08 | 706.35 | 233.85 | 0.43 | KIAA0513 |
| 238427_at | 153.12 | 46.70 | 581.82 | 126.88 | 0.26 | GrpE-like 2, mitochondrial (E. coli) |
| 202753_at | 521.82 | 267.11 | 608.24 | 161.60 | 0.86 | proteasome (prosome, macropain) 26S subunit, non- |
| | | | | | | ATPase, 6 |
| 201337_s_at | 176.41 | 88.92 | 325.65 | 83.35 | 0.54 | vesicle-associated membrane protein 3 (cellubrevin) |
| 231211_s_at | 222.53 | 111.42 | 452.65 | 120.50 | 0.49 | hypothetical LOC541469 protein |
| 222378_at | 253.41 | 105.13 | 63.00 | 12.95 | 4.02 | Hypothetical protein FLJ43663 |
| 212398_at | 106.94 | 14.88 | 64.06 | 20.96 | 1.67 | radixin |
| 240949_x_at | 418.94 | 110.66 | 120.76 | 54.53 | 3.47 | |
| 217196_s_at | 244.12 | 150.82 | 322.06 | 114.52 | 0.76 | calmodulin regulated spectrin-associated protein 1- |
| | | | | | | like 1 |
| 235659_at | 101.76 | 37.07 | 191.47 | 32.13 | 0.53 | Transcribed locus |
| 215945_s_at | 136.53 | 42.96 | 295.47 | 73.08 | 0.46 | tripartite motif-containing 2 |
| 240951_at | 234.71 | 106.47 | 524.94 | 126.25 | 0.45 | CDNA FLJ31407 fis, clone NT2NE2000137 |
| 204542_at | 88.18 | 19.51 | 318.24 | 114.87 | 0.28 | ST6 (alpha-N-acetyl-neuraminyl-2,3-beta-galactosyl- |
| | | | | | | 1,3)-N-acetylgalactosaminide alpha-2,6- |
| | | | | | | sialyltransferase 2 |
| 202551_s_at | 92.06 | 42.57 | 314.47 | 88.33 | 0.29 | cysteine rich transmembrane BMP regulator 1 |
| | | | | | | (chordin-like) |
| 201375_s_at | 246.18 | 83.31 | 398.12 | 77.15 | 0.62 | protein phosphatase 2 (formerly 2A), catalytic |
| | | | | | | subunit, beta isoform |
| 221269_s_at | 120.24 | 44.14 | 178.47 | 28.79 | 0.67 | SH3 domain binding glutamic acid-rich protein like 3; |
| | | | | | | SH3 domain binding glutamic acid-rich protein like 3 |
| 223044_at | 185.00 | 69.75 | 755.18 | 246.36 | 0.24 | solute carrier family 40 (iron-regulated transporter), |
| | | | | | | member 1 |
| 220044_x_at | 111.71 | 44.41 | 268.35 | 82.29 | 0.42 | cisplatin resistance-associated overexpressed protein |
| 224563_at | 80.65 | 26.51 | 177.29 | 35.97 | 0.45 | WAS protein family, member 2 |
| 223068_at | 118.12 | 50.59 | 442.65 | 107.70 | 0.27 | echinoderm microtubule associated protein like 4 |
| 1566428_at | 274.65 | 61.29 | 80.71 | 29.92 | 3.40 | |
| 201894_s_at | 161.47 | 79.84 | 335.12 | 84.13 | 0.48 | signal sequence receptor, alpha (translocon- |
| | | | | | | associated protein alpha) |
| 228121_at | 85.76 | 26.70 | 300.29 | 64.93 | 0.29 | Transforming growth factor, beta 2 |
| 234282_at | 250.94 | 59.28 | 66.82 | 16.48 | 3.76 | MRNA; cDNA DKFZp586E1423 (from clone |
| | | | | | | DKFZp586E1423) |
| 243386_at | 125.24 | 42.30 | 569.65 | 178.47 | 0.22 | Similar to cDNA sequence BC035954 |
| 222932_at | 94.71 | 28.31 | 212.65 | 50.00 | 0.45 | ets homologous factor |
| 204400_at | 591.88 | 248.60 | 1112.59 | 269.60 | 0.53 | embryonal Fyn-associated substrate |
| 209323_at | 474.76 | 144.86 | 136.35 | 63.40 | 3.48 | protein-kinase, interferon-inducible double stranded |
| | | | | | | RNA dependent inhibitor, repressor of (P58 |
| | | | | | | repressor) |
| 200631_s_at | 148.41 | 72.92 | 318.12 | 86.03 | 0.47 | SET translocation (myeloid leukemia-associated) |
| 202376_at | 315.82 | 187.71 | 375.59 | 72.30 | 0.84 | serpin peptidase inhibitor, clade A (alpha-1 |
| | | | | | | antiproteinase, antitrypsin), member 3 |
| 222390_at | 149.71 | 60.95 | 277.18 | 67.12 | 0.54 | WW domain containing adaptor with coiled-coil |
| 1555326_a_at | 514.35 | 186.38 | 236.41 | 89.71 | 2.18 | ADAM metallopeptidase domain 9 (meltrin gamma) |
| 202436_s_at | 800.24 | 499.39 | 2535.06 | 577.13 | 0.32 | cytochrome P450, family 1, subfamily B, polypeptide 1 |
| 1553081_at | 1783.59 | 727.52 | 206.41 | 96.40 | 8.64 | WAP four-disulfide core domain 12 |
| 200602_at | 221.29 | 67.83 | 856.41 | 213.85 | 0.26 | amyloid beta (A4) precursor protein (peptidase nexin- |
| | | | | | | II, Alzheimer disease) |
| 210718_s_at | 567.24 | 98.69 | 169.71 | 59.67 | 3.34 | ADP-ribosylation factor-like |
| 226765_at | 128.47 | 69.92 | 349.06 | 69.88 | 0.37 | Spectrin, beta, non-erythrocytic 1 |
| 208986_at | 294.82 | 151.72 | 830.47 | 168.77 | 0.36 | transcription factor 12 (HTF4, helix-loop-helix |
| | | | | | | transcription factors 4) |
| 209626_s_at | 144.29 | 77.56 | 332.94 | 98.24 | 0.43 | oxysterol binding protein-like 3 |
| 202769_at | 204.00 | 72.32 | 60.35 | 12.65 | 3.38 | Cyclin G2 |
| 202504_at | 209.41 | 145.00 | 480.53 | 108.24 | 0.44 | tripartite motif-containing 29 |
| 233641_s_at | 180.76 | 73.38 | 910.94 | 269.83 | 0.20 | Chromosome 8 open reading frame 13 |
| 1564333_a_at | 326.00 | 148.69 | 535.29 | 145.06 | 0.61 | Sortilin-related VPS10 domain containing receptor 2 |
| 203076_s_at | 348.29 | 122.51 | 1373.00 | 374.00 | 0.25 | SMAD, mothers against DPP homolog 2 (Drosophila) |
| 201232_s_at | 105.18 | 31.14 | 199.47 | 41.09 | 0.53 | proteasome (prosome, macropain) 26S subunit, non- |
| | | | | | | ATPase, 13 |
| 224595_at | 244.00 | 105.81 | 543.76 | 147.40 | 0.45 | solute carrier family 44, member 1 |
| 243902_at | 204.88 | 43.60 | 69.88 | 20.48 | 2.93 | |
| 225450_at | 210.29 | 109.33 | 462.00 | 138.96 | 0.46 | angiomotin like 1 |
| 232361_s_at | 125.82 | 56.44 | 539.71 | 193.11 | 0.23 | ets homologous factor |
| 207356_at | 2836.18 | 1038.70 | 60.88 | 30.86 | 46.58 | defensin, beta 4 |
| 201286_at | 188.53 | 108.84 | 768.65 | 146.91 | 0.25 | syndecan 1 |
| 237030_at | 256.82 | 113.59 | 705.88 | 208.16 | 0.36 | acid phosphatase, prostate |
| 227095_at | 121.94 | 52.31 | 464.41 | 119.81 | 0.26 | Leptin receptor |
| 209091_s_at | 192.53 | 66.88 | 789.71 | 312.70 | 0.24 | SH3-domain GRB2-like endophilin B1 |
| 208073_x_at | 127.76 | 46.24 | 406.35 | 141.11 | 0.31 | tetratricopeptide repeat domain 3 |
| 224893_at | 189.71 | 82.23 | 442.00 | 160.00 | 0.43 | DKFZP564J0863 protein |
| 212307_s_at | 85.71 | 24.51 | 216.06 | 56.64 | 0.40 | O-linked N-acetylglucosamine (GlcNAc) transferase |
| | | | | | | (UDP-N-acetylglucosamine: polypeptide-N- |
| | | | | | | acetylglucosaminyl transferase) |
| 212774_at | 244.18 | 60.48 | 96.88 | 50.66 | 2.52 | zinc finger protein 238 |
| 237625_s_at | 315.35 | 99.85 | 63.29 | 26.62 | 4.98 | Immunoglobulin kappa variable 1-5 |
| 227638_at | 383.53 | 113.84 | 103.88 | 32.84 | 3.69 | KIAA1632 |
| 231697_s_at | 559.29 | 194.54 | 166.24 | 56.93 | 3.36 | Transmembrane protein 49 |
| 225060_at | 132.35 | 46.36 | 288.24 | 78.95 | 0.46 | low density lipoprotein receptor-related protein 11 |
| 224754_at | 175.82 | 87.48 | 311.24 | 111.83 | 0.56 | Sp1 transcription factor |
| 1570361_a_at | 195.18 | 51.56 | 48.00 | 13.89 | 4.07 | Homo sapiens, clone IMAGE: 3935253, mRNA |
| 40189_at | 113.06 | 55.12 | 215.59 | 61.86 | 0.52 | SET translocation (myeloid leukemia-associated) |
| 213029_at | 88.35 | 32.42 | 509.65 | 121.55 | 0.17 | Nuclear factor I/B |
| 240502_at | 363.29 | 98.65 | 63.35 | 17.86 | 5.73 | FERM domain containing 5 |
| 201820_at | 609.12 | 442.53 | 1796.00 | 584.15 | 0.34 | keratin 5 (epidermolysis bullosa simplex, Dowling- |
| | | | | | | Meara/Kobner/Weber-Cockayne types) |
| 1560558_at | 254.47 | 69.02 | 134.18 | 66.51 | 1.90 | chromosome 9 open reading frame 80 |
| 227036_at | 49.59 | 18.70 | 129.35 | 27.11 | 0.38 | Transcribed locus |
| 228496_s_at | 95.29 | 33.22 | 308.76 | 93.69 | 0.31 | Cysteine rich transmembrane BMP regulator 1 |
| | | | | | | (chordin-like) |
| 218340_s_at | 185.41 | 59.69 | 48.53 | 15.63 | 3.82 | hypothetical protein FLJ10808 |
| 222404_x_at | 83.29 | 43.33 | 578.82 | 106.48 | 0.14 | butyrate-induced transcript 1 |
| 228613_at | 152.94 | 34.48 | 54.35 | 13.05 | 2.81 | RAB11 family interacting protein 3 (class II) |
| 209681_at | 148.18 | 63.53 | 56.35 | 19.56 | 2.63 | solute carrier family 19 (thiamine transporter), |
| | | | | | | member 2 |
| 1553412_at | 404.71 | 102.97 | 70.41 | 25.98 | 5.75 | ATP/GTP binding protein-like 4 |
| 202814_s_at | 182.47 | 47.66 | 94.94 | 27.03 | 1.92 | hexamethylene bis-acetamide inducible 1 |
| 201266_at | 294.71 | 104.08 | 55.53 | 18.81 | 5.31 | thioredoxin reductase 1 |
| 231061_at | 248.94 | 105.76 | 82.06 | 31.25 | 3.03 | HDCMA18P protein |
| 209286_at | 104.59 | 32.53 | 92.47 | 46.19 | 1.13 | CDC42 effector protein (Rho GTPase binding) 3 |
| 228332_s_at | 246.06 | 112.33 | 482.76 | 100.15 | 0.51 | chromosome 11 open reading frame 31 |
| 234000_s_at | 43.12 | 22.80 | 269.71 | 56.92 | 0.16 | butyrate-induced transcript 1 |
| 218309_at | 203.35 | 64.05 | 102.53 | 48.83 | 1.98 | calcium/calmodulin-dependent protein kinase II |
| | | | | | | inhibitor 1 |
| 225538_at | 333.41 | 103.98 | 94.71 | 40.82 | 3.52 | zinc finger, CCHC domain containing 9 |
| 202687_s_at | 122.29 | 60.53 | 231.94 | 76.30 | 0.53 | tumor necrosis factor (ligand) superfamily, member |
| | | | | | | 10; tumor necrosis factor (ligand) superfamily, |
| | | | | | | member 10 |
| 1564358_at | 251.24 | 49.67 | 79.65 | 38.30 | 3.15 | CDNA: FLJ22631 fis, clone HSI06451 |
| 202449_s_at | 124.82 | 52.61 | 236.82 | 64.35 | 0.53 | retinoid X receptor, alpha |
| 200607_s_at | 102.59 | 47.53 | 167.47 | 51.08 | 0.61 | RAD21 homolog (S. pombe) |
| 241930_x_at | 99.35 | 24.48 | 37.47 | 10.31 | 2.65 | |
| 230788_at | 115.59 | 65.03 | 996.59 | 359.74 | 0.12 | glucosaminyl (N-acetyl) transferase 2, 1-branching |
| | | | | | | enzyme |
| 1569906_s_at | 412.47 | 129.30 | 137.12 | 72.92 | 3.01 | PHD finger protein 20 |
| 206643_at | 988.65 | 282.37 | 54.71 | 28.54 | 18.07 | histidine ammonia-lyase |
| 224806_at | 222.24 | 56.32 | 116.06 | 51.52 | 1.91 | tripartite motif-containing 25 |
| 217756_x_at | 62.24 | 25.51 | 115.12 | 27.68 | 0.54 | small EDRK-rich factor 2 |
| 220974_x_at | 119.88 | 63.74 | 398.82 | 126.98 | 0.30 | sideroflexin 3; sideroflexin 3 |
| 214581_x_at | 94.65 | 38.88 | 574.12 | 201.07 | 0.16 | tumor necrosis factor receptor superfamily, member |
| | | | | | | 21 |
| 218816_at | 47.88 | 20.33 | 310.71 | 92.00 | 0.15 | leucine rich repeat containing 1 |
| 1570227_at | 152.24 | 36.24 | 52.71 | 15.99 | 2.89 | Chromosome 16 open reading frame 49 |
| 201163_s_at | 129.71 | 31.36 | 72.12 | 26.53 | 1.80 | insulin-like growth factor binding protein 7 |
| 240503_at | 138.41 | 29.63 | 48.71 | 21.40 | 2.84 | R3H domain containing 1 |
| 226254_s_at | 50.00 | 21.26 | 244.88 | 96.77 | 0.20 | KIAA1430 |
| 206011_at | 79.59 | 34.40 | 305.12 | 98.36 | 0.26 | caspase 1, apoptosis-related cysteine peptidase |
| | | | | | | (interleukin 1, beta, convertase) |
| 1561155_at | 269.24 | 61.51 | 59.18 | 42.90 | 4.55 | Cation channel, sperm associated 2 |
| 212973_at | 55.41 | 11.86 | 127.35 | 26.49 | 0.44 | Ribose 5-phosphate isomerase A (ribose 5-phosphate |
| | | | | | | epimerase) |
| 216591_s_at | 247.24 | 93.77 | 97.53 | 51.43 | 2.53 | succinate dehydrogenase complex, subunit C, integral |
| | | | | | | membrane protein, 15 kDa |
| 226257_x_at | 38.12 | 13.44 | 107.76 | 31.86 | 0.35 | mitochondrial ribosomal protein S22 |
| 223598_at | 74.35 | 39.31 | 360.65 | 92.86 | 0.21 | RAD23 homolog B (S. cerevisiae) |
| 201130_s_at | 76.18 | 38.57 | 284.47 | 95.34 | 0.27 | cadherin 1, type 1, E-cadherin (epithelial) |
| 238165_at | 82.94 | 42.82 | 540.53 | 250.20 | 0.15 | PDZ domain containing RING finger 3 |
| 201150_s_at | 90.47 | 54.14 | 225.82 | 51.68 | 0.40 | TIMP metallopeptidase inhibitor 3 (Sorsby fundus |
| | | | | | | dystrophy, pseudoinflammatory) |
| 230180_at | 66.35 | 25.15 | 169.35 | 55.85 | 0.39 | DEAD (Asp-Glu-Ala-Asp) box polypeptide 17 |
| 231662_at | 644.00 | 271.08 | 26.06 | 7.85 | 24.71 | Arginase, liver |
| 202859_x_at | 911.88 | 511.74 | 37.76 | 13.09 | 24.15 | interleukin 8 |
| 1569476_at | 159.18 | 32.07 | 52.59 | 11.73 | 3.03 | DKFZP434L187 protein |
| 227728_at | 132.82 | 67.17 | 262.53 | 79.22 | 0.51 | Protein phosphatase 1A (formerly 2C), magnesium- |
| | | | | | | dependent, alpha isoform |
| 202724_s_at | 164.24 | 63.43 | 282.59 | 75.09 | 0.58 | forkhead box O1A (rhabdomyosarcoma) |
| 225117_at | 66.35 | 30.53 | 226.24 | 45.76 | 0.29 | LOC284058 protein |
| 200776_s_at | 351.76 | 128.94 | 93.59 | 40.81 | 3.76 | basic leucine zipper and W2 domains 1; similar to |
| | | | | | | basic leucine zipper and W2 domains 1 |
| 226018_at | 61.47 | 26.72 | 313.88 | 150.56 | 0.20 | hypothetical protein Ells1 |
| 240783_at | 168.65 | 43.27 | 33.00 | 12.37 | 5.11 | Transcribed locus |
| 229004_at | 57.35 | 29.79 | 390.71 | 93.30 | 0.15 | ADAM metallopeptidase with thrombospondin type 1 |
| | | | | | | motif, 15 |
| 225100_at | 803.06 | 229.58 | 79.35 | 30.18 | 10.12 | F-box protein 45 |
| 231152_at | 240.59 | 106.72 | 61.53 | 23.99 | 3.91 | MRNA; cDNA DKFZp686D22106 (from clone |
| | | | | | | DKFZp686D22106) |
| 209719_x_at | 1451.24 | 540.95 | 20.59 | 11.08 | 70.49 | serpin peptidase inhibitor, clade B (ovalbumin), |
| | | | | | | member 3 |
| 239860_at | 675.24 | 211.89 | 192.53 | 79.26 | 3.51 | Hypothetical protein PRO2949 |
| 201433_s_at | 133.76 | 73.89 | 235.53 | 73.57 | 0.57 | phosphatidylserine synthase 1 |
| 226029_at | 71.88 | 33.43 | 220.06 | 51.37 | 0.33 | vang-like 2 (van gogh, Drosophila) |
| 212071_s_at | 85.00 | 51.63 | 204.65 | 55.28 | 0.42 | spectrin, beta, non-erythrocytic 1 |
| 224569_s_at | 68.24 | 17.08 | 247.53 | 103.62 | 0.28 | Interferon regulatory factor 2 binding protein 2 |
| 210186_s_at | 102.76 | 52.93 | 401.71 | 128.01 | 0.26 | FK506 binding protein 1A, 12 kDa |
| 215424_s_at | 103.65 | 46.29 | 386.12 | 127.77 | 0.27 | SNW domain containing 1 |
| 233519_at | 106.71 | 16.25 | 57.24 | 14.04 | 1.86 | ADP-ribosylation factor-like |
| 238386_x_at | 176.00 | 44.61 | 67.71 | 18.72 | 2.60 | Transcribed locus |
| 222242_s_at | 156.47 | 62.50 | 539.59 | 112.57 | 0.29 | kallikrein 5 |
| 223716_s_at | 124.59 | 45.49 | 316.94 | 72.03 | 0.39 | zinc finger protein 265 |
| 208674_x_at | 81.29 | 27.01 | 153.06 | 54.67 | 0.53 | glycosyltransferase |
| 1560587_s_at | 207.76 | 104.59 | 505.29 | 1360.50 | 0.41 | peroxiredoxin 5 |
| 210020_x_at | 111.59 | 40.56 | 279.35 | 71.05 | 0.40 | calmodulin-like 3 |
| 212915_at | 85.82 | 37.38 | 228.12 | 57.54 | 0.38 | PDZ domain containing RING finger 3 |
| 233879_at | 208.82 | 53.29 | 92.82 | 43.83 | 2.25 | TPTE and PTEN homologous inositol lipid |
| | | | | | | phosphatase pseudogene |
| 212664_at | 133.24 | 30.13 | 64.12 | 19.16 | 2.08 | tubulin, beta 4 |
| 226508_at | 163.24 | 64.46 | 73.18 | 26.89 | 2.23 | polyhomeotic like 3 (Drosophila) |
| 212276_at | 105.29 | 36.32 | 511.06 | 123.99 | 0.21 | lipin 1 |
| 221488_s_at | 241.41 | 123.56 | 465.76 | 116.73 | 0.52 | Chromosome 6 open reading frame 82 |
| 1559492_at | 892.59 | 294.42 | 63.53 | 29.95 | 14.05 | CDNA clone IMAGE: 5268696 |
| 1569200_at | 205.00 | 55.25 | 53.76 | 12.28 | 3.81 | SEC15-like 2 (S. cerevisiae) |
| 1553613_s_at | 29.24 | 9.39 | 243.00 | 53.97 | 0.12 | forkhead box C1 |
| 223449_at | 46.00 | 12.67 | 215.53 | 56.29 | 0.21 | sema domain, transmembrane domain (TM), and |
| | | | | | | cytoplasmic domain, (semaphorin) 6A |
| 217897_at | 248.88 | 184.98 | 402.29 | 129.54 | 0.62 | FXYD domain containing ion transport regulator 6 |
| 1553749_at | 80.82 | 32.66 | 166.94 | 32.39 | 0.48 | family with sequence similarity 76, member B |
| 200862_at | 67.06 | 22.88 | 142.88 | 29.15 | 0.47 | 24-dehydrocholesterol reductase |
| 222431_at | 80.88 | 22.16 | 248.35 | 74.96 | 0.33 | spindlin |
| 220184_at | 252.76 | 61.01 | 113.29 | 47.12 | 2.23 | Nanog homeobox |
| 37152_at | 707.71 | 239.87 | 215.65 | 93.27 | 3.28 | peroxisome proliferative activated receptor, delta |
| 209240_at | 52.94 | 21.46 | 130.59 | 37.98 | 0.41 | O-linked N-acetylglucosamine (GlcNAc) transferase |
| | | | | | | (UDP-N-acetylglucosamine: polypeptide-N- |
| | | | | | | acetylglucosaminyl transferase) |
| 1557717_at | 213.41 | 65.50 | 62.00 | 18.86 | 3.44 | hypothetical protein LOC338862 |
| 224899_s_at | 88.53 | 56.41 | 162.47 | 43.63 | 0.54 | implantation-associated protein |
| 208313_s_at | 108.18 | 52.11 | 295.47 | 120.63 | 0.37 | splicing factor 1 |
| 212416_at | 94.12 | 56.67 | 274.00 | 82.75 | 0.34 | secretory carrier membrane protein 1 |
| 226563_at | 65.94 | 25.42 | 226.06 | 72.91 | 0.29 | SMAD, mothers against DPP homolog 2 (Drosophila) |
| 1561720_at | 238.71 | 76.34 | 76.29 | 57.66 | 3.13 | RecQ protein-like 5 |
| 208023_at | 237.71 | 66.67 | 53.00 | 27.03 | 4.49 | tumor necrosis factor receptor superfamily, member 4 |
| 212781_at | 306.76 | 84.32 | 107.65 | 41.37 | 2.85 | retinoblastoma binding protein 6 |
| 203705_s_at | 184.06 | 121.50 | 538.00 | 174.99 | 0.34 | frizzled homolog 7 (Drosophila) |
| 215936_s_at | 143.41 | 30.41 | 27.41 | 10.38 | 5.23 | KIAA1033 |
| 1557433_at | 98.76 | 21.92 | 35.29 | 16.44 | 2.80 | |
| 238669_at | 53.12 | 10.73 | 168.29 | 45.96 | 0.32 | prostaglandin-endoperoxide synthase 1 (prostaglandin |
| | | | | | | G/H synthase and cyclooxygenase) |
| 1556021_at | 127.06 | 30.90 | 42.59 | 20.80 | 2.98 | hypothetical protein LOC144874 |
| 203263_s_at | 66.88 | 35.42 | 223.12 | 60.90 | 0.30 | Cdc42 guanine nucleotide exchange factor (GEF) 9 |
| 201661_s_at | 59.59 | 23.35 | 214.94 | 81.46 | 0.28 | acyl-CoA synthetase long-chain family member 3 |
| 239719_at | 81.76 | 47.24 | 213.76 | 80.66 | 0.38 | CD109 antigen (Gov platelet alloantigens) |
| 231775_at | 127.76 | 31.21 | 44.82 | 18.82 | 2.85 | tumor necrosis factor receptor superfamily, member |
| | | | | | | 10a |
| 209590_at | 94.59 | 48.35 | 158.76 | 46.97 | 0.60 | Bone morphogenetic protein 7 (osteogenic protein 1) |
| 235556_at | 28.41 | 14.97 | 376.06 | 144.87 | 0.08 | Transcribed locus, weakly similar to NP_703324.1 |
| | | | | | | glutamic acid-rich protein (garp) [Plasmodium |
| | | | | | | falciparum 3D7] |
| 212352_s_at | 125.76 | 92.00 | 261.35 | 82.79 | 0.48 | transmembrane emp24-like trafficking protein 10 |
| | | | | | | (yeast) |
| 201287_s_at | 118.06 | 65.10 | 287.41 | 78.54 | 0.41 | syndecan 1 |
| 202696_at | 251.71 | 119.06 | 55.59 | 15.93 | 4.53 | oxidative-stress responsive 1 |
| 229115_at | 114.76 | 57.06 | 343.94 | 117.48 | 0.33 | dynein, cytoplasmic, heavy polypeptide 1 |
| 226960_at | 182.47 | 53.44 | 41.88 | 6.99 | 4.36 | DMC |
| 211466_at | 92.24 | 43.80 | 217.06 | 63.70 | 0.42 | nuclear factor I/B |
| 226614_s_at | 145.71 | 84.37 | 303.24 | 92.55 | 0.48 | chromosome 8 open reading frame 13 |
| 212077_at | 29.00 | 16.96 | 409.76 | 125.45 | 0.07 | caldesmon 1 |
| 209369_at | 192.41 | 125.99 | 495.06 | 193.88 | 0.39 | annexin A3 |
| 212904_at | 14.94 | 3.58 | 138.24 | 31.22 | 0.11 | leucine rich repeat containing 47 |
| 215150_at | 196.12 | 63.29 | 24.47 | 5.41 | 8.01 | YOD1 OTU deubiquinating enzyme 1 homolog |
| | | | | | | (yeast) |
| 202102_s_at | 49.94 | 16.75 | 188.82 | 53.16 | 0.26 | bromodomain containing 4 |
| 225782_at | 65.82 | 49.79 | 438.59 | 169.79 | 0.15 | methionine sulfoxide reductase B3 |
| 201085_s_at | 56.12 | 27.50 | 150.06 | 38.81 | 0.37 | SON DNA binding protein |
| 202912_at | 215.14 | 85.09 | 52.12 | 10.50 | 4.13 | adrenomedullin |
| 244087_at | 179.59 | 58.24 | 47.59 | 8.94 | 3.77 | Adenosine monophosphate deaminase (isoform E) |
| 209365_s_at | 228.41 | 80.63 | 47.18 | 20.65 | 4.84 | extracellular matrix protein 1 |
| 200824_at | 80.59 | 35.83 | 136.35 | 45.18 | 0.59 | glutathione S-transferase pi |
| 1570593_at | 188.24 | 61.39 | 70.94 | 36.63 | 2.65 | ATPase, aminophospholipid transporter (APLT), |
| | | | | | | Class I, type 8A, member 1 |
| 205763_s_at | 131.00 | 60.66 | 36.24 | 19.20 | 3.62 | DEAD (Asp-Glu-Ala-Asp) box polypeptide 18 |
| 210596_at | 31.47 | 10.95 | 163.06 | 43.62 | 0.19 | |
| 1554996_at | 214.65 | 59.57 | 36.29 | 12.94 | 5.91 | zinc finger protein 479 |
| 211383_s_at | 46.65 | 4.32 | 148.53 | 32.92 | 0.31 | WD repeat domain 37 |
| 238856_s_at | 129.35 | 53.45 | 37.35 | 8.58 | 3.46 | Pantothenate kinase 2 (Hallervorden-Spatz syndrome) |
| 219735_s_at | 97.00 | 78.27 | 185.65 | 65.87 | 0.52 | transcription factor CP2-like 1 |
| 226343_at | 45.88 | 15.29 | 133.00 | 37.73 | 0.34 | Dipeptidylpeptidase 8 |
| 1569909_at | 98.47 | 65.72 | 202.18 | 64.62 | 0.49 | keratin 6L |
| 201149_s_at | 84.94 | 42.31 | 351.65 | 155.48 | 0.24 | TIMP metallopeptidase inhibitor 3 (Sorsby fundus |
| | | | | | | dystrophy, pseudoinflammatory) |
| 240926_at | 120.94 | 27.82 | 76.82 | 53.33 | 1.57 | leucine-rich repeats and calponin homology (CH) |
| | | | | | | domain containing 3 |
| 206591_at | 141.41 | 32.38 | 37.71 | 10.82 | 4.46 | recombination activating gene 1 |
| 221854_at | 50.24 | 8.47 | 126.76 | 34.74 | 0.40 | plakophilin 1 (ectodermal dysplasia/skin fragility |
| | | | | | | syndrome) |
| 244770_at | 289.82 | 106.20 | 161.71 | 101.54 | 1.79 | Hypothetical gene supported by AK091718 |
| 202592_at | 42.88 | 8.16 | 145.29 | 52.46 | 0.30 | biogenesis of lysosome-related organelles complex-1, |
| | | | | | | subunit 1 |
| 243960_x_at | 114.82 | 28.28 | 88.35 | 39.68 | 1.30 | DKFZp434A0131 protein |
| 214446_at | 263.94 | 110.91 | 66.18 | 27.57 | 3.99 | elongation factor, RNA polymerase II, 2 |
| 1567213_at | 183.76 | 79.43 | 43.24 | 20.75 | 4.25 | pinin, desmosome associated protein |
| 215513_at | 108.71 | 29.48 | 31.35 | 16.47 | 3.47 | hydatidiform mole associated and imprinted |
| 241786_at | 81.35 | 21.52 | 49.35 | 28.15 | 1.65 | Protein phosphatase 3 (formerly 2B), regulatory |
| | | | | | | subunit B, 19 kDa, alpha isoform (calcineurin B, type |
| | | | | | | I) |
| 213136_at | 238.12 | 110.65 | 51.65 | 21.69 | 4.61 | protein tyrosine phosphatase, non-receptor type 2 |
| 201465_s_at | 34.12 | 6.71 | 78.00 | 12.75 | 0.44 | v-jun sarcoma virus 17 oncogene homolog (avian) |
| 204832_s_at | 153.18 | 52.79 | 52.82 | 22.73 | 2.90 | bone morphogenetic protein receptor, type IA |
| 204029_at | 68.71 | 31.13 | 185.41 | 52.15 | 0.37 | cadherin, EGF LAG seven-pass G-type receptor |
| | | | | | | (flamingo homolog, Drosophila) |
| 219864_s_at | 47.18 | 15.52 | 293.76 | 82.68 | 0.16 | Down syndrome critical region gene 1-like 2 |
| 222249_at | 95.53 | 23.16 | 32.41 | 6.73 | 3.04 | KIAA1651 protein |
| 232281_at | 231.79 | 67.05 | 53.00 | 15.07 | 4.37 | CDNA FLJ12237 fis, clone MAMMA1001249 |
| 204818_at | 228.47 | 184.09 | 567.94 | 160.76 | 0.40 | hydroxysteroid (17-beta) dehydrogenase 2 |
| 220128_s_at | 95.94 | 48.88 | 158.41 | 39.54 | 0.61 | NIPA-like domain containing 2 |
| 1554912_at | 433.59 | 131.26 | 62.82 | 34.59 | 6.90 | family with sequence similarity 62 (C2 domain |
| | | | | | | containing), member C |
| 234522_at | 182.88 | 50.65 | 21.06 | 4.56 | 8.68 | Similar to KIAA0160 gene product is novel |
| 232500_at | 114.94 | 26.92 | 23.06 | 9.16 | 4.98 | chromosome 20 open reading frame 74 |
| 213322_at | 47.76 | 24.22 | 115.65 | 40.34 | 0.41 | chromosome 6 open reading frame 130 |
| 218974_at | 171.47 | 41.94 | 61.88 | 52.49 | 2.77 | hypothetical protein FLJ10159 |
| 222473_s_at | 266.47 | 97.67 | 91.29 | 38.58 | 2.92 | erbb2 interacting protein |
| 242688_at | 133.35 | 34.41 | 33.88 | 4.89 | 3.94 | Thyroid hormone receptor interactor 12 |
| 213701_at | 22.65 | 4.84 | 121.24 | 42.08 | 0.19 | hypothetical protein DKFZp434N2030 |
| 221515_s_at | 29.71 | 14.14 | 114.82 | 32.99 | 0.26 | leucine carboxyl methyltransferase 1 |
| 232170_at | 498.88 | 251.27 | 46.41 | 14.26 | 10.75 | S100 calcium binding protein A7-like 1 |
| 1559890_a_at | 56.88 | 39.24 | 186.88 | 65.65 | 0.30 | abl-interactor 1 |
| 244016_at | 233.24 | 88.72 | 49.29 | 21.38 | 4.73 | Transcribed locus |
| 206177_s_at | 486.94 | 222.17 | 84.59 | 60.23 | 5.76 | arginase, liver |
| 222484_s_at | 485.88 | 345.96 | 1637.76 | 657.02 | 0.30 | chemokine (C-X-C motif) ligand 14 |
| 225694_at | 88.94 | 53.03 | 224.35 | 95.68 | 0.40 | CDC2-related protein kinase 7 |
| 203177_x_at | 153.00 | 44.80 | 126.41 | 74.66 | 1.21 | transcription factor A, mitochondrial |
| 219388_at | 59.82 | 29.20 | 99.76 | 29.24 | 0.60 | grainyhead-like 2 (Drosophila) |
| 218076_s_at | 78.12 | 62.01 | 156.06 | 47.05 | 0.50 | Rho GTPase activating protein 17 |
| 244826_at | 122.82 | 44.17 | 31.65 | 13.34 | 3.88 | Phosphatidylinositol transfer protein, beta |
| 220197_at | 48.12 | 32.35 | 156.29 | 41.83 | 0.31 | ATPase, H+ transporting, lysosomal V0 subunit a |
| | | | | | | isoform 4 |
| 231969_at | 117.82 | 42.14 | 34.24 | 11.41 | 3.44 | storkhead box 2 |
| 239321_at | 27.82 | 7.97 | 225.47 | 87.76 | 0.12 | Hypothetical gene supported by BC013859 |
| 216414_at | 124.65 | 27.43 | 42.82 | 17.67 | 2.91 | |
| 209688_s_at | 58.24 | 29.90 | 176.53 | 59.42 | 0.33 | hypothetical protein FLJ10996 |
| 218310_at | 196.18 | 79.46 | 53.41 | 21.32 | 3.67 | RAB guanine nucleotide exchange factor (GEF) 1 |
| 215253_s_at | 53.47 | 35.63 | 233.88 | 83.47 | 0.23 | Down syndrome critical region gene 1 |
| 210544_s_at | 57.53 | 32.73 | 238.59 | 71.04 | 0.24 | aldehyde dehydrogenase 3 family, member A2 |
| 238006_at | 80.82 | 22.15 | 37.12 | 7.79 | 2.18 | SIN3 homolog A, transcription regulator (yeast) |
| 1554660_a_at | 320.76 | 89.61 | 100.82 | 52.15 | 3.18 | chromosome 1 open reading frame 71 |
| 1561343_a_at | 149.65 | 41.98 | 51.35 | 26.84 | 2.91 | hypothetical protein LOC150005 |
| 233781_s_at | 102.82 | 34.00 | 25.35 | 5.81 | 4.06 | Nebulin |
| 200955_at | 48.47 | 22.90 | 164.53 | 51.00 | 0.29 | inner membrane protein, mitochondrial (mitofilin) |
| 218740_s_at | 49.71 | 35.90 | 232.71 | 89.38 | 0.21 | CDK5 regulatory subunit associated protein 3 |
| 211063_s_at | 50.41 | 16.97 | 115.88 | 37.36 | 0.44 | NCK adaptor protein 1; NCK adaptor protein 1 |
| 211708_s_at | 115.12 | 74.56 | 189.71 | 71.374 | 0.61 | stearoyl-CoA desaturase (delta-9-desaturase); |
| | | | | | | stearoyl-CoA desaturase (delta-9-desaturase) |
| 41469_at | 380.53 | 125.71 | 18.82 | 5.77 | 20.22 | peptidase inhibitor 3, skin-derived (SKALP) |
| 1563660_at | 124.76 | 28.82 | 39.53 | 10.74 | 3.16 | MRNA; cDNA DKFZp686A0837 (from clone |
| | | | | | | DKFZp686A0837) |
| 221011_s_at | 38.65 | 21.76 | 81.47 | 19.10 | 0.47 | likely ortholog of mouse limb-bud and heart gene; |
| | | | | | | likely ortholog of mouse limb-bud and heart gene |
| 217738_at | 180.53 | 61.32 | 71.53 | 41.06 | 2.52 | pre-B-cell colony enhancing factor 1 |
| 201244_s_at | 216.59 | 74.67 | 64.12 | 29.67 | 3.38 | v-raf-1 murine leukemia viral oncogene homolog 1 |
| 217226_s_at | 41.35 | 21.21 | 136.88 | 46.26 | 0.30 | sideroflexin 3 |
| 222230_s_at | 34.82 | 15.99 | 135.71 | 46.83 | 0.26 | actin-related protein 10 homolog (S. cerevisiae) |
| 228926_s_at | 38.88 | 21.54 | 112.06 | 32.19 | 0.35 | SWI/SNF related, matrix associated, actin dependent |
| | | | | | | regulator of chromatin, subfamily a, member 2 |
| 238041_at | 28.59 | 14.87 | 149.00 | 49.43 | 0.19 | Transcription factor 12 (HTF4, helix-loop-helix |
| | | | | | | transcription factors 4) |
| 214388_at | 25.47 | 5.49 | 165.00 | 78.32 | 0.15 | Hypothetical gene CG012 |
| 234784_at | 62.06 | 22.84 | 25.59 | 10.54 | 2.43 | chromosome 11 open reading frame 41 |
| 1552946_at | 108.65 | 42.45 | 20.06 | 8.90 | 5.42 | zinc finger protein 114 |
| 224067_at | 60.18 | 14.07 | 24.47 | 7.83 | 2.46 | |
| 218706_s_at | 149.41 | 62.61 | 26.76 | 6.43 | 5.58 | HCV NS3-transactivated protein 2 |
| 240901_at | 255.65 | 91.41 | 59.82 | 26.44 | 4.27 | Transcribed locus |
| 238480_at | 22.76 | 9.94 | 77.82 | 19.86 | 0.29 | Chromosome 18 open reading frame 50 |
| 1558733_at | 41.47 | 12.25 | 191.71 | 63.96 | 0.22 | zinc finger and BTB domain containing 38 |
| 222223_s_at | 496.94 | 407.96 | 32.71 | 10.57 | 15.19 | interleukin 1 family, member 5 (delta) |
| 1570238_at | 91.24 | 26.36 | 21.24 | 5.01 | 4.30 | Zinc finger protein 527 |
| 238040_at | 134.06 | 39.10 | 14.12 | 3.95 | 9.50 | Pogo transposable element with ZNF domain |
| 235871_at | 36.41 | 3.75 | 84.12 | 20.46 | 0.43 | lipase, member H |
| 1554878_s_at | 302.06 | 197.00 | 88.47 | 38.40 | 3.41 | ATP-binding cassette, sub-family D (ALD), member 3 |
| 241941_at | 98.12 | 41.36 | 58.18 | 41.77 | 1.69 | Acyl-CoA synthetase bubblegum family member 2 |
| 216231_s_at | 134.94 | 39.54 | 26.41 | 5.27 | 5.11 | beta-2-microglobulin |
| 223133_at | 28.35 | 8.06 | 73.06 | 13.69 | 0.39 | transmembrane protein 14B |
| 228135_at | 15.06 | 4.50 | 190.82 | 60.96 | 0.08 | chromosome 1 open reading frame 52 |
| 1561703_at | 162.00 | 47.90 | 51.88 | 29.21 | 3.12 | CDNA clone IMAGE: 5269594 |
| 1560516_at | 292.06 | 78.66 | 61.94 | 38.04 | 4.72 | Transmembrane protein 26 |
| 223601_at | 23.65 | 6.59 | 96.06 | 18.40 | 0.25 | olfactomedin 2 |
| 218757_s_at | 352.88 | 162.40 | 56.29 | 27.94 | 6.27 | UPF3 regulator of nonsense transcripts homolog B |
| | | | | | | (yeast) |
| 242765_at | 165.88 | 46.45 | 68.65 | 48.12 | 2.42 | myelin-associated oligodendrocyte basic protein |
| 240259_at | 42.82 | 24.15 | 253.35 | 145.04 | 0.17 | |
| 219220_x_at | 31.12 | 11.29 | 121.00 | 40.77 | 0.26 | mitochondrial ribosomal protein S22 |
| 225206_at | 105.65 | 46.45 | 38.71 | 11.93 | 2.73 | Similar to mitochondrial translational release factor 1- |
| | | | | | | like |
| 224050_at | 317.82 | 117.59 | 58.65 | 24.00 | 5.42 | |
| 1562576_at | 173.76 | 98.74 | 46.18 | 20.16 | 3.76 | CDNA clone IMAGE: 5273124 |
| 228697_at | 40.47 | 21.27 | 179.88 | 89.50 | 0.22 | |
| 1560318_at | 109.65 | 32.92 | 31.94 | 9.01 | 3.43 | Rho GTPase activating protein 29 |
| 223008_s_at | 21.29 | 5.79 | 114.12 | 31.08 | 0.19 | chromosome 9 open reading frame 5 |
| 231260_at | 95.41 | 23.66 | 22.18 | 5.91 | 4.30 | hypothetical protein BC036928 |
| 1554963_at | 90.71 | 23.51 | 21.24 | 6.87 | 4.27 | Chromosome 6 open reading frame 192 |
| 240282_at | 23.35 | 12.43 | 173.53 | 54.76 | 0.13 | WD repeat domain 1 |
| 218617_at | 161.94 | 56.90 | 69.65 | 42.24 | 2.33 | tRNA isopentenyltransferase 1 |
| 232476_at | 66.53 | 17.33 | 30.47 | 8.47 | 2.18 | Development and differentiation enhancing factor 2 |
| 226860_at | 35.29 | 21.18 | 107.00 | 32.35 | 0.33 | transmembrane protein 19 |
| 211467_s_at | 51.24 | 33.61 | 164.88 | 52.40 | 0.31 | nuclear factor I/B |
| 212320_at | 38.65 | 19.79 | 139.00 | 53.02 | 0.28 | tubulin, beta polypeptide |
| 218527_at | 132.71 | 46.88 | 105.94 | 72.58 | 1.25 | aprataxin |
| 205836_s_at | 101.47 | 35.87 | 28.82 | 9.64 | 3.52 | YTH domain containing 2 |
| 215468_at | 56.59 | 8.91 | 23.65 | 4.45 | 2.39 | DEAR (Asp-Glu-Ala-His) box polypeptide 9 |
| 210833_at | 123.88 | 43.81 | 42.18 | 21.12 | 2.94 | prostaglandin E receptor 3 (subtype EP3) |
| 240222_at | 90.71 | 35.78 | 20.59 | 6.84 | 4.41 | Transcribed locus, weakly similar to XP 371841.1 |
| | | | | | | PREDICTED: similar to hypothetical protein (L1H 3 |
| | | | | | | region) - human [Homo sapiens] |
| 225426_at | 23.82 | 7.68 | 80.76 | 19.99 | 0.29 | Protein phosphatase 6, catalytic subunit |
| 239962_at | 74.88 | 20.63 | 21.53 | 5.72 | 3.48 | Epidermal growth factor receptor pathway substrate |
| | | | | | | 15-like 1 |
| 222753_s_at | 80.76 | 21.27 | 38.47 | 6.50 | 2.10 | signal peptidase complex subunit 3 homolog (S. cerevisiae) |
| 1557236_at | 49.00 | 11.25 | 21.88 | 3.47 | 2.24 | |
| 220655_at | 127.82 | 53.47 | 38.88 | 23.84 | 3.29 | TNFAIP3 interacting protein 3 |
| 224917_at | 182.71 | 84.41 | 34.82 | 9.97 | 5.25 | microRNA 21 |
| 1562527_at | 101.47 | 25.58 | 27.71 | 8.11 | 3.66 | hypothetical protein LOC283027 |
| 224225_s_at | 101.88 | 39.51 | 45.18 | 10.59 | 2.26 | ets variant gene 7 (TEL2 oncogene) |
| 242844_at | 131.06 | 40.44 | 36.18 | 16.00 | 3.62 | Transcribed locus |
| 206683_at | 180.18 | 84.78 | 53.82 | 21.75 | 3.35 | zinc finger protein 165 |
| 214732_at | 31.94 | 15.62 | 57.47 | 15.94 | 0.56 | Sp1 transcription factor |
| 232776_at | 36.76 | 21.93 | 404.00 | 179.71 | 0.09 | PDZ domain containing RING finger 3 |
| 1552329_at | 161.65 | 58.70 | 25.00 | 8.72 | 6.47 | retinoblastoma binding protein 6 |
| 225658_at | 20.18 | 7.57 | 115.82 | 34.82 | 0.17 | hypothetical protein LOC339745 |
| 225998_at | 31.47 | 17.25 | 105.35 | 40.67 | 0.30 | GRB2-associated binding protein 1 |
| 207920_x_at | 27.59 | 16.59 | 256.76 | 139.55 | 0.11 | zinc finger protein, X-linked |
| 217554_at | 143.76 | 51.35 | 45.65 | 32.61 | 2.96 | Transcribed locus, weakly similar to XP_510104.1 |
| | | | | | | PREDICTED: similar to hypothetical protein |
| | | | | | | FLJ25224 [Pan troglodytes] |
| 215000_s_at | 18.47 | 7.75 | 94.53 | 33.19 | 0.20 | fasciculation and elongation protein zeta 2 (zygin II) |
| 1560533_at | 45.76 | 11.66 | 29.47 | 17.43 | 1.55 | Asparagine-linked glycosylation 14 homolog (yeast) |
| 226007_at | 14.88 | 5.93 | 95.65 | 32.70 | 0.16 | HESB like domain containing 1 |
| 228953_at | 39.53 | 27.84 | 233.71 | 79.31 | 0.17 | similar to junction-mediating and regulatory protein |
| | | | | | | p300 JMY |
| 209243_s_at | 128.35 | 38.39 | 34.71 | 14.66 | 3.70 | paternally expressed 3 |
| 1552486_s_at | 88.53 | 32.66 | 39.00 | 14.37 | 2.27 | lactamase, beta |
| 231169_at | 144.29 | 122.52 | 188.35 | 55.17 | 0.77 | Taxilin alpha |
| 239963_at | 202.24 | 57.59 | 38.71 | 10.81 | 5.22 | Transcribed locus |
| 200636_s_at | 128.18 | 105.73 | 239.71 | 108.80 | 0.53 | protein tyrosine phosphatase, receptor type, F |
| 244180_at | 77.71 | 18.74 | 30.88 | 7.04 | 2.52 | similar to zinc finger protein 569 |
| 214036_at | 40.00 | 16.92 | 79.18 | 20.78 | 0.51 | CDNA: FLJ22256 fis, clone HRC02860 |
| 218677_at | 42.35 | 18.31 | 52.82 | 9.10 | 0.80 | S100 calcium binding protein A14 |
| 1561271_at | 79.88 | 18.81 | 22.12 | 4.06 | 3.61 | CDNA clone MGC: 39818 IMAGE: 5299049 |
| 207889_at | 76.59 | 18.78 | 32.12 | 15.50 | 2.38 | sarcosine dehydrogenase |
| 1559747_at | 57.06 | 20.33 | 13.76 | 4.91 | 4.15 | Hypothetical protein FLJ21439 |
| 218856_at | 83.76 | 53.00 | 188.00 | 65.15 | 0.45 | tumor necrosis factor receptor superfamily member |
| | | | | | | 21 |
| 1553603_s_at | 18.82 | 6.32 | 85.71 | 26.41 | 0.22 | ADP-ribosylation factor-like 6 interacting protein 2 |
| 222525_s_at | 28.76 | 7.75 | 56.47 | 10.96 | 0.51 | coiled-coil domain containing 25 |
| 224209_s_at | 87.59 | 33.00 | 61.65 | 30.62 | 1.42 | guanine deaminase |
| 203512_at | 23.88 | 6.90 | 88.06 | 28.16 | 0.27 | trafficking protein particle complex 3 |
| 217814_at | 52.94 | 36.82 | 182.24 | 73.34 | 0.29 | GK001 protein |
| 231663_s_at | 126.94 | 55.70 | 7.00 | 0.90 | 18.13 | Arginase, liver |
| 242559_at | 15.71 | 4.34 | 113.88 | 37.45 | 0.14 | Transcribed locus |
| 242136_x_at | 78.82 | 18.49 | 34.82 | 13.21 | 2.26 | Hypothetical LOC403340 |
| 203630_s_at | 24.71 | 13.29 | 157.88 | 59.53 | 0.16 | component of oligomeric golgi complex 5 |
| 242121_at | 127.24 | 50.12 | 22.24 | 7.71 | 5.72 | CDNA FLJ33139 fis, clone UTERU1000109 |
| 225364_at | 142.00 | 60.62 | 82.41 | 62.55 | 1.72 | serine/threonine kinase 4 |
| 226476_s_at | 192.24 | 82.31 | 85.94 | 53.81 | 2.24 | Vpr-binding protein |
| 203282_at | 21.41 | 8.51 | 59.71 | 15.10 | 0.36 | glucan (1,4-alpha-), branching enzyme 1 (glycogen |
| | | | | | | branching enzyme, Andersen disease, glycogen |
| | | | | | | storage disease type IV) |
| 219300_s_at | 61.24 | 18.54 | 24.94 | 10.83 | 2.46 | contactin associated protein-like 2 |
| 212166_at | 24.94 | 8.29 | 212.65 | 118.29 | 0.12 | exportin 7 |
| 213914_s_at | 27.00 | 16.76 | 119.35 | 39.87 | 0.23 | Spectrin, beta, non-erythrocytic 1 |
| 1558732_at | 88.35 | 28.80 | 25.12 | 18.62 | 3.52 | |
| 237062_at | 55.76 | 15.09 | 30.76 | 15.02 | 1.81 | |
| 222405_at | 21.00 | 10.95 | 132.24 | 43.95 | 0.16 | butyrate-induced transcript 1 |
| 202037_s_at | 24.94 | 14.61 | 206.59 | 123.13 | 0.12 | secreted frizzled-related protein 1 |
| 219915_s_at | 111.12 | 42.42 | 86.88 | 78.70 | 1.28 | solute carrier family 16 (monocarboxylic acid |
| | | | | | | transporters), member 10 |
| 226756_at | 99.41 | 30.56 | 40.88 | 24.75 | 2.43 | Hypothetical protein FLJ36031 |
| 208781_x_at | 143.76 | 52.41 | 21.41 | 7.33 | 6.71 | sorting nexin 3 |
| 1556069_s_at | 192.12 | 94.35 | 18.65 | 10.71 | 10.30 | Hypoxia inducible factor 3, alpha subunit |
| 220576_at | 132.47 | 45.36 | 20.29 | 8.02 | 6.53 | GPI deacylase |
| 226884_at | 178.59 | 86.68 | 50.53 | 27.78 | 3.53 | leucine rich repeat neuronal 1 |
| 216908_x_at | 53.47 | 14.37 | 19.47 | 5.18 | 2.75 | similar to RNA polymerase I transcription factor |
| | | | | | | RRN3 |
| 210214_s_at | 20.47 | 6.55 | 100.59 | 41.85 | 0.20 | bone morphogenetic protein receptor, type II |
| | | | | | | (serine/threonine kinase) |
| 225681_at | 23.12 | 13.00 | 274.65 | 107.50 | 0.08 | collagen triple helix repeat containing 1 |
| 229905_at | 21.53 | 8.50 | 69.12 | 20.00 | 0.31 | Transcribed locus |
| 231937_at | 67.76 | 19.33 | 20.94 | 4.67 | 3.24 | CDNA FLJ14200 fis, clone NT2RP3002799 |
| 201856_s_at | 21.24 | 7.49 | 106.65 | 34.79 | 0.20 | zinc finger RNA binding protein |
| 236283_x_at | 113.71 | 32.52 | 17.71 | 2.22 | 6.42 | p21 (CDKN1A)-activated kinase 2 |
| 200823_x_at | 22.18 | 9.07 | 47.94 | 11.27 | 0.46 | ribosomal protein L29 |
| 242517_at | 18.24 | 10.99 | 253.94 | 99.70 | 0.07 | G protein-coupled receptor 54 |
| 210296_s_at | 23.76 | 15.23 | 109.71 | 39.15 | 0.22 | peroxisomal membrane protein 3, 35 kDa (Zellweger |
| | | | | | | syndrome) |
| 230332_at | 84.71 | 27.42 | 27.41 | 10.09 | 3.09 | Zinc finger, CCHC domain containing 7 |
| 1557759_at | 102.29 | 33.04 | 31.82 | 9.22 | 3.21 | CDNA FLJ31003 fis, clone HLUNG2000027 |
| 1554747_a_at | 20.88 | 10.40 | 49.71 | 16.12 | 0.42 | septin 2 |
| 212787_at | 21.41 | 7.47 | 119.76 | 44.24 | 0.18 | YLP motif containing 1 |
| 212355_at | 26.94 | 14.10 | 125.76 | 43.29 | 0.21 | KIAA0323 |
| 237563_s_at | 692.06 | 505.59 | 29.47 | 15.91 | 23.48 | LOC440731 |
| 217094_s_at | 133.65 | 46.46 | 43.06 | 20.78 | 3.10 | itchy homolog E3 ubiquitin protein ligase (mouse) |
| 242204_at | 80.71 | 19.49 | 19.29 | 5.51 | 4.18 | WAP four-disulfide core domain 5 |
| 1559449_a_at | 11.06 | 2.93 | 120.53 | 36.08 | 0.09 | Zinc finger protein 254 |
| 201009_s_at | 60.35 | 47.18 | 146.53 | 53.25 | 0.41 | thioredoxin interacting protein |
| 230281_at | 59.29 | 19.13 | 17.94 | 4.67 | 3.30 | chromosome 16 open reading frame 46 |
| 1559545_at | 79.24 | 32.31 | 27.06 | 7.41 | 2.93 | Small nuclear ribonucleoprotein polypeptide N |
| 213624_at | 78.29 | 29.39 | 39.18 | 16.26 | 2.00 | sphingomyelin phosphodiesterase, acid-like 3A |
| 239897_at | 46.53 | 13.51 | 15.88 | 5.56 | 2.93 | BCL2-associated transcription factor 1 |
| 225327_at | 113.71 | 39.46 | 24.88 | 12.80 | 4.57 | hypothetical protein FLJ10980 |
| 212765_at | 56.12 | 41.01 | 98.24 | 39.77 | 0.57 | calmodulin regulated spectrin-associated protein 1- |
| | | | | | | like 1 |
| 220847_x_at | 83.76 | 21.11 | 26.82 | 3.40 | 3.12 | zinc finger protein 221 |
| 226742_at | 12.12 | 3.50 | 95.88 | 33.44 | 0.13 | Transcribed locus, moderately similar to |
| | | | | | | XP_512541.1 PREDICTED: similar to hypothetical |
| | | | | | | protein [Pan troglodytes] |
| 1561112_at | 96.00 | 34.49 | 16.88 | 7.59 | 5.69 | CDNA clone IMAGE: 5299117 |
| 213318_s_at | 52.88 | 13.49 | 28.94 | 3.86 | 1.83 | HLA-B associated transcript 3 |
| 228051_at | 13.29 | 7.30 | 127.35 | 41.54 | 0.10 | hypothetical protein LOC202451 |
| 223529_at | 133.41 | 56.88 | 56.88 | 45.84 | 2.35 | synaptotagmin IV |
| 212340_at | 28.47 | 17.97 | 79.18 | 23.03 | 0.36 | Yip1 domain family, member 6 |
| 1566232_at | 17.35 | 8.56 | 82.41 | 28.05 | 0.21 | MRNA; cDNA DKFZp66710318 (from clone |
| | | | | | | DKFZp66710318) |
| 1563052_at | 53.88 | 11.25 | 18.06 | 4.47 | 2.98 | CDNA clone IMAGE: 5299143 |
| 212109_at | 14.65 | 3.70 | 84.53 | 25.33 | 0.17 | chromosome 16 open reading frame 34 |
| 230175_s_at | 74.18 | 27.74 | 31.65 | 20.11 | 2.34 | |
| 238610_s_at | 46.35 | 12.86 | 16.53 | 5.96 | 2.80 | Heterogeneous nuclear ribonucleoprotein M |
| 209921_at | 101.18 | 49.89 | 20.88 | 12.25 | 4.85 | solute carrier family 7, (cationic amino acid |
| | | | | | | transporter, y+ system) member 11 |
| 242308_at | 76.71 | 27.08 | 15.65 | 9.45 | 4.90 | Mucolipin 3 |
| 219561_at | 52.00 | 33.59 | 72.65 | 21.36 | 0.72 | coatomer protein complex, subunit zeta 2 |
| 225677_at | 8.24 | 2.69 | 75.71 | 30.85 | 0.11 | B-cell receptor-associated protein 29 |
| 227340_s_at | 16.82 | 7.14 | 104.53 | 30.95 | 0.16 | RGM domain family, member B |
| 224693_at | 57.53 | 47.83 | 51.76 | 14.49 | 1.11 | chromosome 20 open reading frame 108 |
| 218764_at | 90.00 | 29.31 | 26.47 | 8.16 | 3.40 | protein kinase C, eta |
| 205027_s_at | 118.94 | 48.83 | 31.12 | 13.57 | 3.82 | mitogen-activated protein kinase kinase kinase 8 |
| 227697_at | 79.35 | 31.04 | 18.41 | 3.95 | 4.31 | suppressor of cytokine signaling 3 |
| 244098_at | 45.41 | 13.09 | 15.06 | 4.70 | 3.02 | ADAM metallopeptidase with thrombospondin type 1 |
| | | | | | | motif, 3 |
| 1554355_a_at | 101.71 | 32.30 | 7.41 | 1.38 | 13.72 | Ysg2 homolog (mouse) |
| 244845_at | 18.47 | 4.56 | 203.88 | 84.53 | 0.09 | CDNA FLJ45435 fis, clone BRHIP3042817 |
| 221905_at | 65.82 | 14.73 | 29.65 | 5.88 | 2.22 | cylindromatosis (turban tumor syndrome) |
| 223467_at | 43.88 | 28.61 | 97.65 | 29.51 | 0.45 | RAS, dexamethasone-induced 1 |
| 241320_at | 109.06 | 43.73 | 12.88 | 6.88 | 8.47 | R3H domain containing 1 |
| 202020_s_at | 12.94 | 7.51 | 277.88 | 140.87 | 0.05 | LanC lantibiotic synthetase component C-like 1 |
| | | | | | | (bacterial) |
| 243888_at | 83.29 | 23.40 | 16.12 | 7.24 | 5.17 | Transcribed locus |
| 201466_s_at | 18.71 | 4.70 | 80.29 | 34.54 | 0.23 | v-jun sarcoma virus 17 oncogene homolog (avian) |
| 1557283_a_at | 47.59 | 9.77 | 12.88 | 3.72 | 3.69 | zinc finger protein 519 |
| 203080_s_at | 12.41 | 3.42 | 46.35 | 11.90 | 0.27 | bromodomain adjacent to zinc finger domain, 2B |
| 230951_at | 52.59 | 19.52 | 14.18 | 5.04 | 3.71 | Erythrocyte membrane protein band 4.1 like 5 |
| 243410_at | 70.24 | 20.28 | 12.41 | 3.85 | 5.66 | Protein tyrosine phosphatase, non-receptor type 2 |
| 210319_x_at | 67.00 | 19.15 | 10.59 | 3.91 | 6.33 | msh homed box homolog 2 (Drosophila) |
| 240246_at | 122.06 | 44.25 | 25.06 | 10.13 | 4.87 | |
| 233430_at | 58.82 | 18.17 | 16.18 | 0.86 | 3.64 | TBC1 domain family, member 22B |
| 201196_s_at | 63.88 | 21.92 | 31.88 | 12.73 | 2.00 | adenosylmethionine decarboxylase 1 |
| 236236_at | 64.35 | 19.22 | 6.47 | 0.80 | 9.95 | CDNA FLJ30437 fis, clone BRACE2009045 |
| 222316_at | 87.76 | 36.75 | 16.82 | 7.42 | 5.22 | Vesicle docking protein p115 |
| 223506_at | 50.71 | 12.07 | 16.29 | 4.17 | 3.11 | zinc finger CCCH-type containing 8 |
| 244755_at | 72.29 | 27.61 | 24.06 | 10.14 | 3.00 | |
| 235955_at | 17.94 | 7.59 | 128.88 | 49.51 | 0.14 | MARVEL domain containing 2 |
| 1557570_a_at | 130.59 | 57.62 | 16.88 | 10.46 | 7.74 | hypothetical protein LOC285084 |
| 201370_s_at | 104.59 | 42.35 | 33.94 | 17.11 | 3.08 | cullin 3 |
| 1568794_at | 167.00 | 64.27 | 10.71 | 3.03 | 15.60 | CDNA clone IMAGE: 5277859 |
| 231747_at | 70.65 | 19.17 | 17.00 | 6.61 | 4.16 | cysteinyl leukotriene receptor 1 |
| 215975_x_at | 110.53 | 45.29 | 19.82 | 9.82 | 5.58 | glycerol kinase |
| 236409_at | 20.18 | 8.84 | 183.53 | 92.61 | 0.11 | Lysophospholipase-like 1 |

TABLE 4
Biological functions of over-represented gene ontology attributes
if differentially expressed genes between IN and NS at day 1

| | # of | # of | | | | |
|---|---|---|---|---|---|---|
| | genes/ | genes/ | | Gene | Gene | majority of |
| No. | 806 genes | genome | p value | Ontology-ID | Ontology-Attribute | Genes/IN |
| 1 | 9 | 20 | 2.30E−09 | GO:0001533 | cornified envelope | over- |
| | | | | | | expressed |
| 2 | 5 | 13 | 2.50E−05 | GO:0000276 | mitochondrial proton-transporting | down- |
| | | | | | ATP synthase complex | regulation |
| 3 | 9 | 29 | 1.08E−07 | GO:0031424 | keratinization | over- |
| | | | | | | expressed |
| 4 | 14 | 51 | 1.92E−10 | GO:0030216 | keratinocyte differentiation | over- |
| | | | | | | expressed |
| 5 | 7 | 30 | 2.34E−05 | GO:0055010 | ventricular cardiac muscle | down- |
| | | | | | morphogenesis | regulation |
| 6 | 28 | 162 | 6.16E−14 | GO:0008544 | epidermis development | down- |
| | | | | | | regulation |
| 7 | 20 | 141 | 8.46E−09 | GO:0005200 | structural constituent of cytoskeleton | down- |
| | | | | | | regulation |
| 8 | 14 | 115 | 9.00E−06 | GO:0004867 | serine-type endopeptidase inhibitor | down- |
| | | | | | activity | regulation |
| 9 | 16 | 144 | 6.96E−06 | GO:0005882 | intermediate filament | down- |
| | | | | | | regulation |
| 10 | 20 | 194 | 1.69E−06 | GO:0004866 | endopeptidase inhibitor activity | down- |
| | | | | | | regulation |
| 11 | 20 | 197 | 2.14E−06 | GO:0030414 | peptidase inhibitor activity | down- |
| | | | | | | regulation |
| 12 | 37 | 367 | 1.40E−10 | GO:0009888 | tissue development | down- |
| | | | | | | regulation |
| 13 | 32 | 381 | 1.86E−07 | GO:0004857 | enzyme inhibitor activity | down- |
| | | | | | | regulation |
| 14 | 55 | 949 | 2.66E−06 | GO:0005198 | structural molecule activity | down- |
| | | | | | | regulation |

TABLE 5
Number of genes whose expression profile correlated
with Duac treatment in over-represented GO attributes

| | # of genes | | | | majority of | majority of |
|---|---|---|---|---|---|---|
| | correlated | # of | # of | | genes/IN | genes/IN |
| | with Duac | genes/ | genes/ | Gene | compared | during Duac |
| No. | treatment | 806 genes | genome | Ontology-Attribute | to NS | treatment |
| 1 | 3 | 9 | 20 | cornified envelope mitochondrial | over- | decreased |
| | | | | proton-transporting ATP | expressed | |
| 2 | 5 | 5 | 13 | synthase complex | down- | increased |
| | | | | | regulation | |
| 3 | 3 | 9 | 29 | keratinization | over- | decreased |
| | | | | | expressed | |
| 4 | 5 | 14 | 51 | keratinocyte differentiation | over- | decreased |
| | | | | | expressed | |
| 5 | 3 | 7 | 30 | ventricular cardiac muscle | down- | increased |
| | | | | morphogenesis | regulation | |
| 6 | 15 | 28 | 162 | epidermis development | down- | increased |
| | | | | | regulation | |
| 7 | 7 | 20 | 141 | structural constituent of | down- | increased |
| | | | | cytoskeleton | regulation | |
| 8 | 5 | 14 | 115 | serine-type endopeptidase inhibitor | down- | increased |
| | | | | activity | regulation | |
| 9 | 9 | 16 | 144 | intermediate filament | down- | increased |
| | | | | | regulation | |
| 10 | 10 | 20 | 194 | endopeptidase inhibitor activity | down- | increased |
| | | | | | regulation | |
| 11 | 10 | 20 | 197 | peptidase inhibitor activity | down- | increased |
| | | | | | regulation | |
| 12 | 20 | 37 | 367 | tissue development | down- | increased |
| | | | | | regulation | |
| 13 | 10 | 32 | 381 | enzyme inhibitor activity | down- | increased |
| | | | | | regulation | |
| 14 | 32 | 55 | 949 | structural molecule activity | down- | increased |
| | | | | | regulation | |

TABLE 6
Average expression data of 126 genes between inflammatory acne lesions and
normal skin controls at day 1 and treatment of Duac from 17 patients

| | Duac treatment | | | | | |
|---|---|---|---|---|---|---|
| | None | None | Duac | Duac | Duac | |
| probeset | NS-day 1 | IN-day 1 | IN-week 2 | IN-week 5 | IN-week 8 | Description |
| 207356_at | 164 | 2912 | 1816 | 956 | 2273 | defensin, beta 4 |
| 210319_x_at | 10 | 66 | 34 | 15 | 14 | msh homeo box homolog 2 (Drosophila) |
| 231747_at | 14 | 136 | 118 | 38 | 36 | cysteinyl leukotriene receptor 1 |
| 1561112_at | 16 | 91 | 327 | 17 | 20 | CDNA clone IMAGE: 5299117 |
| 218340_s_at | 34 | 178 | 150 | 62 | 94 | hypothetical protein FLJ10808 |
| 230332_at | 25 | 82 | 29 | 16 | 26 | Zinc finger, CCHC domain containing 7 |
| 1563660_at | 41 | 106 | 67 | 51 | 38 | MRNA; cDNA DKFZp686A0837 (from clone DKFZp686A0837) |
| 203744_at | 112 | 253 | 97 | 125 | 109 | high-mobility group box 3 |
| 218050_at | 468 | 797 | 507 | 427 | 452 | ubiquitin-fold modifier 1 |
| 202859_x_at | 112 | 956 | 481 | 729 | 278 | interleukin 8 |
| 1554355_a_at | 17 | 79 | 39 | 22 | 54 | Ysg2 homolog (mouse) |
| 1554963_at | 21 | 134 | 57 | 62 | 78 | Chromosome 6 open reading frame 192 |
| 1559545_at | 25 | 76 | 18 | 27 | 34 | Small nuclear ribonucleoprotein polypeptide N |
| 236236_at | 6 | 61 | 37 | 81 | 190 | CDNA FLJ30437 fis, clone BRACE2009045 |
| 1560533_at | 30 | 48 | 16 | 20 | 41 | Asparagine-linked glycosylation 14 homolog (yeast) |
| 217739_s_at | 139 | 846 | 545 | 1062 | 331 | pre-B-cell colony enhancing factor 1 |
| 222316_at | 17 | 83 | 50 | 36 | 49 | Vesicle docking protein p115 |
| 218422_s_at | 80 | 125 | 61 | 62 | 88 | chromosome 13 open reading frame 10 |
| 1559492_at | 188 | 822 | 102 | 294 | 180 | CDNA clone IMAGE: 5268696 |
| 1559747_at | 49 | 96 | 35 | 62 | 114 | Hypothetical protein FLJ21439 |
| 230281_at | 17 | 59 | 13 | 19 | 38 | chromosome 16 open reading frame 46 |
| 215936_s_at | 29 | 137 | 61 | 97 | 65 | KIAA1033 |
| 220576_at | 17 | 127 | 34 | 30 | 26 | GPI deacylase |
| 232476_at | 31 | 76 | 54 | 37 | 37 | Development and differentiation enhancing factor 2 |
| 236283_x_at | 21 | 152 | 37 | 89 | 70 | p21 (CDKN1A)-activated kinase 2 |
| 203691_at | 189 | 2073 | 2456 | 1533 | 1811 | peptidase inhibitor 3, skin-derived (SKALP); peptidase inhibitor 3, skin- |
| | | | | | | derived (SKALP) |
| 202912_at | 49 | 207 | 267 | 170 | 180 | adrenomedullin |
| 41469_at | 18 | 359 | 463 | 273 | 343 | peptidase inhibitor 3, skin-derived (SKALP) |
| 202464_s_at | 1233 | 3816 | 4220 | 4173 | 3860 | 6-phosphofructo-2-kinase/fructose-2,6-biphosphatase 3 |
| 203725_at | 202 | 1860 | 2786 | 1590 | 1967 | growth arrest and DNA-damage-inducible, alpha |
| 209595_at | 160 | 268 | 352 | 177 | 199 | general transcription factor IIF, polypeptide 2, 30 kDa |
| 227697_at | 18 | 79 | 55 | 68 | 31 | suppressor of cytokine signaling 3 |
| 205027_s_at | 29 | 112 | 139 | 131 | 126 | mitogen-activated protein kinase kinase kinase 8 |
| 208539_x_at | 881 | 4562 | 6229 | 3815 | 5131 | small proline-rich protein 28 |
| 236119_s_at | 536 | 3946 | 5119 | 3302 | 3961 | small proline-rich protein 2G |
| 244087_at | 46 | 171 | 270 | 87 | 126 | Adenosine monophosphate deaminase (isoform E) |
| 220847_x_at | 28 | 74 | 83 | 59 | 58 | zinc finger protein 221 |
| 236243_at | 229 | 351 | 505 | 279 | 328 | Zinc finger, CCHC domain containing 6 |
| 241904_at | 52 | 89 | 105 | 74 | 86 | Lipase, hormone-sensitive |
| 208901_s_at | 201 | 483 | 371 | 257 | 436 | topoisomerase (DNA) I |
| 205178_s_at | 520 | 1029 | 1401 | 1561 | 1586 | retinoblastoma binding protein 6 |
| 209719_x_at | 21 | 1417 | 1597 | 582 | 1175 | serpin peptidase inhibitor, clade B (ovalbumin), member 3 |
| 208810_at | 142 | 881 | 1218 | 468 | 809 | DnaJ (Hsp40) homolog, subfamily B, member 6 |
| 216231_s_at | 27 | 127 | 75 | 50 | 71 | beta-2-microglobulin |
| 200776_s_at | 88 | 328 | 394 | 84 | 229 | basic leucine zipper and W2 domains 1; similar to basic leucine zipper |
| | | | | | | and W2 domains 1 |
| 201437_s_at | 489 | 644 | 713 | 571 | 740 | eukaryotic translation initiation factor 4E |
| 212530_at | 184 | 343 | 419 | 198 | 335 | NIMA (never in mitosis gene a)-related kinase 7 |
| 202147_s_at | 140 | 666 | 765 | 506 | 609 | interferon-related developmental regulator 1 |
| 241320_at | 13 | 103 | 35 | 24 | 59 | R3H domain containing 1 |
| 218527_at | 99 | 130 | 173 | 25 | 32 | aprataxin |
| 1554868_s_at | 92 | 181 | 163 | 94 | 73 | PEST-containing nuclear protein |
| 219915_s_at | 85 | 144 | 175 | 33 | 51 | solute carrier family 16 (monocarboxylic acid transporters), member 10 |
| 201001_s_at | 1240 | 1905 | 1126 | 911 | 946 | ubiquitin-conjugating enzyme E2 variant 1; ubiquitin-conjugating |
| | | | | | | enzyme E2 variant 1 |
| 207731_at | 243 | 458 | 696 | 169 | 140 | |
| 1554996_at | 35 | 182 | 215 | 26 | 59 | zinc finger protein 479 |
| 1566144_at | 377 | 1027 | 439 | 428 | 401 | SH3-domain GRB2-like 3 |
| 1561703_at | 54 | 147 | 106 | 109 | 60 | CDNA clone IMAGE: 5269594 |
| 231809_x_at | 156 | 916 | 270 | 181 | 141 | programmed cell death 7 |
| 209243_s_at | 32 | 121 | 58 | 32 | 24 | paternally expressed 3 |
| 237563_s_at | 29 | 673 | 102 | 71 | 141 | LOC440731 |
| 1570361_a_at | 61 | 197 | 112 | 87 | 50 | Homo sapiens, clone IMAGE: 3935253, mRNA |
| 216591_s_at | 89 | 225 | 313 | 91 | 132 | succinate dehydrogenase complex, subunit C, integral membrane protein, |
| | | | | | | 15 kDa |
| 232500_at | 39 | 112 | 120 | 77 | 33 | chromosome 20 open reading frame 74 |
| 230951_at | 14 | 52 | 11 | 5 | 15 | Erythrocyte membrane protein band 4.1 like 5 |
| 240901_at | 62 | 242 | 41 | 44 | 34 | Transcribed locus |
| 229630_s_at | 714 | 1543 | 866 | 920 | 763 | Wilms tumor 1 associated protein |
| 223529_at | 56 | 127 | 50 | 26 | 28 | synaptotagmin IV |
| 1561155_at | 66 | 255 | 84 | 48 | 46 | Cation channel, sperm associated 2 |
| 232281_at | 50 | 205 | 232 | 91 | 78 | CDNA FLJ12237 fis, clone MAMMA1001249 |
| 231969_at | 28 | 118 | 59 | 44 | 43 | storkhead box 2 |
| 237062_at | 30 | 55 | 39 | 17 | 47 | |
| 231937_at | 21 | 64 | 39 | 36 | 44 | CDNA FLJ14200 fis, clone NT2RP3002799 |
| 230175_s_at | 31 | 73 | 39 | 36 | 44 | |
| 222378_at | 59 | 233 | 90 | 108 | 125 | Hypothetical protein FLJ43663 |
| 238040_at | 26 | 157 | 56 | 135 | 137 | Pogo transposable element with ZNF domain |
| 1570238_at | 21 | 98 | 29 | 37 | 29 | Zinc finger protein 527 |
| 239897_at | 15 | 44 | 8 | 52 | 59 | BCL2-associated transcription factor 1 |
| 242121_at | 21 | 118 | 136 | 60 | 115 | CDNA FLJ33139 fis, clone UTERU1000109 |
| 244016_at | 41 | 220 | 73 | 89 | 49 | Transcribed locus |
| 1567213_at | 45 | 196 | 98 | 79 | 66 | pinin, desmosome associated protein |
| 1552946_at | 21 | 121 | 37 | 52 | 95 | zinc finger protein 114 |
| 1553412_at | 83 | 353 | 232 | 198 | 205 | ATP/GTP binding protein-like 4 |
| 241786_at | 52 | 76 | 42 | 73 | 32 | Protein phosphatase 3 (formerly 2B), regulatory subunit B, 19 kDa, alpha |
| | | | | | | isoform (calcineurin B, type I) |
| 215468_at | 21 | 62 | 25 | 31 | 28 | DEAH (Asp-Glu-Ala-His) box polypeptide 9 |
| 231260_at | 25 | 79 | 59 | 62 | 65 | hypothetical protein BC036928 |
| 209921_at | 20 | 93 | 56 | 48 | 53 | solute carrier family 7, (cationic amino acid transporter, y+ system) |
| | | | | | | member 11 |
| 1570593_at | 75 | 174 | 63 | 95 | 108 | ATPase, aminophospholipid transporter (APLT), Class I, type 8A, |
| | | | | | | member 1 |
| 244755_at | 22 | 72 | 10 | 48 | 69 | |
| 1557433_at | 34 | 92 | 69 | 34 | 37 | |
| 240502_at | 61 | 326 | 111 | 137 | 182 | FERM domain containing 5 |
| 239860_at | 171 | 645 | 142 | 256 | 470 | Hypothetical protein PRO2949 |
| 233993_at | 770 | 1822 | 950 | 1661 | 1009 | SIB 297 intestinal mucin (MUC3) |
| 229027_at | 218 | 484 | 264 | 202 | 273 | Protein phosphatase 1A (formerly 2C), magnesium-dependent, alpha |
| | | | | | | isoform |
| 1566146_x_at | 386 | 1078 | 424 | 508 | 437 | SH3-domain GRB2-like 3 |
| 1569906_s_at | 157 | 368 | 129 | 372 | 276 | PHD finger protein 20 |
| 224917_at | 30 | 173 | 29 | 120 | 43 | microRNA 21 |
| 226476_s_at | 84 | 205 | 40 | 31 | 133 | Vpr-binding protein |
| 1561271_at | 20 | 85 | 25 | 95 | 46 | CDNA clone MGC: 39818 IMAGE: 5299049 |
| 1561737_at | 55 | 125 | 85 | 87 | 97 | |
| 201699_at | 317 | 1153 | 1220 | 747 | 934 | proteasome (prosome, macropain) 265 subunit, ATPase, 6 |
| 202769_at | 60 | 192 | 243 | 83 | 196 | Cyclin G2 |
| 227638_at | 103 | 386 | 743 | 450 | 701 | KIAA1632 |
| 206643_at | 53 | 891 | 2215 | 1561 | 1731 | histidine ammonia-lyase |
| 222753_s_at | 39 | 83 | 71 | 39 | 102 | signal peptidase complex subunit 3 homolog (S. cerevisiae) |
| 1569476_at | 51 | 136 | 135 | 104 | 172 | DKFZP434L187 protein |
| 217557_s_at | 440 | 1142 | 2608 | 1469 | 1065 | |
| 224328_s_at | 890 | 5244 | 6538 | 4126 | 6313 | late cornified envelope 3D; late cornified envelope 3D |
| 201266_at | 56 | 298 | 594 | 283 | 301 | thioredoxin reductase 1 |
| 1569200_at | 46 | 197 | 119 | 89 | 114 | SEC15-like 2 (S. cerevisiae) |
| 1554878_a_at | 66 | 308 | 445 | 317 | 280 | ATP-binding cassette, sub-family D (ALD), member 3 |
| 210378_s_at | 93 | 131 | 122 | 109 | 122 | Sjogren's syndrome nuclear autoantigen 1 |
| 209365_s_at | 46 | 215 | 896 | 281 | 345 | extracellular matrix protein 1 |
| 201944_at | 611 | 1237 | 3322 | 2393 | 2999 | hexosaminidase B (beta polypeptide) |
| 232220_at | 201 | 1216 | 606 | 640 | 1260 | |
| 206177_s_at | 81 | 446 | 551 | 590 | 956 | arginase, liver |
| 1565666_s_at | 1397 | 3214 | 2902 | 3789 | 2848 | mucin 6, gastric |
| 206683_at | 44 | 177 | 411 | 662 | 384 | zinc finger protein 165 |
| 1558732_at | 26 | 87 | 46 | 54 | 238 | |
| 201370_s_at | 34 | 104 | 76 | 47 | 74 | cullin 3 |
| 218309_at | 101 | 203 | 357 | 163 | 298 | calcium/calmodulin-dependent protein kinase II inhibitor 1 |
| 202917_s_at | 968 | 6393 | 4708 | 2940 | 4185 | 5100 calcium binding protein A8 (calgranulin A) |
| 218310_at | 54 | 200 | 242 | 137 | 211 | RAB guanine nucleotide exchange factor (GEF) 1 |
| 232082_x_at | 171 | 1165 | 1852 | 1026 | 1377 | small proline-rich protein 3 |
| 208781_x_at | 21 | 142 | 55 | 22 | 71 | sorting nexin 3 |
| 201196_s_at | 28 | 63 | 56 | 40 | 114 | adenosylmethionine decarboxylase 1 |
| 204351_at | 280 | 838 | 1300 | 759 | 1162 | S100 calcium binding protein P |

TABLE 7
Average expression data of 119 genes between inflammatory acne lesions and
normal skin controls at day 1 and treatment of Duac from 17 patients

| | Duac treatment | | | | | |
|---|---|---|---|---|---|---|
| | None | None | Duac | Duac | Duac | |
| probeset | NS-day 1 | IN-day 1 | IN-week 2 | IN-week 5 | IN-week 8 | Description |
| 1552620_at | 7872 | 3325 | 3777 | 2753 | 4905 | small proline rich protein 4 |
| 1553602_at | 9321 | 3598 | 3806 | 2338 | 5082 | small breast epithelial mucin |
| 1554678_s_at | 1479 | 832 | 785 | 652 | 998 | heterogeneous nuclear ribonucleoprotein D-like |
| 1555961_a_at | 1659 | 1111 | 1175 | 1050 | 1052 | histidine triad nucleotide binding protein 1 |
| 1558378_a_at | 5332 | 3288 | 2584 | 2720 | 4071 | chromosome 14 open reading frame 78 |
| 1558924_s_at | 2507 | 1494 | 2786 | 1579 | 2512 | restin (Reed-Steinberg cell-expressed intermediate filament-associated |
| | | | | | | protein) |
| 200673_at | 2010 | 1247 | 1241 | 941 | 1316 | lysosomal-associated protein transmembrane 4 alpha |
| 200696_s_at | 913 | 422 | 409 | 294 | 584 | gelsolin (amyloidosis, Finnish type) |
| 200847_s_at | 1260 | 821 | 919 | 744 | 1197 | transmembrane protein 66 |
| 201008_s_at | 1229 | 377 | 419 | 222 | 411 | thioredoxin interacting protein |
| 201010_s_at | 1578 | 510 | 675 | 436 | 552 | thioredoxin interacting protein |
| 201130_s_at | 276 | 73 | 64 | 118 | 87 | cadherin 1, type 1, E-cadherin (epithelial) |
| 201147_s_at | 911 | 491 | 637 | 465 | 772 | TIMP metallopeptidase inhibitor 3 (Sorsby fundus dystrophy, |
| | | | | | | pseudoinflammatory) |
| 201150_s_at | 214 | 87 | 174 | 63 | 201 | TIMP metallopeptidase inhibitor 3 (Sorsby fundus dystrophy, |
| | | | | | | pseudoinflammatory) |
| 201223_s_at | 1328 | 431 | 722 | 647 | 555 | RAD23 homolog B (S. cerevisiae) |
| 201260_s_at | 1396 | 962 | 861 | 690 | 1027 | synaptophysin-like 1 |
| 201286_at | 701 | 168 | 252 | 102 | 269 | syndecan 1 |
| 201287_s_at | 286 | 125 | 135 | 61 | 118 | syndecan 1 |
| 201667_at | 5817 | 3953 | 3594 | 3518 | 4029 | gap junction protein, alpha 1, 43 kDa (connexin 43) |
| 201820 at | 1790 | 608 | 290 | 209 | 1058 | keratin 5 (epidermolysis bullosa simplex, Dowling- |
| | | | | | | Meara/Kobner/Weber-Cockayne types) |
| 202053_s_at | 3669 | 884 | 1700 | 1080 | 1723 | aldehyde dehydrogenase 3 family, member A2 |
| 202054_s_at | 1000 | 462 | 406 | 354 | 427 | aldehyde dehydrogenase 3 family, member A2 |
| 202376_at | 413 | 378 | 261 | 171 | 340 | serpin peptidase inhibitor, clade A (alpha-1 antiproteinase, antitrypsin), |
| | | | | | | member 3 |
| 202575_at | 1947 | 939 | 1094 | 1087 | 1002 | cellular retinoic acid binding protein 2 |
| 202593_s_at | 1020 | 631 | 1867 | 837 | 1346 | membrane interacting protein of RGS16 |
| 202753_at | 601 | 521 | 624 | 273 | 381 | proteasome (prosome, macropain) 26S subunit, non-ATPase, 6 |
| 203076_s_at | 1318 | 335 | 529 | 385 | 541 | SMAD, mothers against DPP homolog 2 (Drosophila) |
| 203123_s_at | 1913 | 1289 | 1911 | 894 | 1338 | solute carrier family 11 (proton-coupled divalent metal ion transporters), |
| | | | | | | member 2 |
| 203124_s_at | 877 | 322 | 582 | 239 | 475 | solute carrier family 11 (proton-coupled divalent metal ion transporters), |
| | | | | | | member 2 |
| 203126_at | 465 | 222 | 152 | 189 | 359 | inositol(myo)-1(or 4)-monophosphatase 2 |
| 203263_s_at | 226 | 67 | 118 | 100 | 259 | Cdc42 guanine nucleotide exchange factor (GEF) 9 |
| 203430_at | 1896 | 1135 | 1948 | 1718 | 1996 | heme binding protein 2 |
| 204589_at | 512 | 133 | 314 | 96 | 372 | NUAK family, SNF1-like kinase, 1 |
| 204734_at | 2296 | 690 | 1203 | 693 | 1398 | keratin 15 |
| 204818_at | 562 | 226 | 307 | 115 | 313 | hydroxysteroid (17-beta) dehydrogenase 2 |
| 205157_s_at | 8855 | 4483 | 4460 | 3157 | 5075 | keratin 17 |
| 205185_at | 10270 | 5656 | 7687 | 5322 | 7485 | serine peptidase inhibitor, Kazal type 5 |
| 206011_at | 296 | 79 | 222 | 43 | 189 | caspase 1, apoptosis-related cysteine peptidase (interleukin 1, beta, |
| | | | | | | convertase) |
| 206116_s_at | 3327 | 1078 | 1379 | 1193 | 2482 | tropomyosin 1 (alpha) |
| 206605_at | 2573 | 1036 | 1550 | 1260 | 1676 | 26 serine protease |
| 206642_at | 1974 | 1170 | 1167 | 962 | 1677 | desmoglein 1 |
| 207065_at | 1220 | 728 | 1295 | 777 | 1341 | cytokeratin type II |
| 207920_x_at | 248 | 28 | 44 | 24 | 114 | zinc finger protein, X-linked |
| 208892_s_at | 735 | 360 | 343 | 317 | 449 | dual specificity phosphatase 6 |
| 209126_x_at | 4056 | 2635 | 2254 | 1631 | 2145 | keratin 6B |
| 209234_at | 816 | 375 | 1504 | 1188 | 1200 | kinesin family member 1B |
| 209240_at | 129 | 52 | 65 | 52 | 154 | O-linked N-acetylglucosamine (GlcNAc) transferase (UDP-N- |
| | | | | | | acetylglucosamine: polypeptide-N-acetylglucosaminyl transferase) |
| 209283_at | 2796 | 1778 | 2003 | 1334 | 2148 | crystallin, alpha B |
| 209351_at | 3558 | 1397 | 1145 | 794 | 1765 | keratin 14 (epidermolysis bullosa simplex, Dowling-Meara, Koebner) |
| 209590_at | 155 | 95 | 70 | 28 | 171 | Bone morphogenetic protein 7 (osteogenic protein 1) |
| 209688_s_at | 168 | 55 | 22 | 42 | 110 | hypothetical protein FLJ10996 |
| 209800_at | 2989 | 1483 | 1583 | 1045 | 1600 | keratin 16 (focal non-epidermolytic palmoplantar keratoderma) |
| 210074_at | 11288 | 6755 | 7607 | 5498 | 8693 | cathepsin L2 |
| 210186_s_at | 412 | 102 | 227 | 118 | 341 | FK506 binding protein 1A, 12 kDa |
| 211002_s_at | 1439 | 841 | 579 | 488 | 613 | tripartite motif-containing 29 |
| 211296_x_at | 4096 | 2734 | 3004 | 2177 | 3047 | ubiquitin C |
| 211345_x_at | 3561 | 2492 | 3500 | 3111 | 3767 | eukaryotic translation elongation factor 1 gamma |
| 212062_at | 305 | 97 | 124 | 172 | 190 | ATPase, Class II, type 9A |
| 212077_at | 378 | 28 | 53 | 10 | 72 | caldesmon 1 |
| 212115_at | 601 | 273 | 676 | 636 | 651 | chromosome 16 open reading frame 34 |
| 212236_x_at | 9794 | 4552 | 4901 | 3666 | 6247 | keratin 17 |
| 212566_at | 2252 | 978 | 1295 | 1107 | 1692 | microtubule-associated protein 4 |
| 212593_s_at | 5943 | 3137 | 2851 | 3152 | 4226 | programmed cell death 4 (neoplastic transformation inhibitor) |
| 212826_s_at | 899 | 470 | 555 | 391 | 724 | solute carrier family 25 (mitochondria) carrier; adenine nucleotide |
| | | | | | | translocator), member 6 |
| 212904_at | 130 | 14 | 110 | 31 | 71 | leucine rich repeat containing 47 |
| 212915_at | 223 | 83 | 110 | 126 | 89 | PDZ domain containing RING finger 3 |
| 213260_at | 1231 | 771 | 1037 | 546 | 1050 | Forkhead box C1 |
| 214119_s_at | 797 | 286 | 628 | 313 | 653 | FK506 binding protein 1A, 12 kDa |
| 215000_s_at | 122 | 19 | 15 | 24 | 44 | fasciculation and elongation protein zeta 2 (zygin II) |
| 215424_s_at | 385 | 102 | 99 | 118 | 286 | SNW domain containing 1 |
| 217234_s_at | 835 | 638 | 525 | 264 | 1061 | villin 2 (ezrin) |
| 217717_s_at | 595 | 167 | 362 | 169 | 247 | tyrosine 3-monooxygenase/tryptophan 5-monooxygenase activation |
| | | | | | | protein, beta polypeptide |
| 217744_s at | 1687 | 987 | 1300 | 756 | 1148 | PERP, TP53 apoptosis effector |
| 217769_s_at | 2967 | 1758 | 1861 | 1543 | 2092 | chromosome 13 open reading frame 12 |
| 217807_s_at | 1931 | 372 | 821 | 580 | 1506 | glioma tumor suppressor candidate region gene 2 |
| 217917_s_at | 531 | 357 | 569 | 473 | 658 | dynein, cytoplasmic, light polypeptide 2A |
| 217918_at | 904 | 548 | 897 | 798 | 777 | dynein, cytoplasmic, light polypeptide 2A |
| 218816_at | 307 | 47 | 92 | 34 | 108 | leucine rich repeat containing 1 |
| 219410_at | 6322 | 3840 | 4169 | 3435 | 4454 | transmembrane protein 45A |
| 222404_x_at | 521 | 77 | 421 | 66 | 394 | butyrate-induced transcript 1 |
| 223044_at | 730 | 182 | 470 | 474 | 494 | solute carrier family 40 (iron-regulated transporter), member 1 |
| 223449_at | 198 | 39 | 68 | 36 | 119 | sema domain, transmembrane domain (TM), and cytoplasmic domain, |
| | | | | | | (semaphorin) 6A |
| 224367_at | 884 | 419 | 854 | 464 | 897 | brain expressed X-linked 2; brain expressed X-linked 2 |
| 224570_s_at | 1158 | 485 | 505 | 409 | 574 | interferon regulatory factor 2 binding protein 2 |
| 224602 at | 2841 | 1398 | 2628 | 1386 | 2252 | HCV F-transactivated protein 1 |
| 225117_at | 220 | 64 | 176 | 90 | 123 | LOC284058 protein |
| 225345_s_at | 1167 | 274 | 424 | 421 | 608 | F-box protein 32 |
| 225615_at | 996 | 454 | 855 | 655 | 926 | hypothetical protein LOC126917 |
| 225629_s_at | 2019 | 373 | 464 | 536 | 1504 | zinc finger and BTB domain containing 4 |
| 226213_at | 1872 | 977 | 917 | 417 | 919 | v-erb-b2 erythroblastic leukemia viral oncogene homolog 3 (avian) |
| 226614_s_at | 261 | 125 | 92 | 65 | 112 | chromosome 8 open reading frame 13 |
| 227036_at | 122 | 46 | 41 | 33 | 106 | Transcribed locus |
| 228496_s_at | 292 | 90 | 141 | 47 | 142 | Cysteine rich transmembrane BMP regulator 1 (chordin-like) |
| 228575_at | 1602 | 532 | 1909 | 1037 | 994 | fibronectin type III domain containing 6 |
| 228926_s_at | 112 | 39 | 33 | 18 | 90 | SWI/SNF related, matrix associated, actin dependent regulator of |
| | | | | | | chromatin, subfamily a, member 2 |
| 228993_s_at | 6504 | 3682 | 3205 | 3312 | 4783 | hypothetical protein LOC92482 |
| 230291_s_at | 838 | 304 | 500 | 239 | 539 | Nuclear factor I/B |
| 230788_at | 1007 | 120 | 302 | 83 | 342 | glucosaminyl (N-acetyl) transferase 2, I-branching enzyme |
| 231211_s_at | 447 | 220 | 674 | 320 | 579 | hypothetical LOC541469 protein |
| 232602_at | 1649 | 629 | 303 | 367 | 687 | WAP four-disulfide core domain 3 |
| 233641_s_at | 903 | 182 | 416 | 107 | 459 | Chromosome 8 open reading frame 13 |
| 234000_s_at | 267 | 44 | 187 | 33 | 198 | butyrate-induced transcript 1 |
| 236534_at | 622 | 440 | 806 | 296 | 464 | BCL2/adenovirus E1B 19 kD interacting protein like |
| 237030_at | 703 | 256 | 379 | 419 | 543 | acid phosphatase, prostate |
| 237120_at | 4894 | 3519 | 4185 | 3923 | 4564 | keratin 1B |
| 237690 at | 798 | 375 | 456 | 432 | 536 | G protein-coupled receptor 115 |
| 237732_at | 641 | 563 | 489 | 678 | 824 | Transcribed locus, moderately similar to NP_780633.1 hypothetical |
| | | | | | | protein LOC109314 [Mus musculus] |
| 238041_at | 154 | 29 | 72 | 14 | 61 | Transcription factor 12 (HTF4, helix-loop-helix transcription factors 4) |
| 238427_at | 569 | 151 | 451 | 286 | 259 | GrpE-like 2, mitochondrial (E. coli) |
| 239321_at | 224 | 24 | 23 | 15 | 69 | Hypothetical gene supported by BC013859 |
| 239377 at | 2961 | 1374 | 2079 | 1452 | 1811 | hypothetical protein MGC11102 |
| 239719_at | 209 | 81 | 98 | 23 | 139 | CD109 antigen (Gov platelet alloantigens) |
| 39248_at | 13686 | 9115 | 8561 | 9134 | 9687 | aquaporin 3 |
| 200602_at | 851 | 223 | 394 | 118 | 489 | amyloid beta (A4) precursor protein (peptidase nexin-II, Alzheimer |
| | | | | | | disease) |
| 201149_s_at | 353 | 84 | 153 | 25 | 302 | TIMP metallopeptidase inhibitor 3 (Sorsby fundus dystrophy, |
| | | | | | | pseudoinflammatory) |
| 202551_s_at | 310 | 94 | 229 | 73 | 195 | cysteine rich transmembrane BMP regulator 1 (chordin-like) |
| 206595_at | 11860 | 4350 | 6518 | 4468 | 7425 | cystatin E/M |
| 210715_s_at | 1053 | 732 | 782 | 565 | 792 | serine peptidase inhibitor, Kunitz type, 2 |
| 231733_at | 3406 | 1312 | 1857 | 1230 | 2276 | ICEBERG caspase-1 inhibitor |

TABLE 8
Average expression data of inflammatory acne lesions and normal skin controls at day 1 and treatment of Duac from 17 patients

| | Duac treatment | | | | | | |
|---|---|---|---|---|---|---|---|
| | | | | | | | GenBank |
| | None | None | Duac | Duac | Duac | | Accession or |
| probeset | NS-day 1 | IN-day 1 | IN-week 2 | IN-week 5 | IN-week 8 | Description | Gene Symbol |
| 207356_at | 164 | 2912 | 1816 | 956 | 2273 | defensin, beta 4 | DEFB4 |
| 202917_s_at | 968 | 6393 | 4708 | 2940 | 4185 | S100 calcium binding protein A8 (calgranulin A) | S100A8 |
| 240901_at | 62 | 242 | 41 | 44 | 34 | Transcribed locus | AI741601 |
| 1566144_at | 377 | 1027 | 439 | 428 | 401 | SH3-domain GRB2-like 3 (FLJ41018) | AK098337 |
| 231747_at | 14 | 136 | 118 | 38 | 36 | cysteinyl leukotriene receptor 1 | CYSLTR1 |
| 218340_s_at | 34 | 178 | 150 | 62 | 94 | ubiquitin-like modifier activating enzyme 6 (previously | UBA6 |
| | | | | | | hypothetical protein FLJ10808) | |
| 231809_x_at | 156 | 916 | 270 | 181 | 141 | programmed cell death 7 | PDCD7 |
| 229630_s_at | 714 | 1543 | 866 | 920 | 763 | Wilms tumor 1 associated protein | WTAP |
| 218527_at | 99 | 130 | 173 | 25 | 32 | aprataxin | APTX |
| 209719_x_at | 21 | 1417 | 1597 | 582 | 1175 | serpin peptidase inhibitor, clade B (ovalbumin), | SERPINB3 |
| | | | | | | member 3 | |
| 219915_s_at | 85 | 144 | 175 | 33 | 51 | solute carrier family 16 (monocarboxylic acid | SLC16A10 |
| | | | | | | transporters), member 10 | |
| 202859_x_at | 112 | 956 | 481 | 729 | 278 | interleukin 8 | IL8 |
| 206595_at | 11860 | 4350 | 6518 | 4468 | 7425 | cystatin E/M | CST6 |
| 205185_at | 10270 | 5656 | 7687 | 5322 | 7485 | serine peptidase inhibitor, Kazal type 5 | SPINK5 |
| 201149_s_at | 353 | 84 | 153 | 25 | 302 | TIMP metallopeptidase inhibitor 3 (Sorsby fundus | TIMP3 |
| | | | | | | dystrophy, pseudoinflammatory) | |
| 231733_at | 3406 | 1312 | 1857 | 1230 | 2276 | caspase recruitment domain family, member 18 | CARD18 |
| | | | | | | (previously ICEBERG caspase-1 inhibitor) | |
| 202376_at | 413 | 378 | 261 | 171 | 340 | serpin peptidase inhibitor, clade A (alpha-1 | SERPINA3 |
| | | | | | | antiproteinase, antitrypsin), member 3 | |
| 232602_at | 1649 | 629 | 303 | 367 | 687 | WAP four-disulfide core domain 3 | WFDC3 |
| 202551_s_at | 310 | 94 | 229 | 73 | 195 | cysteine rich transmembrane BMP regulator 1 (chordin- | CRIM1 |
| | | | | | | like) | |
| 210715_s_at | 1053 | 732 | 782 | 565 | 792 | serine peptidase inhibitor, Kunitz type, 2 | SPINT2 |

TABLE 9
Average expression data of inflammatory acne lesions and normal skin controls at day 1 and treatment of Duac from 17 patients

| | Duac treatment | | | | | | |
|---|---|---|---|---|---|---|---|
| | | | | | | | GenBank |
| | None | None | Duac | Duac | Duac | | Accession or |
| probeset | NS-day 1 | IN-day 1 | IN-week 2 | IN-week 5 | IN-week 8 | Description | Gene Symbol |
| 202917_s_at | 968 | 6393 | 4708 | 2940 | 4185 | S100 calcium binding protein A8 (calgranulin A) | S100A8 |
| 240901_at | 62 | 242 | 41 | 44 | 34 | Transcribed locus | AI741601 |
| 1566144_at | 377 | 1027 | 439 | 428 | 401 | SH3-domain GRB2-like 3 (FLJ41018) | AK098337 |
| 231747_at | 14 | 136 | 118 | 38 | 36 | cysteinyl leukotriene receptor 1 | CYSLTR1 |
| 218340_s_at | 34 | 178 | 150 | 62 | 94 | ubiquitin-like modifier activating enzyme 6 (previously | UBA6 |
| | | | | | | hypothetical protein FLJ10808) | |
| 231809_x_at | 156 | 916 | 270 | 181 | 141 | programmed cell death 7 | PDCD7 |
| 229630_s_at | 714 | 1543 | 866 | 920 | 763 | Wilms tumor 1 associated protein | WTAP |
| 218527_at | 99 | 130 | 173 | 25 | 32 | aprataxin | APTX |
| 209719_x_at | 21 | 1417 | 1597 | 582 | 1175 | serpin peptidase inhibitor, clade B (ovalbumin), | SERPINB3 |
| | | | | | | member 3 | |
| 219915_s_at | 85 | 144 | 175 | 33 | 51 | solute carrier family 16 (monocarboxylic acid | SLC16A10 |
| | | | | | | transporters), member 10 | |
| 206595_at | 11860 | 4350 | 6518 | 4468 | 7425 | cystatin E/M | CST6 |
| 205185_at | 10270 | 5656 | 7687 | 5322 | 7485 | serine peptidase inhibitor, Kazal type 5 | SPINK5 |
| 201149_s_at | 353 | 84 | 153 | 25 | 302 | TIMP metallopeptidase inhibitor 3 (Sorsby fundus | TIMP3 |
| | | | | | | dystrophy, pseudoinflammatory) | |
| 231733_at | 3406 | 1312 | 1857 | 1230 | 2276 | caspase recruitment domain family, member 18 | CARD18 |
| | | | | | | (previously ICEBERG caspase-1 inhibitor) | |
| 202376_at | 413 | 378 | 261 | 171 | 340 | serpin peptidase inhibitor, clade A (alpha-1 | SERPINA3 |
| | | | | | | antiproteinase, antitrypsin), member 3 | |
| 232602_at | 1649 | 629 | 303 | 367 | 687 | WAP four-disulfide core domain 3 | WFDC3 |
| 202551_s_at | 310 | 94 | 229 | 73 | 195 | cysteine rich transmembrane BMP regulator 1 (chordin- | CRIM1 |
| | | | | | | like) | |
| 210715_s_at | 1053 | 732 | 782 | 565 | 792 | serine peptidase inhibitor, Kunitz type, 2 | SPINT2 |

The examples and embodiments described herein are for illustrative purposes only and various modifications or changes suggested to persons skilled in the art are to be included within the spirit and purview of this application and scope of the appended claims.

What is claimed is:

1. A method for determining a response of a subject to treatment of acne, the method comprising:
a) providing a first sample from the subject comprising a gene product expressed by one or more genes listed in Tables 3, 6, 7, 8, or 9;
b) determining a first expression level of the gene product present in the first sample; and
c) characterizing the subject as responsive or not responsive to the treatment based on the first expression level of the gene product present in the first sample.

2. The method of claim 1, provided that the first sample is obtained from the subject before treatment.

3. The method of claim 2, provided that the subject is characterized as likely to respond to the treatment or not likely to respond to the treatment based on the expression level of the gene product in the first sample.

4. The method of claim 2, further comprising: providing a second sample from the subject obtained during and/or after treatment, provided that the second sample comprises the gene product expressed by the one or more genes listed in Tables 3, 6, 7, 8, or 9; determining a second expression level of the gene product present in the second sample; and comparing the first expression level of the gene product with the second expression level of the gene product, thereby characterizing the subject as responsive or not responsive to the treatment.

5. The method of claim 1, provided that the first sample is an epidermal sample obtained by applying to an acne lesion of the subject an adhesive tape in a manner sufficient to adhere a sample of the acne lesion to the adhesive tape, and removing the adhesive tape from the acne lesion in a manner sufficient to retain the adhered sample of the acne lesion to the adhesive tape.

6. The method of claim 1, provided that the gene product is one that has increased expression in an inflammatory acne lesion compared to normal skin.

7. The method of claim 1, provided that the gene product is one that has decreased expression in an inflammatory acne lesion compared to normal skin.

8. The method of claim 1, provided that characterizing the subject comprises comparing the first expression level of the gene product to an expression level of the gene product in a control sample.

9. The method of claim 8, provided that the control sample is a sample obtained from an inflammatory acne lesion.

10. The method of claim 8, provided that the control sample is a sample obtained from a normal skin sample.

11. The method of claim 8, provided that the first sample is obtained from the subject during or after treatment and the control sample is a sample obtained from the subject prior to treatment.

12. The method of claim 11, provided that the control sample is from an acne lesion.

13. The method of claim 1, provided that the gene product comprises a nucleic acid molecule.

14. The method of claim 13, provided that the nucleic acid molecule is a RNA molecule; and provided that determining the first expression level of the gene product comprises:
a) generation of a cDNA molecule from the RNA molecule; and
b) amplification the cDNA molecule by real-time quantitative PCR.

15. The method of claim 13, provided that determining the first expression level of the gene product comprises:
a) application of one or more probes to the first sample; provided that the one or more probes comprise nucleic acid sequences specific and complementary to one or more gene products expressed from one or more genes listed in Tables 3, 6, 7, 8, or 9; and
b) hybridization of the one or more probes to the gene expression product in the first sample.

16. The method of claim 1, provided that the treatment comprises administering to the subject an antiseptic, antibiotic, retinoid, hormone, anti-inflammatory agent, aldosterone receptor antagonist, comedo extraction, surgery, dermabrasion, phototherapy, or a combination thereof.

17. A method for characterizing a tissue, the method comprising:
a) providing an epidermal sample from a subject suspected of developing an acne lesion, the epidermal sample comprising a gene product expressed by one or more genes listed in Tables 3, 6, 7, 8, or 9;
b) determining an expression level of the gene product present in the epidermal sample; and
c) characterizing the subject as having or not having sensitivity to developing an acne lesion based on the expression level of the gene product in the epidermal sample.

18. The method of claim 17, provided that the epidermal sample is obtained by applying to a region of skin suspected of developing an acne lesion an adhesive tape in a manner sufficient to adhere a sample of the skin region to the adhesive tape, and removing the adhesive tape from the skin region in a manner sufficient to retain the adhered skin region sample to the adhesive tape.

19. The method of claim 17, provided that the gene product is one that has increased expression in inflammatory acne lesions compared to normal skin.

20. The method of claim 17, provided that the gene product is one that has decreased expression in inflammatory acne lesions compared to normal skin.

21. The method of claim 17, provided that characterizing the subject comprises comparing the expression level of the gene product in the epidermal sample to an expression level of the gene product in a control.

22. The method of claim 21, provided that the control is a sample obtained from an inflammatory acne lesion.

23. The method of claim 21, provided that the control is a sample obtained from a normal skin sample.

24. The method of claim 17, provided that gene product comprises a nucleic acid molecule.

25. The method of claim 24, provided that the nucleic acid molecule is a RNA molecule; and provided that determining the expression level of the gene product comprises:
a) generation of a cDNA molecule from the RNA molecule; and
b) amplification the cDNA molecule by real-time quantitative PCR.

26. The method of claim 24, provided that determining the expression level of the gene product comprises:
a) application of one or more probes to the epidermal sample; provided that the one or more probes comprise nucleic acid sequences specific and complementary to one or more gene products expressed from one or more genes listed in Tables 3, 6, 7, 8, or 9; and
b) hybridization of the one or more probes to the gene expression product in the epidermal sample.

27. The method of claim 17, provided that the subject is characterized as having sensitivity to developing an acne lesion, the method further comprising administering to the subject a treatment.

28. The method of claim 27, provided that the treatment comprises an antiseptic, antibiotic, retinoid, hormone, anti-inflammatory agent, aldosterone receptor antagonist, comedo extraction, surgery, dermabrasion, phototherapy, or a combination thereof.

29. A kit comprising:
a) a skin sample collection device comprising an adhesive tape; and
b) one or more primers, one or more probes, or a combination thereof, that selectively bind to a gene product expressed from one or more genes listed in any of Tables 3, 6, 7, 8, or 9.

30. The kit of claim 29, further comprising a cosmetic formulation for increasing or decreasing the expression of the one or more genes listed in any of Tables 3, 6, 7, 8, or 9.
